(19) United States
(12) Patent Application Publication
(10) Pub. No.: US 2024/0167096 A1
(43) Pub. Date: May 23, 2024

(54) PLASMA CELL-FREE RNA AND METHODS OF USE THEREOF AS NON-INVASIVE BIOMARKERS FOR ALZHEIMER'S DISEASE

(71) Applicants: Laura Ibanez, St. Louis, MO (US); Carlos Cruchaga, St. Louis, MO (US); Alejandro Cisterna Garcia, St. Louis, MO (US)

(72) Inventors: Laura Ibanez, St. Louis, MO (US); Carlos Cruchaga, St. Louis, MO (US); Alejandro Cisterna Garcia, St. Louis, MO (US)

(73) Assignee: Washington University, St. Louis, MO (US)

(21) Appl. No.: 18/516,523

(22) Filed: Nov. 21, 2023

Related U.S. Application Data
(60) Provisional application No. 63/384,473, filed on Nov. 21, 2022.

(51) Int. Cl. C12Q 1/6883

(52) U.S. Cl. CPC C12Q 1/6883; C12Q 2600/106; C12Q 2600/112; C12Q 2600/158

(57) ABSTRACT

The present disclosure provides methods for determining a type of neurodegenerative disease in a subject, selecting a treatment for a subject having a neurodegenerative disease, and detecting Alzheimer's Disease in a subject independent of amyloid beta. Methods includes providing a biological sample obtained from the subject, measuring a level of at least one gene-associated cfRNA in the biological sample, and determining a type of neurodegenerative disease, selecting a treatment for a subject having a neurodegenerative disease, or detecting Alzheimer's Disease based on the level of the at least one gene-associated cfRNA. In minimally-invasive embodiments, the biological sample is blood and the level of gene-associated cfRNA is a level of gene-associated plasma cfRNA. Some embodiments further include determining whether Alzheimer's Disease in a subject is preclinical, early symptomatic, or clinical Alzheimer's Disease.

### CROSS-REFERENCE TO RELATED APPLICATIONS

This application claims priority from U.S. Provisional Application Ser. No. 63/384,473 filed on 21 Nov. 2022, which is incorporated herein by reference in its entirety.

### STATEMENT REGARDING FEDERALLY SPONSORED RESEARCH OR DEVELOPMENT

This invention was made with government support under AG005681, AG003991, AG026276, and AG062723 awarded by the National Institutes of Health. The government has certain rights in the invention.

### MATERIAL INCORPORATED-BY-REFERENCE

Not applicable.

### FIELD

The present disclosure generally relates to blood-based biomarkers for detection and prediction of Alzheimer's Disease.

### BACKGROUND

Alzheimer's disease (AD) is a complex neurodegenerative disorder clinically characterized by gradual and progressive memory loss and, pathologically by the presence of senile plaques (amyloid-beta deposits) and neurofibrillary tangles (tau deposits) in the brain. Economically, it has been estimated that AD and other dementias cost approximately $355 billion in 2021, a cost that has been estimated to increase to $1.1 trillion in 2050. The availability of an early and accurate diagnostic tool for AD might save $7.9 trillion in medical and care costs. Currently, many efforts are being directed to find cost-effective and non-invasive biomarkers for AD that can be used to identify individuals at the presymptomatic stage, and patients at early symptomatic stages of the disease (preclinical AD individuals or mild cognitive impairment-MCI.

Imaging and cerebrospinal fluid (CSF) biomarkers are commonly used for Alzheimer's Disease (AD) diagnosis. The most used and accurate CSF biomarker is the amyloid β42/amyloid 340 (Aβ42/Aβ40) ratio which can correctly diagnose 82.8% of the screened AD patients. Additionally, the Aβ42/Aβ40 measurements in CSF are specific and allow differentiation of AD from dementia with Lewy bodies (DLB), Parkinson's disease (PD), and vascular dementia (VaD). However, the standardization of the measurements to use in the clinical practice has been challenging, mainly due to inter-laboratory differences in sample handling and analytical methods. Along with Aβ measurements, CSF levels of phosphorylated tau (p-tau), and total tau (t-tau) in CSF or brain are also used to aid AD diagnosis. T-tau is elevated in other neurodegenerative diseases such as DLB, frontotemporal degeneration (FTD), VaD, and Creutzfeldt-Jacob disease (CJD). In contrast, certain CSF p-tau species such as p-tau181 and p-tau231 are more specific to AD and show strong correlations with the tau PET. To improve the AD diagnosis, the Amyloid (A) Tau (T) Neurodegeneration (N) framework proposed a biological classification of AD into eight profiles according to positivity/negativity of three biomarkers, Aβ (A), p-tau (T), and t-tau (N). It is accepted that turning positive for Aβ means the beginning of the AD continuum. The increase in the number of positive biomarkers for the ATN criteria correlates with more advanced pathology, and it is associated with increased risk of dementia and cognitive decline. One of the main challenges for the ATN criteria is the definition of cut-off values for the biomarkers, especially for the triage of presymptomatic AD individual.

### SUMMARY OF THE DISCLOSURE

Among the various aspects of the present disclosure is the provision of a plasma cell-free RNA (cfRNA) and its use as a non-invasive biomarker for Alzheimer's Disease prediction and detection. As shown herein, disease-specific transcriptomic blood-based biomarkers identify Alzheimer's disease in the presymptomatic stages of the disease.

One aspect of the present disclosure provides for a method of determining a type of neurodegenerative disease in a subject. The method comprises: providing a biological sample obtained from the subject; measuring a level of at least one gene-associated cfRNA in the biological sample; and determining the type of neurodegenerative disease in the subject based on the level of the at least one gene-associated cfRNA.

In some embodiments, the type of neurodegenerative disease is selected from Alzheimer's Disease, Parkinson's disease, Lewy body dementia, and Frontotemporal dementia, and in further embodiments the Alzheimer's Disease is selected from is preclinical, early symptomatic, or clinical Alzheimer's Disease. In some embodiments, the subject has an APOE genotype risk factor for AD. In some embodiments, a level of amyloid beta in the subject is not measured, and the determining is not based on a level of amyloid beta in the subject. In some embodiments, the biological sample is blood and the level of gene-associated cfRNA is a level of gene-associated plasma cfRNA.

Another aspect of the present disclosure provides for a method of detecting Alzheimer's Disease in a subject. The method comprises: providing a biological sample obtained from the subject; measuring a level of at least one gene-associated cfRNA in the biological sample; and detecting Alzheimer's Disease in the subject based on the level of the at least one gene-associated cfRNA, wherein a level of amyloid beta in the subject is not measured and wherein the detecting is not based on a level of amyloid beta in the subject.

In some embodiments, the method further comprises determining whether the Alzheimer's Disease is preclinical, early symptomatic, or clinical Alzheimer's Disease. In some embodiments, the biological sample is blood and wherein the level of gene-associated cfRNA is a level of gene-associated plasma cfRNA. In further embodiments, the at least one gene-associated cfRNA comprises CYTH1, PRPF8, SND1, and SLC9A3R2; or the at least one gene-associated cfRNA comprises SYNPO; or the at least one gene-associated cfRNA comprises SYNPO, FP671120.3, JCAD, and PRPF8.

Another aspect of the present disclosure provides for a method of selecting a treatment for a subject having a neurodegenerative disease. The method comprises: providing a biological sample obtained from the subject; measuring a level of at least one gene-associated cfRNA in the biological sample; and selecting a treatment for the subject based on the level of the at least one gene-associated cfRNA.

In some embodiments, the neurodegenerative disease is Alzheimer's Disease and is selected from is preclinical, early symptomatic, or clinical Alzheimer's Disease. In some embodiments, the biological sample is blood and wherein the level of gene-associated cfRNA is a level of gene-associated plasma cfRNA. In further embodiments, the at least one gene-associated cfRNA comprises CYTH1, PRPF8, SND1, and SLC9A3R2; or the at least one gene-associated cfRNA comprises SYNPO; or the at least one gene-associated cfRNA comprises SYNPO, FP671120.3, JCAD, and PRPF8.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1(A-D) is an exemplary embodiment of presymptomatic Alzheimer Disease prediction results in accordance with the present disclosure. FIG. 1A shows study design summary showing the sample selection approach (retrospective clinical record review), the groups and subgroups included in the discovery and replication, along with other neurodegenerative diseases. FIG. 1B shows summary demographics for the discovery (training) and replication (testing) datasets. FIG. 1C is a whisker plot showing the performance of the prediction of presymptomatic AD in the replication/testing dataset for the three predictive models (40, 90, and 220 transcripts) with and without APOE genotype. APOE genotype predictive power is depicted at the bottom for reference FIG. 1D is an intersection matrix showing the shared and private transcripts among the three different models for presymptomatic AD.

FIG. 2 is an exemplary embodiment of differential expression results for the comparison of cfRNA in presymptomatic Alzheimer's Disease and controls in accordance with the present disclosure. The volcano plot shows the results from the discovery differential expression. Highlighted in blue are the ones that replicate in Toden et al; in green the ones that replicated in brain, and in black those that are DE in plasma and brain.

FIG. 3 is an exemplary embodiment of a predictive model design approach to minimize the batch effect between discovery and replication datasets, and machine learning application, in accordance with the present disclosure.

FIG. 4 is an exemplary embodiment of Kullback-Leibler divergence (KL) value threshold effect on the accuracy of the cross-validation experiments by the number of transcripts in accordance with the present disclosure. We explored predictive models built using different KL thresholds (from KL=0.06 to KL=0.36 by 0.02 increments) and different number of transcripts based on their rank (40, 65, 90, 120, 150, 180, 220, 250). Each line indicates a KL value, x-axis indicates the number of transcripts in the model and y-axis the accuracy obtained by those transcripts in the cross-validation experiments.

FIG. 5(A-C) is an exemplary embodiment of feature importance for each predictive models in accordance with the present disclosure. X-axis indicates beta values from the L2 regression. Y-axis indicates the predictor (transcript). Bars to the left of zero indicate when beta values are negative (i.e. the predictor is less expressed in presymptomatic AD) and bars to the right of zero indicate beta positive values (i.e. the predictor has higher expression in presymptomatic AD). FIG. 5A shows feature importance for predictive model with 40 transcripts. FIG. 5B shows feature importance for predictive model with 90 transcripts. FIG. 5C shows feature importance for predictive model with 220 transcripts.

FIG. 6(A-D) is an exemplary embodiment of sensitivity analyses for the predictive models in the AD continuum and in the context of the ATN framework in accordance with the present disclosure. FIG. 6A shows summary demographics for the early symptomatic and the symptomatic individuals. FIG. 6B is a whisker plot showing the performance of the prediction of early symptomatic AD and symptomatic AD for the three predictive models (40, 90, and 220 transcripts) with and without APOE genotype. FIG. 6C is a whisker plot showing the performance of the prediction of A+T+vs A−T− for the three predictive models (40, 90, and 220 transcripts) with and without APOE genotype. FIG. 6D is a whisker plot showing the performance of the prediction of CSF amyloid beta positivity for the three predictive models (40, 90, and 220 transcripts) with and without APOE genotype.

FIG. 7 is an exemplary embodiment of correlation matrix among levels of CSF AD biomarkers (tau, p-tau and Aβ42) and the predictive models in accordance with the present disclosure. The pies indicate the Spearman's rank correlation coefficient between the features, blue for negative, orange for positive. Value of correlation is on top of each pie. The right side of the matrix indicates the p-value corresponding to each correlation.

FIG. 8(A-C) is an exemplary embodiment of sensitivity analyses for the predictive models in other neurodegenerative diseases in accordance with the present disclosure. FIG. 8A shows summary demographics for the individuals from other neurodegenerative diseases. FIG. 8B is a whisker plot showing the performance of the prediction of other neurodegenerative diseases compared to controls for the three predictive models (40, 90, and 220 transcripts) with and without APOE genotype. FIG. 8C is a whisker plot showing the performance of the prediction of other neurodegenerative diseases compared to AD for the three predictive models (40, 90, and 220 transcripts) with and without APOE genotype.

FIG. 9 is an exemplary embodiment of Log₂Flod change values in accordance with the present disclosure. Log₂Flod change values are shown for the comparison between each neurodegenerative disease and the controls for all transcripts included in the three predictive models in each neurodegenerative disease. Sorted in decreasing order by their log₂Fold Change in the comparison of AD vs Control.

### DETAILED DESCRIPTION

The present disclosure is based, at least in part, on the discovery that plasma cell-free RNA (cfRNA) signatures are suitable as non-invasive biomarkers and as a diagnostic tool for Alzheimer's Disease (AD) detection and prediction, at least at preclinical stages.

There is a need of affordable, scalable, and specific blood-based biomarkers for Alzheimer's disease that can be applied to a population level. We have developed and validated disease-specific cell-free transcriptomic blood-based biomarkers composed by a scalable number of transcripts that capture AD pathobiology even in the presymptomatic stages of the disease. Accuracies are in the range of the current CSF and plasma biomarkers, and specificities are high against other neurodegenerative diseases.

As shown herein, plasma cfRNA signatures can be used to detect and predict Alzheimer's Disease via less invasive (blood-based as opposed to cerebrospinal fluid based), less expensive, and accurate biomarker testing, as shown at least in Example 1.

The present disclosure relates, in general, to methods for detecting and predicting Alzheimer's Disease occurrence. More specifically, the present disclosure provides methods to quantify plasma cfRNA to guide treatment decisions, evaluate the clinical efficacy of certain therapeutic interventions, and select subjects for clinical trials.

The Amyloid Tau Neurodegeneration (A/T/N) classification system is currently the best method to diagnose Alzheimer's Disease (AD), although the final definitive diagnostic tool is post-mortem pathology. A/T/N biomarkers are used to clinically diagnose AD, those biomarkers obtain an accuracy of 70-85% for clinical stages of AD. However, to date A/T/N does not enable final prediction of preclinical AD individuals and specificity analyses with other neurodegenerative diseases.

Disclosed herein is a new process that enables the early detection of AD patients at preclinical stages. It is based on human RNA extraction from plasma samples, followed by whole transcriptome sequencing, and the development of a prediction model using machine learning techniques. Performing quality control and normalization processes using Deseq2 and Z-scores enables computation of the Kullback-Leibler divergence (KL) between cohorts. After that, different KL thresholds were used to select genes as predictors and then Ridge regression to find the best predictive model. This model includes 220 genes and it can (statistically significantly, P-Value=1.377e-07) classify between non-AD individuals (control participants) and preclinical AD patients from a testing cohort with an accuracy of 95.24% 95Cl [84-99%], a sensitivity of 95.45%, and a specificity of 95.00%. Overall, applying the model to the total cohort (67 preclinical AD and 48 controls) correctly classified 61 preclinical AD and 44 controls. The predictive AD risk of the model consistently correlates with Aβ42 levels, one of the biomarkers used in the A/T/N classification system. Moreover, the specificity of the model was tested for other neurodegenerative diseases, and specificity was observed for AD in comparison to Parkinson's disease, dementia with Lewy bodies, and frontotemporal dementia. In addition, a signature was also detected of around 1500 genes associated with preclinical AD.

In summary, the methods disclosed herein can be built into a cost-effective and non-invasive definitive diagnostic tool for AD. In some embodiments, the detection and prediction methods described herein are useful for early triage on AD clinical trials and drug monitoring since the model does not measure or require Amyloid beta.

Chemical Agent:

Examples of chemical agents, therapeutic formations, and therapeutic compositions in accordance with the present disclosure can include at least one compound or a pharmaceutically acceptable salt, solvate, polymorph, tautomer, prodrug, analog, or stereoisomer thereof or optionally substituted analog thereof.

The formulas, analogs, and R groups can be optionally substituted or functionalized with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxyl; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “carbonyl”, as used herein, unless otherwise indicated, can include a functional group consisting of a carbon atom double-bonded to an oxygen atom (C═O). The “carbonyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O— isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O— propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O— cyclooctyl, —O-cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S, and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “heterocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example, water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “UM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.,”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or another counterion. The counterion may be any organic or inorganic moiety that stabilizes the charge on the parent compound. Furthermore, a pharmaceutically acceptable salt may have more than one charged atom in its structure. In instances where multiple charged atoms are part of the pharmaceutically acceptable salt, the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| Base | Name | Bases Represented | Complementary Base |
|---|---|---|---|
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | puRine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes. An operon is a cluster of genes that are transcribed together to give a single messenger RNA (mRNA) molecule, which therefore encodes multiple proteins.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence (or polynucleotide sequence) must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2A) can be used in a construct to prevent covalently linking translated amino acid sequences.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using self-replicating primers, paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted or deleted from a DNA or RNA sequence of an organism's genome. Point mutations have a variety of effects on the downstream protein product-consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild-type codon in that position. In some silent mutations the codon codes for a different amino acid that happens to have the same properties as the amino acid produced by the wild-type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| | |
|---|---|
| Side Chain Characteristic | Amino Acid |

Non-polar (hydrophobic)

| | |
|---|---|
| A. Aliphatic: | A L I V P |
| B. Aromatic: | F W |
| C. Sulfur-containing: | M |
| D. Borderline: | G |

Uncharged-polar

| | |
|---|---|
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Genome Editing

As described herein, various signals can be modulated (e.g., reduced, eliminated, or enhanced) using genome editing.

As described herein, activity, signals, expression, or function can be modulated (e.g., reduced, eliminated, or enhanced) using genome editing (e.g., upregulate, downregulate, overexpress, underexpress, express (e.g., transgenic expression), knock in, knock out, knockdown).

Processes for genome editing are well known; see e.g., Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage by genome editing can result in protection from various diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications to target cells by the removal or addition of signals (e.g., activate (e.g., CRISPRa), upregulate, overexpress, downregulate).

For example, the methods as described herein can comprise a method for altering a target polynucleotide sequence in a cell comprising contacting the polynucleotide sequence with a clustered regularly interspaced short palindromic repeats-associated (Cas) protein.

Gene Therapy and Genome Editing

Gene therapies can include inserting a functional gene with a viral vector.

There has recently been an improved landscape for gene therapies. For example, in the first quarter of 2019, there were 372 ongoing gene therapy clinical trials (Alliance for Regenerative Medicine, May 9, 2019).

Any vector known in the art can be used. For example, the vector can be a viral vector selected from retrovirus, lentivirus, herpes, adenovirus, adeno-associated virus (AAV), rabies, Ebola, lentivirus, or hybrids thereof.

Gene therapy strategies.

| | Strategy |
|---|---|
| Viral Vectors | |
| Retroviruses | Retroviruses are RNA viruses transcribing their |
| | single-stranded genome into a double-stranded DNA |
| | copy, which can integrate into host chromosome |
| Adenoviruses | Ad can transfect a variety of quiescent and |
| (Ad) | proliferating cell types from various species and |
| | can mediate robust gene expression |
| Adeno-associated | Recombinant AAV vectors contain no viral DNA and |
| Viruses (AAV) | can carry ~4.7 kb of foreign transgenic material. |
| | They are replication defective and can replicate |
| | only while coinfecting with a helper virus |
| Non-viral vectors | |
| plasmid DNA | pDNA has many desired characteristics as a gene |
| (pDNA) | therapy vector; there are no limits on the size or |
| | genetic constitution of DNA, it is relatively |
| | inexpensive to supply, and unlike viruses, |
| | antibodies are not generated against DNA in |
| | normal individuals |
| RNAi | RNAi is a powerful tool for gene specific silencing |
| | that could be useful as an enzyme reduction therapy |
| | or means to promote read-through of a premature stop |
| | codon |

Gene therapy can allow for the constant delivery of the enzyme directly to target organs and eliminates the need for weekly infusions. Also, correction of a few cells could lead to the enzyme being secreted into the circulation and taken up by their neighboring cells (cross-correction), resulting in widespread correction of the biochemical defects. As such, the number of cells that must be modified with a gene transfer vector is relatively low.

Genetic modification can be performed either ex vivo or in vivo. The ex vivo strategy is based on the modification of cells in culture and transplantation of the modified cell into a patient. Cells that are most commonly considered therapeutic targets for monogenic diseases are stem cells. Advances in the collection and isolation of these cells from a variety of sources have promoted autologous gene therapy as a viable option.

The use of endonucleases for targeted genome editing can solve the limitations presented by the usual gene therapy protocols. These enzymes are custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating, preventing, or reversing Alzheimer's Disease, preclinical Alzheimer's Disease, or other neurodegenerative disease in a subject in need thereof by administration of a therapeutically effective amount of a therapeutic formulation or composition based on detection of cfRNA in the subject.

Methods described herein are generally performed on a subject in need thereof. A subject in need of the therapeutic methods described herein can be a subject having, diagnosed with, suspected of having, or at risk for developing Alzheimer's Disease, preclinical Alzheimer's Disease, or other neurodegenerative disease or condition. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Neurodegenerative Disease

The compositions and methods as described herein can be used to treat a neurodegenerative disease, disorder, or condition.

For example, a neurodegenerative disease, disorder or condition can be a hereditary motor and sensory neuropathy (HMSN) (e.g., Charcot Marie Tooth (CMT) disease), CMT1 (a dominantly inherited, hypertrophic, predominantly demyelinating form), CMT2 (a dominantly inherited predominantly axonal form), Dejerine-Sottas (severe form with onset in infancy), CMTX (inherited in an X-linked manner), CMT4 (includes the various demyelinating autosomal recessive forms of Charcot-Marie-Tooth disease), hereditary sensory and autonomic neuropathy type IE, hereditary sensory and autonomic neuropathy type II, hereditary sensory and autonomic neuropathy type V, HMSN types 1A and 1B (e.g., dominantly inherited hypertrophic demyelinating neuropathies), HMSN type 2 (e.g., dominantly inherited neuronal neuropathies), HMSN type 3 (e.g., hypertrophic neuropathy of infancy [Dejerine-Sottas]), HMSN type 4 (e.g., hypertrophic neuropathy [Refsum] associated with phytanic acid excess), HMSN type 5 (associated with spastic paraplegia), or HMSN type 6 (e.g., with optic atrophy).

As another example, a neurodegenerative disease, disorder or condition can be Alzheimer's disease, amyotrophic lateral sclerosis (ALS), Alexander disease, Alpers' disease, Alpers-Huttenlocher syndrome, alpha-methylacyl-CoA racemase deficiency, Andermann syndrome, Arts syndrome, ataxia neuropathy spectrum, ataxia (e.g., with oculomotor apraxia, autosomal dominant cerebellar ataxia, deafness, and narcolepsy), autosomal recessive spastic ataxia of Charlevoix-Saguenay, Batten disease, beta-propeller protein-associated neurodegeneration, Cerebro-Oculo-Facio-Skeletal Syndrome (COFS), Corticobasal Degeneration, CLN1 disease, CLN10 disease, CLN2 disease, CLN3 disease, CLN4 disease, CLN6 disease, CLN7 disease, CLN8 disease, cognitive dysfunction, congenital insensitivity to pain with anhidrosis, dementia, familial encephalopathy with neuroserpin inclusion bodies, familial British dementia, familial Danish dementia, fatty acid hydroxylase-associated neurodegeneration, Gerstmann-Straussler-Scheinker Disease, GM2-gangliosidosis (e.g., AB variant), HMSN type 7 (e.g., with retinitis pigmentosa), Huntington's disease, infantile neuroaxonal dystrophy, infantile-onset ascending hereditary spastic paralysis, Huntington's disease (HD), infantile-onset spinocerebellar ataxia, juvenile primary lateral sclerosis, Kennedy's disease, Kuru, Leigh's Disease, Marinesco-Sjögren syndrome, Mild Cognitive Impairment (MCI), mitochondrial membrane protein-associated neurodegeneration, Motor neuron disease, Monomelic Amyotrophy, Motor neuron diseases (MND), Multiple System Atrophy, Multiple System Atrophy with Orthostatic Hypotension (Shy-Drager Syndrome), multiple sclerosis, multiple system atrophy, neurodegeneration in Down's syndrome (NDS), neurodegeneration of aging, Neurodegeneration with brain iron accumulation, neuromyelitis optica, pantothenate kinase-associated neurodegeneration, Opsoclonus Myoclonus, prion disease, Progressive Multifocal Leukoencephalopathy, Parkinson's disease (PD), PD-related disorders, polycystic lipomembranous osteodysplasia with sclerosing leukoencephalopathy, prion disease, progressive external ophthalmoplegia, riboflavin transporter deficiency neuronopathy, Sandhoff disease, Spinal muscular atrophy (SMA), Spinocerebellar ataxia (SCA), Striatonigral degeneration, Transmissible Spongiform Encephalopathies (Prion Diseases), or Wallerian-like degeneration.

Generally, a safe and effective amount of a therapeutic formulation or composition is, for example, an amount that would cause the desired therapeutic effect in a subject while minimizing undesired side effects. In various embodiments, an effective amount of the therapeutic formulation or composition can substantially inhibit, slow the progress of, or limit the development of Alzheimer's Disease, preclinical Alzheimer's Disease, or other neurodegenerative disease.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a therapeutic formulation or composition can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat, prevent, or reverse Alzheimer's Disease, preclinical Alzheimer's Disease, or other neurodegenerative disease.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of a therapeutic formulation or composition can occur as a single event or over a time course of treatment. For example, a therapeutic formulation or composition can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for Alzheimer's Disease, preclinical Alzheimer's Disease, or other neurodegenerative conditions.

A therapeutic formulation or composition can be administered simultaneously or sequentially with another agent, such as an antibiotic, an anti-inflammatory, or another agent. For example, a therapeutic formulation or composition can be administered simultaneously with another agent, such as an antibiotic or an anti-inflammatory. Simultaneous administration can occur through administration of separate compositions, each containing one or more of a therapeutic formulation or composition, an antibiotic, an anti-inflammatory, or another agent. Simultaneous administration can occur through administration of one composition containing two or more of a therapeutic formulation or composition, an antibiotic, an anti-inflammatory, or another agent. A therapeutic formulation or composition can be administered sequentially with an antibiotic, an anti-inflammatory, or another agent. For example, a therapeutic formulation or composition can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the therapeutic formulation or composition may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a therapeutic formulation or composition may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

Cell Therapy

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogeneic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

Screening

Also provided are screening methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character×log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character×log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment.

In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Plasma Cell-Free RNA (cfRNA) Signatures Corresponding to Alzheimer's Disease Preclinical Stages

This example describes prediction of Alzheimer's Disease using plasma cfRNA signatures as non-invasive biomarkers in accordance with the present disclosure.

### Introduction

CSF and imaging biomarkers have proven helpful in the detection of AD; however, they are invasive and expensive. In consequence, the study of blood-based biomarkers has intensified in the last decade. These biomarkers are less invasive and may provide comparable accuracy to CSF and imaging measures. For example, plasma t-tau was found to be higher in the advanced stages of AD, but as with CSF measurements, it does not seem to be specific to AD. New evidence suggests that phosphorylated species of tau, especially p-tau 217 are specific to AD, with values increasing progressively from healthy individuals to MCI to AD. Further, Aβ has also been widely studied in plasma showing that the ratio of plasma Aβ42/Aβ40 highly correlates with brain amyloidosis, especially when measured with high-precision techniques and combined with APOE genotype, the main genetic risk factor for AD. However, cost-effectiveness and scalability of these measurements are not optimal.

Most blood-based biomarker studies measure protein levels; however, nucleic acids can also be used as biomarkers. The cell-free DNA (cfDNA) diagnostic test, which allows the detection of genetic disorders and chromosome abnormalities during pregnancy, revolutionized prenatal screening by avoiding procedure-related miscarriage risks. Plasma also contains ribonucleic acid in its free form (cell-free RNA-cfRNA) that has the potential to capture temporal processes since its source seems to be the result of normal cell death throughout the body. Several species of cfRNA have been intensively investigated as biomarkers for cancer, fetal development, and AD. While several studies proposed circulating microRNAs as AD biomarkers only one published study used plasma messenger cfRNA to capture transcriptomic alterations in advanced AD stages. In their comparisons between AD cases (n=122) and controls (n=116), they identified 2591 differential expressed (DE) transcripts. Then, they used the transcriptomic information to build classifiers to discriminate AD patients from healthy controls with an area under the curve (AUC) of 0.83. Even though promising, the models include most of the differentially expressed genes (1658), instead of a subset of the most informative genes, which would improve the scalability and facilitate the translation to a clinical setting. No study thus far has evaluated the use of cfRNA as a potential approach to develop clinically useful AD biomarkers for presymptomatic phases of the disease.

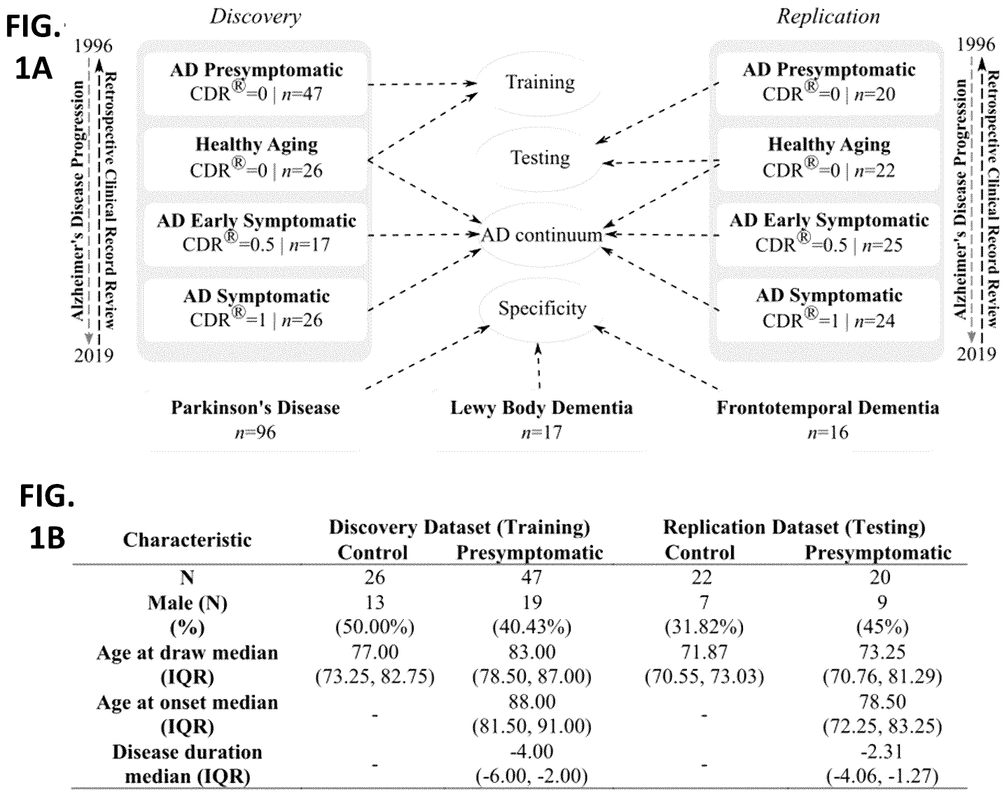

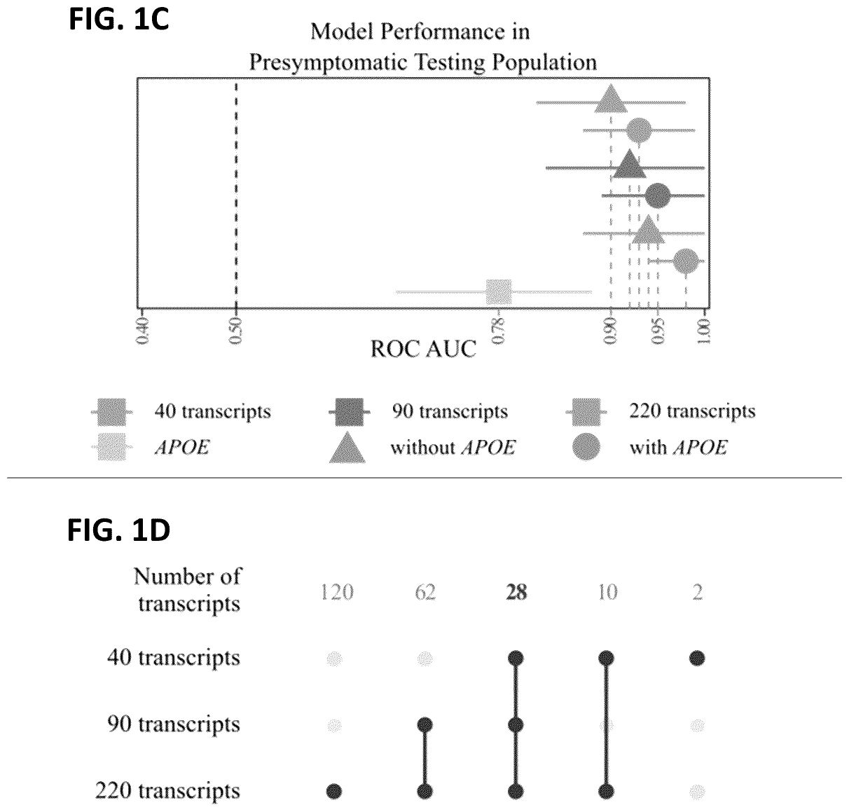

Here, as differentiated from previous studies, we leveraged plasma cfRNA of presymptomatic AD participants to capture the early changes caused by AD pathology and to build unbiased models that were able to balance a good performance with a scalable number of transcripts to facilitate potential clinical applications. We also evaluated the discriminative capabilities of the proposed AD presymptomatic models in the context of AD spectrum, PD, DLB, and Frontotemporal Dementia (FTD) to ensure that the models were selectively capturing changes associated with AD pathobiology (see FIG. 1A).

### Results

Concordance Between Dysregulated Transcripts in Plasma cfRNA and Brain of AD Participants

We analyzed plasma cfRNA from presymptomatic AD participants to capture the early changes caused by AD pathology and build classifiers containing the expression of a scalable number of genes. All presymptomatic AD participants were required to have a sample before onset of symptoms (time of draw), and evidence of Aβ deposition (CSF Aβ<500 ng/L or positive PET scan) and/or evidence of clinical worsening measured by Clinical Dementia Rating (CDR®) at the last clinical visit compared to the time of draw (18.0-6.5 years prior) (FIG. 1A). We generate two independent datasets, by conducting retrospective sample selection twice (one for discovery and another for replication, separated by four years) from the Knight-ADRC, a deeply phenotyped cohort with longitudinal data and samples available. Due to time difference, RNA extraction and library preparation protocols were different for the discovery compared to the replication datasets (see methods section). In summary, we extracted and sequenced RNA of non-fasted plasma samples from a total of 67 presymptomatic AD participants (n_{discovery}=47; n_{replication}=20) and 47 controls (n_{discovery}=26; n_{replication}=22) (FIGS. 1(A-B)).

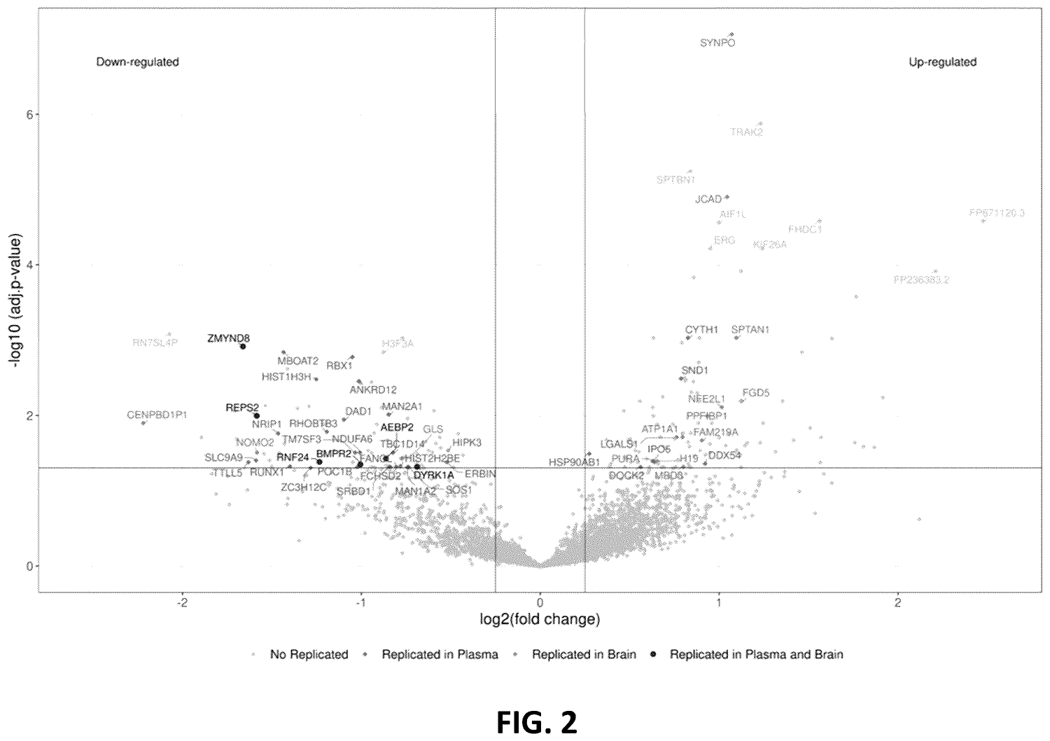

After stringent quality control (QC), we performed DE analyses comparing presymptomatic AD participants and controls using DESeq2 (46). We identified 190 DE transcripts while controlling for sex and age at draw (FIG. 2 and Table 1). We used previously identified DE transcripts found in plasma of advanced symptomatic AD to replicate 37 of our findings, which showed statistical significance for the overlap (p=0.01, Table 1). Most importantly, we wanted to know if the cfRNA was potentially capturing changes taking place in the brain. Using an in-house dataset we found that 23 out of 190 transcripts were DE in both the brain and plasma of AD participants (FIG. 2 and Table 1). The overlap was statistically significant (p=0.03) with a fold enrichment of 1.6. On top of that, the effect sizes of the 23 genes in the brain and the plasma were highly correlated (cor=0.83; p=7.55×10⁻⁰⁷). Overall, seven out of 190 the plasma DE transcripts were common between the 37 transcripts replicated in plasma and the 23 replicated in brain (MBOAT2, SLC9A9, RHOBTB3, RUNX1M, POC1B, SRBD1, and HIPK3). To further investigate if the transcripts identified in this study were expressed in the brain, we accessed the GTEx portal and found 176 out of the 190 genes to be expressed in brain cortex tissue (Table 1), adding evidence to the brain as a potential source of the DE cfRNA transcripts.

To assess the potential biological relevance of the 190 DE transcripts, we explored the Kyoto Encyclopedia of Genes and Genomes (KEGG) and found that the identified transcripts were enriched and significantly overlapped with the AD pathway (nine genes, p=8.92×10⁻³, Table 1). We also used the ToppFun tool from ToppGene Suite and found that the 190 transcripts are in concordance with transcripts up-regulated in the brains of patients with AD (p=1.40×10⁻⁴)⁶⁶. We also identified an enrichment in Gene Ontology (GO) terms cellular component neuronal synapse (p=6.69×10⁻³) and postsynapse (p=1.58×10 2). Finally, we performed a co-expression analysis using networks from the frontal cortex of AD cases from ROSMAP in the CoExpWeb (48). We found a statistically significant overlap between the 190 transcripts and two co-expression modules (thistle1 and darkgrey). The thistle1 module (p=2.00×10⁻⁴), was associated with oligodendrocytes in the cortex whereas the darkgrey module (p=0.03) was associated with vasculature development and endothelial-external cells. Taken together, plasma cfRNA is capturing metabolic processes happening in the brain and might be reflecting transcriptional changes related to AD pathology of presymptomatic AD participants.

cfRNA Recapitulates a Transcript Signature Corresponding to the Presymptomatic Stages of Alzheimer's Disease

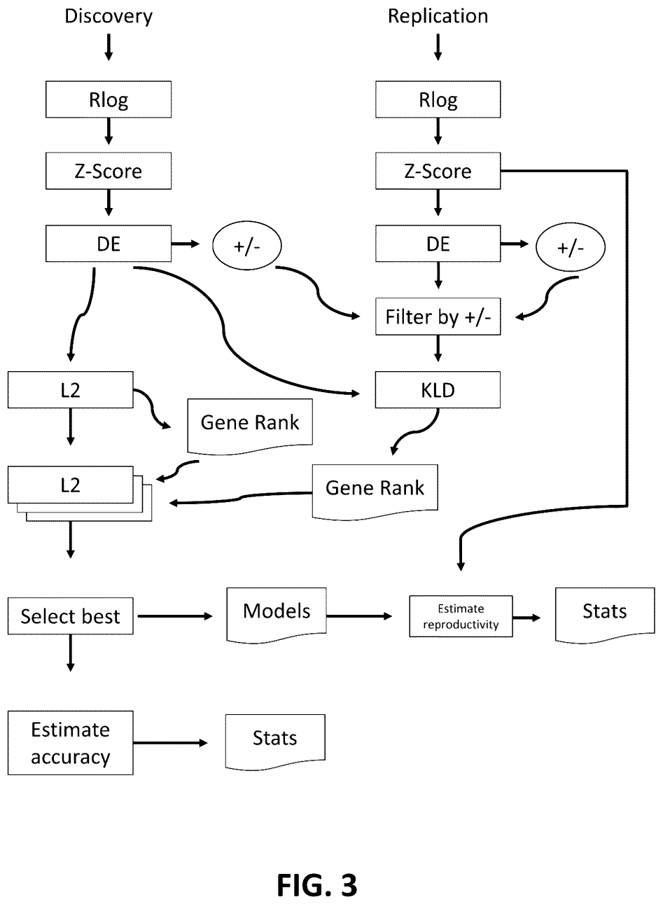

To leverage all the RNA data available, we have developed a new approach that allows the use of two independent RNA sequencing experiments as training (discovery) and testing (replication) for machine learning model development pipeline (FIG. 3). Briefly, we reduced the dimensionality of the two datasets by retaining transcripts showing the same direction of effect in the case-control comparison. Then, we calculated the distribution overlap of each transcript within the two datasets using the Kullback-Leibler divergence (KLD) and used their absolute values to rank the transcripts and generate eight subsets with diverse number of genes. Within each subset we used KLD thresholds (from 0.06 to 0.36 by increments of 0.02) and L2 regularization linear models (ridge regression) to predict presymptomatic AD in the training dataset (discovery). Then we evaluated the performance on the testing dataset.

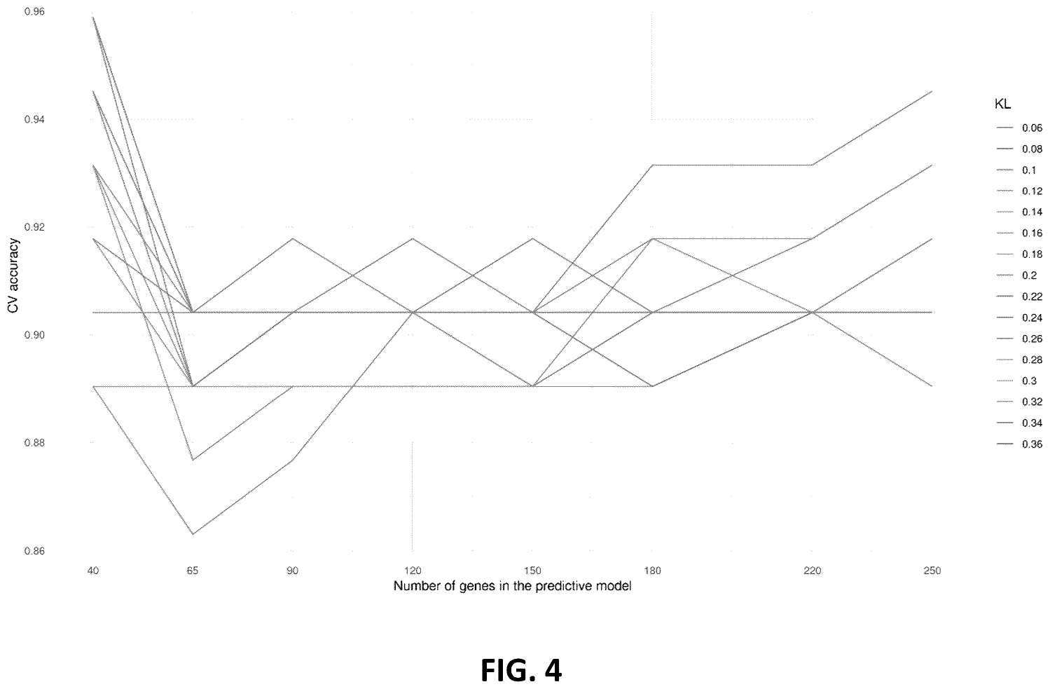

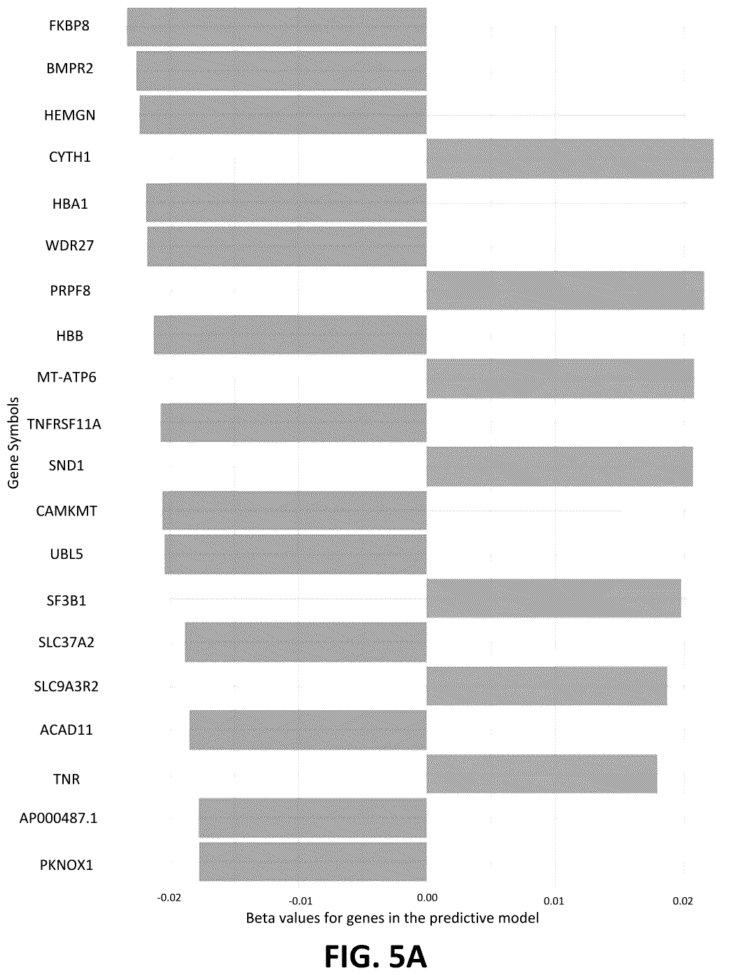

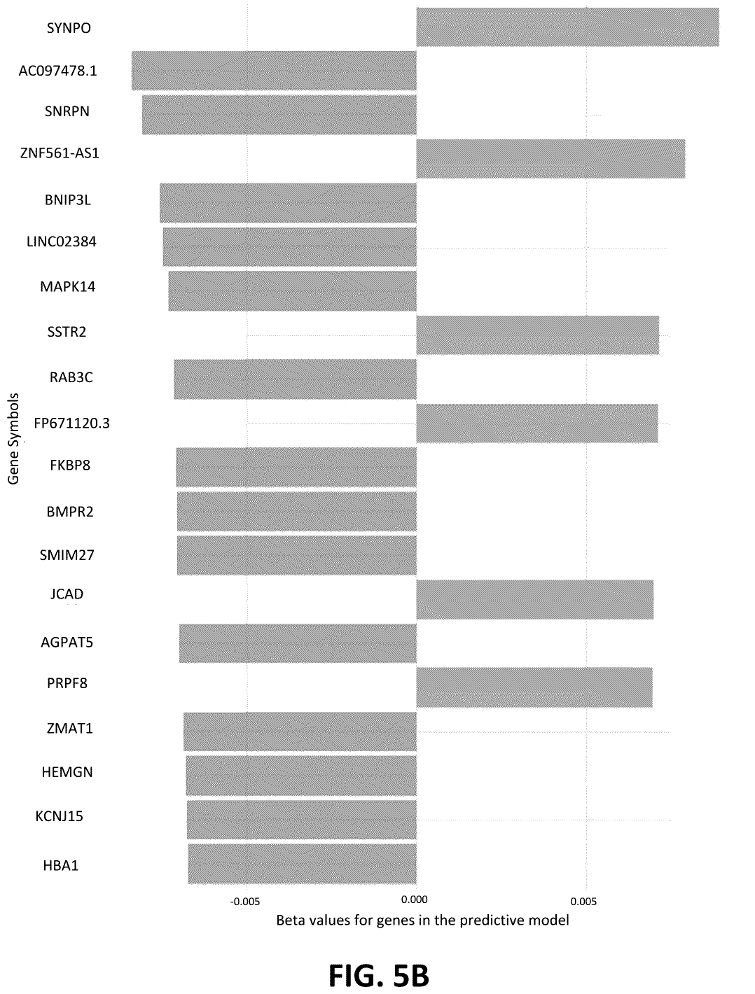

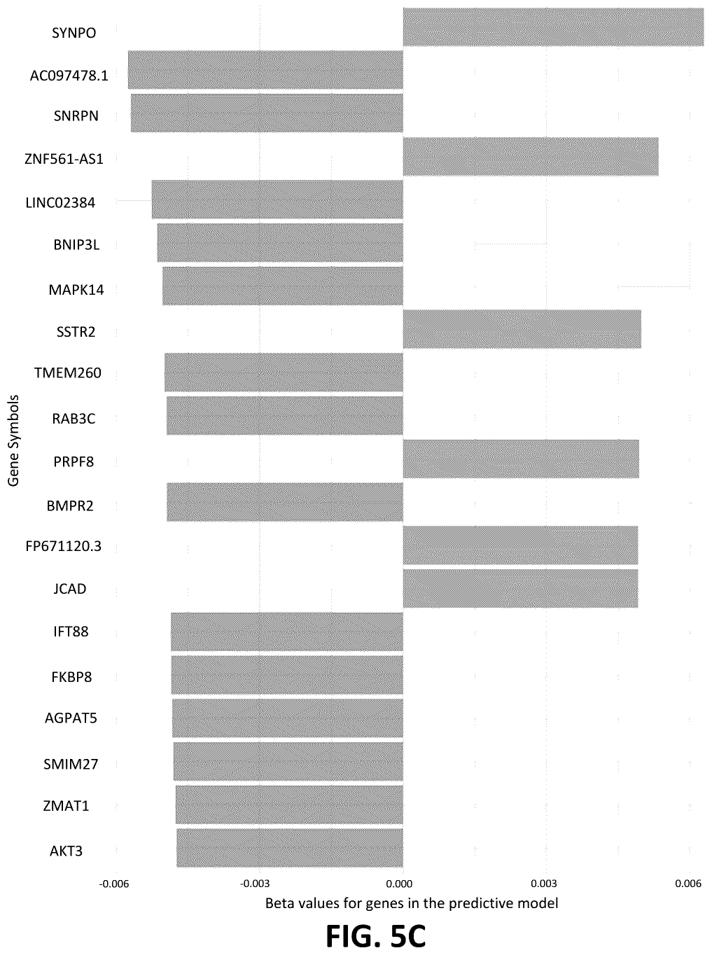

We generated a total of 272 models with different number of transcripts and then selected the best three based on the cross-validation experiment (FIG. 4). The best models contained 40, 90 and 220 transcripts with an area under the ROC curve (ROC) in the testing dataset of 0.90, 0.92, and 0.94 respectively (FIG. 1C and Table 2). We observed that the transcript overlap across models was significant (p<2.16×10⁻¹⁶, FIG. 1D), suggesting that the new standardization, described in detail in the methods section, and feature selection strategy implemented here tends to select good predictors in a consistent manner. In fact, the 28 common transcripts across the three models had an AUC of 0.92. After extracting the beta values (i.e., the importance of the predictors) from each gene in each of the predictive models (FIG. 5), we observed that the transcript corresponding to the gene SYNPO (the top hit from the DE analyses) was the most relevant feature for the models with 90 and 220 genes.

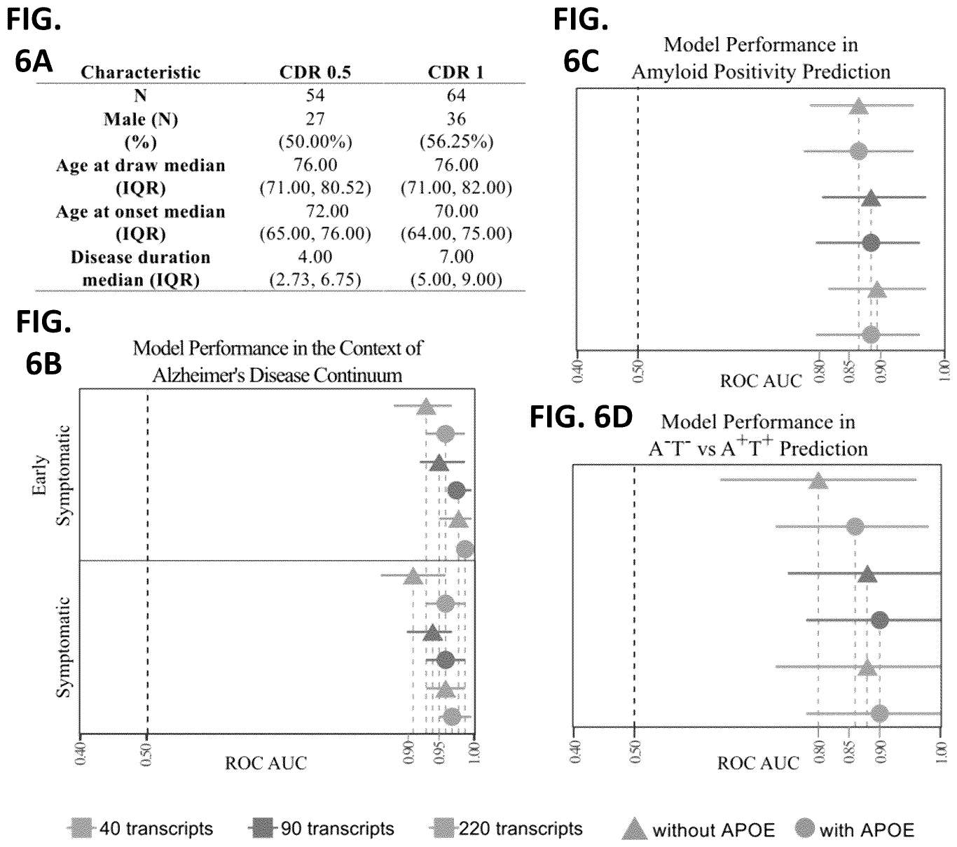

In previously published plasma biomarkers, the inclusion of APOE genotype in the model improved the performance. In our case, the addition of APOE genotype did not change the predictive power of the models, implying that we are already capturing the risk associated with APOE genotype (FIG. 1C and Table 2). Besides the ability to differentiate between presymptomatic AD participants and controls, we also tested the performance of the models within the AD continuum. We evaluated the accuracy of the three models (with and without APOE genotype) in early symptomatic (CDR®=0.5, n=42) and symptomatic AD (CDR®=1, n=50) (FIG. 6A and Table 3). In all cases, the AUC was greater than 0.90 (FIG. 6B and Table 4), suggesting that as AD progresses, its molecular signatures change but not drastically.

Predictive Models are Capturing Pathways Related to AD Early in the Disease Pathobiology

To understand the link between the transcripts included in the predictive models and their potential involvement in the pathobiology of AD we performed gene enrichment analyses for each of the models separately. Given the limited number of transcripts included in each of the three models, in order to add robustness to the enrichment analyses, we expanded each transcript set to include transcripts that show significant correlation (p<0.05 and r>0.95) with the transcripts of each predictive model (see methods section). Thus, the sets increased to 844, 1054, and 2436 for the predictive models including 40, 90, and 220 transcripts respectively, with several transcripts present in all of the sets. We identified 1201, 1111, and 494 overrepresented GO terms (Table 5). Relevant terms known to be associated with AD such as immune-related pathways and processes (GO term IDs: 0002218, 0002753, 0002757, and 0002764), or lysosome (GO term IDs: 0005765 and 0005766) were significant in all three analyses. We identified significant enrichment in terms related to the regulation of neuronal apoptosis and death (GO term IDs: 0043523, 0051402, 0070997, 1901214, 1901215, and 1901216) in all three analyses, supporting the capture of early neuropathological processes taking place in the brain by the predictive models. Similarly, KEGG enrichment analyses identified 78, 68, and 40 significantly overrepresented terms (Table 6) for each of the sets generated for each predictive model. Among others, neurodegenerative diseases including AD and PD were significantly enriched suggesting that we are in fact capturing processes related to the known biology of neurodegenerative disease early in the curse of the disease.

Predictive Models Trained with Presymptomatic AD Participants can Accurately Predict Amyloid Positivity

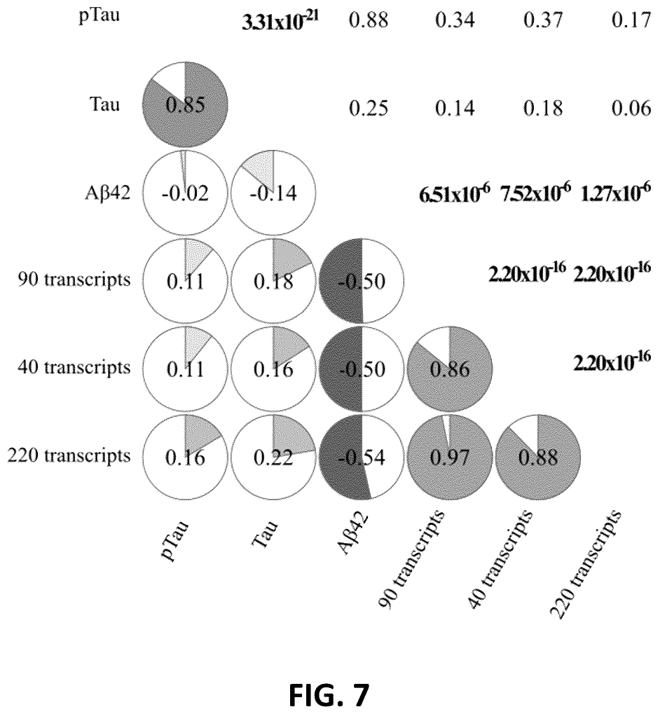

Current biomarkers evaluate the levels of Aβ42 in CSF or plasma to predict brain amyloidosis. We investigated if the estimated risk of AD calculated using the three models generated here (i.e. a number in the [0,1] interval) correlated with CSF Aβ42 levels. For those controls (n=43) and presymptomatic AD participants (n=28) with CSF measurements available at the time of blood draw, we tested if the AD risk calculated using the three models correlated with CSF Aβ42, tau, and p-tau (FIG. 7). We found significant associations with CSF Aβ42 levels, especially for the model containing 220 transcripts (r2=−0.54; p=1.27×10-6), but not with other CSF biomarkers or AD risk factors (FIG. 7). Associations with CSF Aβ42 had a negative direction, as expected. Finally, we classified these samples following the ATN criteria. Out of 72 samples, 49 were A⁻, and 23 A⁺, whereas 23 were T⁻ and 49 T⁺. Using the three transcriptomic models, we predicted A positivity status with AUCs of 0.89, 0.88 and 0.86 for the models with 220, 90 and 40 transcripts respectively (FIG. 6C). When including APOE to the models, we observed no changes of the AUCs, adding evidence to the fact that the transcriptomic model is capturing changes related to Alzheimer's disease and to its main pathology. We also tested the predictive performance for A⁺T⁺ compared to A⁻T⁻, even though the sample size was limited (n=13 participants in each group). The model with 40 transcripts was the one with the poorer performance with an AUC of 0.80, whereas the models with 90 and 220 transcripts had an AUC of 0.88 (FIG. 6D). In this case, including APOE improved the AUC in all cases, 0.86 for the model with 40 transcripts and 0.90 for the models including 90 and 220 transcripts.

Predictive Models Trained with Presymptomatic AD Participants can Also Predict AD in the Symptomatic Phases of the Disease

Besides the ability to differentiate between presymptomatic AD participants and controls; we also tested the performance of the models within the AD continuum (FIG. 1A). We evaluated the accuracy of the three models in early symptomatic (CDR®=0.5, n=42) and symptomatic (CDR®=1, n=50) AD compared to controls (n=48) (FIG. 6A). For early symptomatic AD participants, the AUC of the models composed by 40, 90, and 220 transcripts was 0.93, 0.95, and 0.98 respectively while for symptomatic AD the AUC were 0.91, 0.94, and 0.96 (FIG. 6B and Table 3). Differently from our results with the presymptomatic group, the addition of APOE genotype did improve the accuracy of the three models in the AD continuum (FIG. 6B and Table 3). However, the improvement was not accentuated, suggesting that we are still capturing some of the APOE genotype in the plasma transcriptome. Overall, the model with 40 transcripts showed less predictive power than models including additional transcripts. Although this could be a technical artifact of how linear models work (higher number of predictors tend to increase predictive power), our results may alternatively suggest that as AD progresses, the molecular signatures change, which has an impact on the accuracy of the predictive models. However, the addition of transcripts to the signature yield better AUC, suggesting that for some transcripts, the changes accentuate with disease progression, increasing the predictive ability of the models.

Predictive Models Trained with Presymptomatic AD Participants have Limited Ability to Predict Other Neurodegenerative Diseases

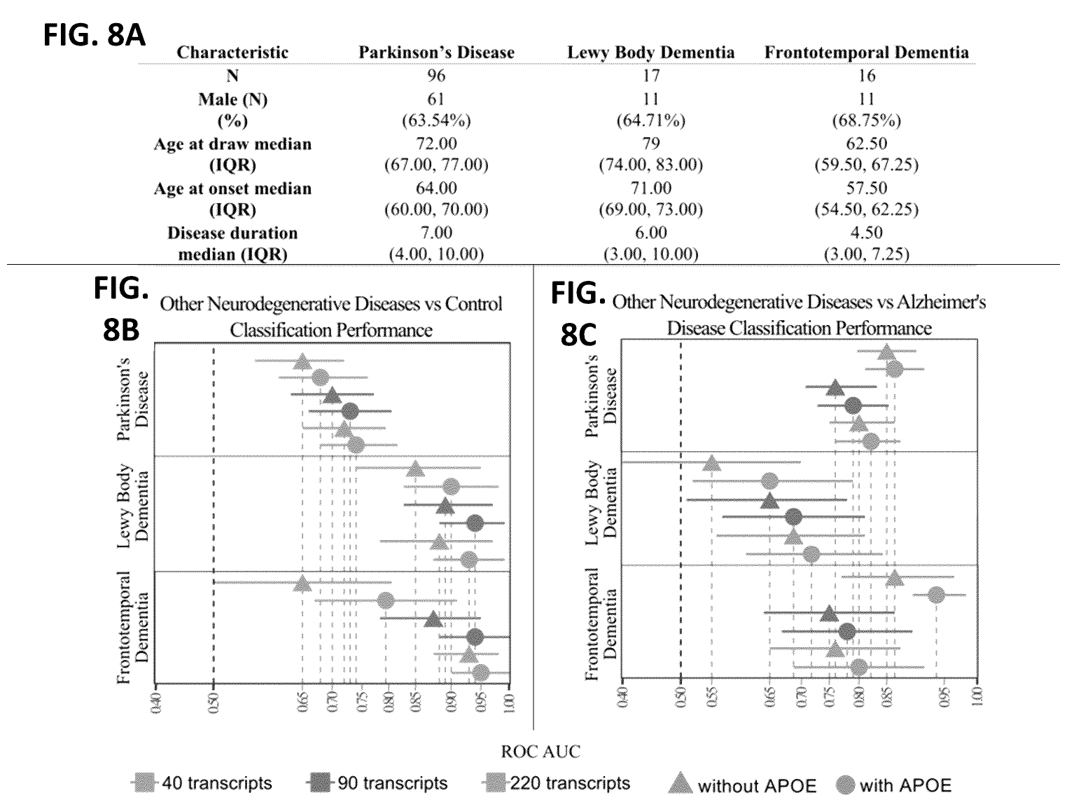

Lastly, we wanted to assess if the models were specific to AD. We evaluated the performance of our models in samples from Parkinson's disease (PD-n=96), Lewy body dementia (DLB-n-17) and Frontotemporal dementia (FTD-n=16) (A). We tested the specificity using two approaches, firstly, we asked if the models could correctly classify PD/DLB/FTD when compared to controls (FIG. 8B and Table 3), and secondly, if they could classify them when compared to AD (FIG. 8C and Table 7). The models had low predictive power to differentiate PD from controls (AUC<0.72), while the performance for FTD and DLB varied and depended on the number of transcripts (0.64<AUC<0.93), suggesting that the models are specific to AD, but we might be capturing the same biological process in diseases with high overlap like DLB and AD. In this case, the addition of APOE genotype did not decrease the predictive power, and this did not increase the specificity (FIG. 8B, Table 3). Similarly, when differentiating between AD and other neurodegenerative diseases, the models had high predictive power to differentiate PD (0.77<AUC<0.85) and FTD (0.75<AUC<0.86), but not as much for DLB (0.55<AUC<0.69). In contrast with the previous sections, the addition of APOE genotype improved the differentiation of AD from other neurodegenerative diseases with AUC>0.70 in all cases (FIGS. 8(B-C) and Table 7).

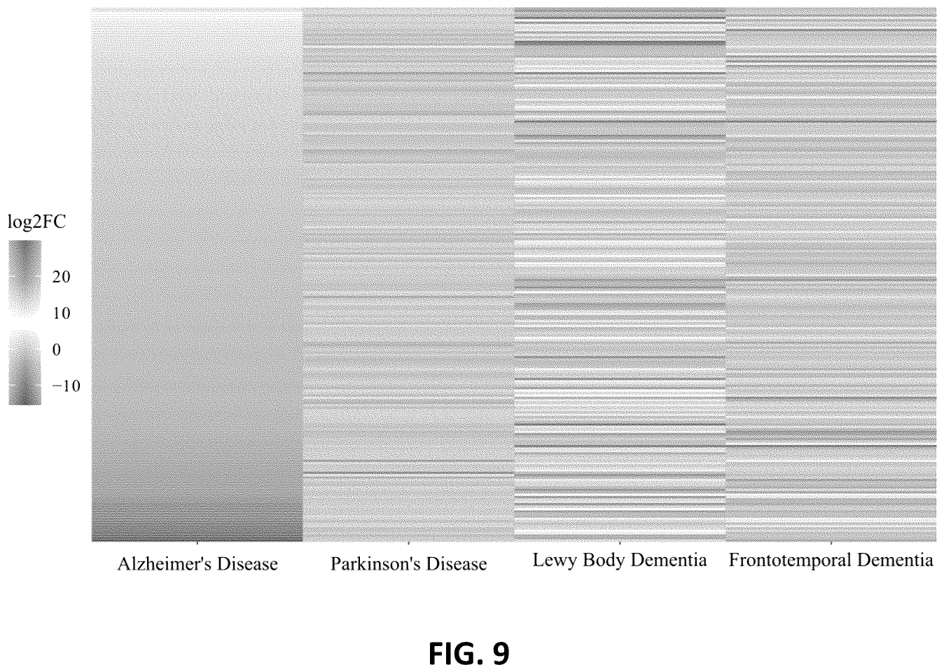

The model with 220 transcripts could differentiate PD from AD participants with an AUC of 0.81, whereas the differentiation from DLB or FTD (AUC<0.76) was less accurate. For the models including a smaller number of transcripts (90 and 40), they could differentiate AD from PD (AUC>0.81) and FTD (AUC>0.75), but not from DLB (AUC<0.65), suggesting that there are several transcripts that are commonly dysregulated in the two diseases and thus not useful for the differentiation. In fact, when we evaluated the expression patterns in each neurodegenerative disease compared to controls of all the transcripts included in the three models, we observed that the same transcripts were dysregulated in all three diseases, but in different directions when compared to controls. DLB was the one with the most striking differences in dysregulation for the transcripts selected in the predictive models (FIG. 9), suggesting that DLB have several genes commonly dysregulated with AD, but in different directions and proportions. In consequence, it is possible to hypothesize that DLB has, not only more clinical features shared with AD, but also more molecular pathways than those shared with PD or FTD, making it the most difficult to differentiate by the transcription models.

### Discussion

This is the first study using plasma cfRNA to create machine learning-based predictive models able to identify AD at the presymptomatic stages in two independent datasets. We identified transcripts in plasma that seem to recapitulate the changes taking place in the brain of AD participants, suggesting that changes taking place in the brain are leaking into the blood stream, most likely due to blood-brain barrier (BBB) breakdown. We have also built predictive models that correctly classify presymptomatic AD and control participants with a reasonable number of transcripts, and that showed high accuracy and specificity for AD. Given the reduced number of transcripts that the models include, they are potentially applicable to the clinical setting if further testing supports their beneficial use. Studies with larger sample sizes are needed to improve the performance of the predictive models, however, here, we are demonstrating for the first time that cfRNA not only can be used as an early predictive tool but also that it captures early pathological changes.

We have investigated the early changes in plasma using cfRNA quantification, and identified a significant overlap with those previously published. On top of that, we have also proven that early changes in cfRNA plasma might be originating in the brain since several transcripts are also DE in brains of individuals with AD. Additionally, we identified that the cfRNA dysregulated transcripts are part of co-expression modules already identified in the cortex of AD cases and enriched in GO terms associated with the brain such as synapse and postsynapse. We also found an association with oligodendrocytes and cytoskeleton for the identified transcripts. Cytoskeleton organization seems to play a key role in oligodendrocyte proliferation. For example, TRAK2, a DE transcript in plasma of presymptomatic AD participants, is associated with oligodendrocytes by participating in the regulation of the organization of the actin cytoskeleton and with mitochondrial transport, all processes that may be contributing to AD. Several studies suggest that there is an early breakdown of the BBB due to the initiation of the disease, fact that supports the central nervous system origin of our findings, and those from others.

To our knowledge, the study by Toden et al. was the only one that evaluated plasma cfRNA as AD biomarker. However, it has important differences with the present study that make our study design better suited to build predictive models. First, it did not include presymptomatic AD participants, thus their model with 1658 transcripts is only applicable to clinical phases. Second, they did not perform specificity analysis with other neurodegenerative diseases, in consequence, the specificity of the model is unknown. Third, they used the 1658 DE transcripts to build the model, which, given the elevated number of transcripts, makes it difficult to translate it to a clinical setting. On top of that, by using all the DE transcripts the model has the potential to be redundant, overfitted and thus, non-generalizable. We have taken a more conservative approach, by developing several predictive models, considerably simpler in terms of the number of transcripts, to understand how cfRNA behaves in the context of AD. We have also included different neurodegenerative diseases to calculate the specificity and acknowledge the known overlap across diseases. Finally, we have studied the correlation between CSF AD biomarkers and ATN criteria to compare with the tools used in real clinical settings.

To date, CSF biomarkers have proven to be the most effective approach to classifying AD. Combinations of CSF biomarkers classify clinical AD and controls with accuracies ranging from 0.6 to 0.95 depending on age. The most used and accurate CSF biomarker is the Aβ42/Aβ40 ratio which can correctly diagnose 82.8% of the screened AD patients. In addition, combinations of CSF showed also good accuracy in detecting incipient AD in participants with mild cognitive impairment (MCI). Current plasma biomarkers show accuracies similar to that of CSF. The combination of plasma Aβ42/Aβ40, age, and APOE ε4 status is highly correlated with amyloid PET positivity and thus it could be used to screen for individuals prior to lumbar puncture, PET scan, or further testing. The three cfRNA models yield similar accuracy to that of plasma Aβ42/Aβ40, without including other variables. In fact, we observe that the addition of APOE does not improve the models. The main advantage of cfRNA from the current protein measurements, it is its potential to be translated to a real-time PCR, which is a more cost-effective technique that can be implemented in all clinical settings, even in those that are remote. Additionally, given the independence from Aβ, cfRNA could be used to therapy monitoring when we evaluated Aβ protein-targeted drugs.

Predictive models built using machine learning approaches in neurodegenerative diseases tend to contain a large number of features, or contain only the identified DE genes, transcripts, or proteins. In here, we focused on models with a relatively low number of transcripts without compromising accuracy to maximize their potential to be translated to the clinic. Previous models using cfRNA reported an AUC of 0.83 for clinical AD; by substantially reducing the number of transcripts, we have increased the AUC up to 0.94 for presymptomatic AD and using only 220 transcripts. For clinical phases, the model with 220 genes showed a stable accuracy, outperforming the previously published. We also demonstrated that the cfRNA predictive model is specific to AD, since it is not able to predict PD, or FTD. Even though the model is able to predict DLB with a reasonable accuracy, we expect some degree of overlap in the prediction of these diseases due to the existing clinical and pathological overlap.

This study has several limitations. The sample size we reached for presymptomatic AD is rather limited. However, to the best of our knowledge, this is the largest sample of presymptomatic sporadic AD with clinical retrospective data there is. Due to the sample selection strategy, the samples have been stored in the freezer for long periods of time, which might affect our findings. Considering this, we have removed any transcript that showed selective degradation to minimize this effect. The use of RNA-seq techniques is very sensitive to bias, especially to using machine learning afterward. While the two datasets are methodologically independent according to the machine learning field, they originate from the same site, which adds to the potential bias effect of the present study. Nonetheless, for modeling purposes, the generation of two independent datasets and the use of mathematical approaches have mitigated the presence of potential methodological bias. Additionally, we have proposed a new approach to integrate RNAseq datasets applicable in large studies utilizing multiple datasets and different data types. Finally, larger sample sizes for all AD stages and other neurodegenerative diseases are needed to confirm our DE findings and generalize and improve the accuracy and specificity of the model. Nevertheless, we believe that this study serves as proof that cfRNA has the potential to detect changes related to AD pathobiology, even before the onset of symptoms.

Despite of the aforementioned limitations, in this study we were able to model and replicate in an independent dataset a predictor that can identify presymptomatic AD. On top of that, the predictor has been designed independently of Aβ42, which makes it an excellent candidate to monitor potential disease modifying therapies. The use of plasma cfRNA as biomarker is very advantageous due to its cost-effectiveness compared to current CSF and plasma measures and the fact that cfRNA models have the potential to be transformed into real-time PCR panels, therefore cfRNA could be smoothly implemented in the clinic without additional equipment or training, also in remote settings, something impossible with the current tools. Overall, we believe that further longitudinal studies with larger sample sizes are needed to confirm the use of cfRNA as a biomarker, but the current results show unprecedented potential.

### Material and Methods

Study Design

RNA was extracted from unfasted plasma samples from AD participants, controls and other neurodegenerative diseases from two independent cohorts, both from the Knight Alzheimer Disease Research Center (Knight-ADRC) and the Movement Disorders Clinic (MDC) at Washington University in Saint Louis. After library preparation, sequencing, and stringent quality control, we compared the presymptomatic AD participants to the controls from the discovery cohort to identify differentially expressed transcripts. We compared our results to those previously published, and to those identified to be differentially expressed in brain to understand the potential origin of the altered transcripts. Then we leveraged machine learning tools to build predictive models that differentiate between presymptomatic AD participants and controls with a scalable number of genes that were replicated in an independent cohort. Finally, we calculated the predictive value of the models in symptomatic AD (CDR®=0.5 and CDR®=1) to test if the models were useful in clinical stages of the diseases, and in other neurodegenerative diseases such as Parkinson's Disease (PD), Lewy Body Dementia (DLB), and Frontotemporal Dementia (FTD) to test their specificity to AD (FIG. 1A).

Study Participants

Plasma samples were obtained from the Knight-ADRC and the MDC at Washington University in Saint Louis repositories. These are deeply phenotyped cohorts, both clinically and molecularly with longitudinal data and samples available. We included 48 samples from healthy non-demented control participants, 67 samples from presymptomatic AD participants (Clinical Dementia Rating (64) (CDR®)=0 at draw and current clinical diagnostic of AD), 42 samples from early symptomatic AD participants (CDR®-0.5 at draw and current diagnostic of AD), and 50 samples from symptomatic AD (CDR®-1 at draw, diagnostic of AD at draw, and current diagnostic of AD) (FIGS. 1(A-B) and FIG. 6A). All AD participants were required to have evidence of Aβ deposition (CSF Aβ<500 ng/L), positive PET scan and/or evidence of clinical worsening measured by CDR® from the time at draw to the last clinical visit. For 71 participants, timely matched CSF biomarker measurements and time at draw are available. We also included participants from other neurodegenerative diseases: 17 DLB participants, 16 FTD participants, and 96 PD participants (FIG. 1A and FIG. 8A). AD, DLB, and FTD participants were diagnosed in accordance with clinical criteria that are embodied in the Uniform Data Set (UDS), the standard clinical data set that is collected in all participants who are enrolled in all of the 37-federally funded ADRCs. PD participants were clinically diagnosed according to the UK Brain Bank criteria. This research was conducted in accordance with the recommended protocols. Written informed consent was obtained from all participants or their family members. The Washington University in Saint Louis Institutional Review Board approved the study (IRB ID 201701124 and 202004010).

RNA Extraction and Sequencing

Non-fasted plasma samples are collected as part of the research protocol every two years for all participants. After whole blood is obtained, it is centrifuged within 20 minutes at 1500 rpm for 10 min to obtain plasma and stored at −80° C. until assayed. Selected plasma samples from study participants that meet the inclusion criteria were thawed on ice and centrifuged at 2000 rpm for 5 min prior to RNA extraction to avoid cell RNA contamination. Samples were processed in two batches. For the training batch (n=245), total plasma cfRNA was extracted from 0.5 mL of plasma using the Maxwell RSC miRNA from plasma or serum kit (Ambion) and ribodepleted (NEBNext rRNA Depletion Kit). For the testing batch (N=91), total cfRNA was extracted from 1 mL of plasma using the QIAmp Circulating Nucleic Acid kit (QIAGEN) followed by a DNAsel digestion (New England Biolabs). In both cases, libraries were generated using the NEBNext Ultra II Directional RNA Library Prep Kit for Illumina (New England Biolabs) using 1 ng of RNA as input. Libraries were cleaned for possible adapter dimers. We targeted 40 million 100 base pair single-end reads for each sample using an Illumina NovaSeq 6000 for the training batch, and 15 million 100 base pair reads single-end reads Illumina HiSeq 2500 for the testing.

Data Processing and Quality Control

We used FastQC (v0.11.7) to evaluate the sequencing quality of each sample. Then, we used STAR (v2.7.1a) to obtain the BAM files and align them to the human reference genome GRCh38. After that, we used PICARD (v2.26) and SamTools to assess the quality of the sequences and the alignment. Finally, we used Salmon (v0.11.3) to quantify the expression of transcripts. MultiQC (v1.9) was used to gather quality control measures. We applied stringent quality control (QC). Briefly, after removing all transcripts with less than ten reads in more than 90% of the individuals, we calculated transcriptome Principal Component Analysis (PCA) and screen for correlation with technical and methodological variables to detect potential biases. We observed a strong correlation with total reads and coding bases; thus, we removed samples with less than 10% of coding bases and less than 1000000 total reads that were part of the same sequencing round. We also removed outlier samples based on transcriptome PCA and Cook's distances. Plasma samples have been stored for long periods of time prior to usage (up to 20 years), consequently, to address degradation, we used DESeq2 (v1.22.2) to find transcripts associated with storage time in control participants. All transcripts nominally (p<0.05) associated were removed from the analyses (n=2,580). Finally, we used DESeq2 to adjust for library complexity and normalize the counts using log transformation for the remaining transcripts (n=19,830) and obtained the final population we used for the present analyses.

Differential Expression Analyses and Pathway Analyses

Differential expression (DE) analyses were performed using DESeq2. All analyses were adjusted by gender and age at draw. We used the Benjamini-Hochberg correction (FDR) to correct for multiple testing. FDR p-values below 0.05 were considered significant. To replicate our findings, we used the DE transcripts identified in cfRNA by Toden et al. Additionally, to evaluate if those transcripts were also DE in the brains of AD participants, we used an in-house RNAseq dataset from brains of participants of the Knight-ADRC. To functionally characterize the DE transcripts we carried out gene-ontology enrichment analysis using the ToppGene Suite, disease pathway overlap analysis using KEGG, and gene co-expression network analysis using CoExp Web with the Religious Orders Study and Rush Memory and Aging Project (ROSMAP) data (72) as a background matrix. For these analyses, we also used FDR to correct for multiple testing. Corrected p-values below 0.05 were considered as significant.

Predictive Models Construction and Evaluation

We designed a specific machine learning pipeline to produce a suitable classifier to identify presymptomatic AD cases based on gene expression and using two independent datasets. Briefly, we scaled the two datasets using z-scores and generate a linear model comparing the 47 presymptomatic AD cases and the 26 controls in the training dataset. We did the same for the testing dataset (20 presymptomatic AD cases and 22 controls). We kept only those transcripts that had the same direction of effect regardless of the p-value. With the remaining transcripts, we calculated the Kullback-Leibler divergence (KLD) between the training and the testing dataset for each transcript, using the entropy R package (v1.3.1). We used the absolute value of the effect size from the linear model and the KLD value to rank the transcripts. Then we generate subsets of 40, 65, 90, 120, 150, 180, 220 and 250 transcripts. For each subset, we generated a model using KLD thresholds between 0.06 and 0.36 by increments of 0.02 and the R package glmnet (v2.0.16)(FIG. 3). We trained a total of 272 L2 regularization linear models. We selected the best based on the cross-validation error estimated produced by the algorithm on the training dataset (FIG. 4).

To understand the biology associated to the predictive models, we performed pathway analyses following the approach described above. To add robustness to per-model pathway analyses, each transcript set was expanded to include transcripts significantly correlated (p<0.05 and r>0.95) to transcripts in each of the predictive models, respectively.

Assessment of AD Risk Factors

Brain amyloidosis is the biomarker of reference for AD. To assess if the predictive models were correlated with brain amyloidosis, along with other known AD risk factors, we used the Spearman correlation between the estimated risk provided by the classifier and the CSF levels of Aβ42, tau, p-tau. We only included those individuals with CSF measurements available within seven years before or after the draw date (n=72). Additional to the correlation, we also used the CSF values to classify the participants using the ATN criteria, and then tested the performance of the models using the ATN criteria as outcome. We tested the performance of the cfRNA transcriptome models to differentiate between A positivity, and AT positivity. No data was available for the N criteria for these samples.

Sensitivity to AD Stages and Specificity Evaluation

To assess the AD continuum, we calculated the performance of the predictive models in the combined early symptomatic (CDR®=0.5) and symptomatic AD (CDR®=1) participants (FIG. 6A). We scaled the gene counts to the range of the training population by computing the z-score using the mean and standard deviation from the training population. Then, we calculated the risk score for each individual using the L2 regularization formula. Scores higher than 0.50 were considered cases. To calculate the ROC curve, we compared the predicted status to the true status for each group.

Due to the clinical and pathological overlap across neurodegenerative diseases, one of the challenges in the development of biomarkers for neurodegeneration is specificity to disease. To evaluate the performance of the predictive models in the context of other neurodegenerative diseases, we calculated the predictive risk value in 96 PD individuals, 16 DLB and 17 FTD (FIG. 8A) and computed the ROC curves as described above. Additionally, we also calculated the ROC curve using AD samples instead of controls as the comparison group. Finally, we evaluated if the model showed any improvement when adding APOE genotype to the cfRNA predictor. APOE is the most important genetic risk factor. Thus, to understand if the effect of APOE was captured by the predictor, we included the APOE genotype in the model coded by two variables representing the number of ε2 alleles and ε4 alleles.

Plasma cfRNA is a powerful tool for preclinical AD screening, prediction, and detection. As disclosed herein, it is a minimally-invasive biomarker with an accuracy higher than or comparable to current CSF biomarkers, and is AD-specific.

### Tables

TABLE 1
Summary results for the plasma cell-free transcripts differentially
expressed in the presymptomatic individuals and controls comparison.

| | | | | | | | Brain | |
|---|---|---|---|---|---|---|---|---|
| | | | | Toden et | | | Cortex | KEGG |
| | log2Fold | | | al. (17) | Brain (S1) | | Expression | AD |
| Gene | Change | IfcSE | P Value | Reported | P Value | Log2FC | (GTEx) | Pathway |
| ABI3 | 0.857 | 0.205 | 2.82E−05 | | 1.66E−01 | −0.293 | Yes | No |
| AEBP2 | −0.863 | 0.267 | 1.22E−03 | Down- | 2.65E−02 | 0.140 | Yes | No |
| | | | | regulated | | | | |
| AIF1L | 0.999 | 0.182 | 4.34E−08 | | 6.39E−02 | −0.372 | Yes | No |
| AKT3 | −0.741 | 0.195 | 1.50E−04 | | 3.29E−01 | 0.078 | Yes | Yes |
| ANKRD12 | −1.015 | 0.242 | 2.71E−05 | Down- | 4.48E−01 | −0.037 | Yes | No |
| | | | | regulated | | | | |
| ANP32A | 0.558 | 0.140 | 6.61E−05 | | 1.46E−02 | −0.225 | Yes | No |
| ARHGAP27 | 0.923 | 0.281 | 1.02E−03 | | 3.50E−01 | −0.128 | Yes | No |
| ARHGDIA | 0.552 | 0.165 | 8.29E−04 | | 1.46E−01 | −0.122 | Yes | No |
| ARHGEF15 | 1.631 | 0.355 | 4.30E−06 | | 1.76E−01 | −0.248 | Yes | No |
| ARRDC2 | 0.743 | 0.200 | 1.99E−04 | | 8.10E−01 | −0.053 | Yes | No |
| ASAH1 | −0.928 | 0.256 | 2.85E−04 | | 3.56E−03 | −0.252 | Yes | No |
| ATP1A1 | 0.795 | 0.222 | 3.43E−04 | | 1.15E−02 | 0.464 | Yes | No |
| ATP5PD | −0.855 | 0.243 | 4.28E−04 | | 7.03E−01 | −0.048 | No | Yes |
| AXIN1 | 1.099 | 0.325 | 7.19E−04 | | 5.69E−01 | 0.053 | Yes | Yes |
| BCL6B | 0.878 | 0.215 | 4.44E−05 | | 1.27E−01 | 0.398 | Yes | No |
| BLOC1S6 | −0.764 | 0.235 | 1.13E−03 | | 7.05E−02 | 0.168 | Yes | No |
| BMPR2 | −1.005 | 0.319 | 1.65E−03 | Down- | 3.78E−02 | 0.232 | Yes | No |
| | | | | regulated | | | | |
| BRD2 | 0.596 | 0.184 | 1.21E−03 | | 7.49E−01 | −0.019 | Yes | No |
| CA1 | 0.827 | 0.237 | 4.79E−04 | | 8.68E−02 | −1.201 | Yes | No |
| CAPRIN1 | 0.633 | 0.166 | 1.33E−04 | | 9.46E−01 | −0.004 | Yes | No |
| CARD11 | 0.895 | 0.265 | 7.45E−04 | | 5.48E−05 | −0.918 | Yes | No |
| CAVIN1 | 0.793 | 0.174 | 5.17E−06 | | 6.62E−01 | −0.079 | No | No |
| CCDC124 | 0.828 | 0.262 | 1.61E−03 | | 2.53E−01 | 0.084 | Yes | No |
| CCDC126 | −0.913 | 0.245 | 1.98E−04 | | 6.63E−01 | 0.044 | Yes | No |
| CENPB | 0.633 | 0.137 | 4.03E−06 | | 3.76E−01 | −0.138 | Yes | No |
| CENPBD1P1 | −2.219 | 0.593 | 1.85E−04 | | 2.79E−02 | 0.233 | Yes | No |
| CES2 | 1.120 | 0.341 | 1.01E−03 | | 5.43E−01 | −0.062 | Yes | No |
| CIRBP | 0.484 | 0.149 | 1.20E−03 | | 3.97E−02 | −0.424 | Yes | No |
| CNP | 0.861 | 0.263 | 1.07E−03 | | 4.32E−02 | −0.498 | Yes | No |
| CRIP2 | 0.800 | 0.220 | 2.83E−04 | | 1.14E−01 | 0.188 | Yes | No |
| CYTH1 | 0.825 | 0.179 | 4.00E−06 | Up- | 5.29E−02 | −0.249 | Yes | No |
| | | | | regulated | | | | |
| DAD1 | −1.098 | 0.290 | 1.57E−04 | Down- | 2.78E−01 | −0.125 | Yes | No |
| | | | | regulated | | | | |
| DCTN1 | 0.841 | 0.232 | 2.87E−04 | | 4.04E−01 | 0.062 | Yes | No |
| DDX54 | 0.921 | 0.291 | 1.57E−03 | Up- | 1.48E−01 | −0.104 | Yes | No |
| | | | | regulated | | | | |
| DOCK2 | 0.561 | 0.182 | 2.01E−03 | Up- | 7.38E−02 | −0.326 | Yes | No |
| | | | | regulated | | | | |
| DPYSL2 | 0.686 | 0.169 | 5.12E−05 | | 6.81E−01 | −0.029 | Yes | No |
| DYNC1LI2 | 1.123 | 0.219 | 2.91E−07 | | 4.33E−01 | −0.094 | Yes | No |
| DYRK1A | −0.689 | 0.222 | 1.90E−03 | Down- | 7.20E−03 | 0.138 | Yes | No |
| | | | | regulated | | | | |
| EFCAB13 | −1.210 | 0.326 | 2.05E−04 | | 7.34E−01 | −0.071 | Yes | No |
| EHD2 | 1.316 | 0.393 | 8.14E−04 | | 4.57E−01 | 0.132 | Yes | No |
| EIF3A | 0.473 | 0.152 | 1.92E−03 | | 3.22E−01 | −0.053 | Yes | No |
| EIF3L | 0.388 | 0.125 | 1.91E−03 | | 1.64E−01 | −0.133 | Yes | No |
| EPAS1 | 0.890 | 0.223 | 6.53E−05 | | 4.56E−02 | −0.319 | Yes | No |
| EPB42 | 1.246 | 0.333 | 1.86E−04 | | 8.74E−01 | −0.166 | Yes | No |
| ERBIN | −0.522 | 0.164 | 1.46E−03 | Down- | 4.68E−01 | −0.125 | Yes | No |
| | | | | regulated | | | | |
| ERG | 0.951 | 0.180 | 1.25E−07 | | 9.67E−01 | −0.006 | Yes | No |
| FAM107A | 0.832 | 0.193 | 1.58E−05 | | 2.81E−01 | 0.195 | Yes | No |
| FAM219A | 0.903 | 0.257 | 4.47E−04 | | 1.83E−02 | 0.160 | Yes | No |
| FANCL | −0.806 | 0.259 | 1.87E−03 | Down- | 6.20E−02 | −0.240 | Yes | No |
| | | | | regulated | | | | |
| FBRS | 1.001 | 0.282 | 3.75E−04 | | 4.89E−01 | −0.079 | Yes | No |
| FCHSD2 | −0.837 | 0.271 | 2.05E−03 | Down- | 1.29E−01 | −0.141 | Yes | No |
| | | | | regulated | | | | |
| FGD3 | 1.510 | 0.410 | 2.32E−04 | | 2.30E−01 | −0.266 | Yes | No |
| FGD5 | 1.125 | 0.282 | 6.74E−05 | | 4.59E−02 | 0.291 | Yes | No |
| FHDC1 | 1.562 | 0.283 | 3.27E−08 | | 7.06E−01 | −0.072 | Yes | No |
| FP236383.2 | 2.212 | 0.432 | 3.04E−07 | | 6.39E−02 | | No | No |
| FP236383.3 | 1.916 | 0.474 | 5.37E−05 | | 3.29E−01 | −0.501 | No | No |
| FP671120.3 | 2.476 | 0.449 | 3.56E−08 | | 4.48E−01 | | No | No |
| GAB2 | 0.960 | 0.257 | 1.86E−04 | | 1.46E−02 | −0.306 | Yes | No |
| GLS | −0.775 | 0.239 | 1.19E−03 | | 3.50E−01 | 0.398 | Yes | No |
| H19 | 0.654 | 0.204 | 1.36E−03 | | 9.48E−03 | 1.039 | Yes | No |
| H3F3A | −0.876 | 0.196 | 8.11E−06 | | | | Yes | No |
| HBD | 0.832 | 0.267 | 1.86E−03 | | 6.45E−01 | −0.393 | Yes | No |
| HECW2 | 1.463 | 0.327 | 7.63E−06 | | 3.99E−01 | 0.072 | Yes | No |
| HEG1 | 1.124 | 0.364 | 2.02E−03 | | 3.99E−01 | −0.137 | Yes | No |
| HIPK3 | 0.515 | 0.153 | 7.82E−04 | Down- | 1.38E−02 | −0.189 | Yes | No |
| | | | | regulated | | | | |
| HIST1H3H | −1.251 | 0.296 | 2.40E−05 | Down- | | | Yes | No |
| | | | | regulated | | | | |
| HIST2H2BE | −0.783 | 0.250 | 1.78E−03 | Down- | | | Yes | No |
| | | | | regulated | | | | |
| HLA-B | −0.886 | 0.226 | 9.02E−05 | | 6.50E−02 | −0.298 | Yes | No |
| HNRNPM | 0.468 | 0.121 | 1.07E−04 | | 4.99E−01 | −0.040 | Yes | No |
| HSP90AB1 | 0.275 | 0.083 | 9.57E−04 | Up- | 5.37E−01 | 0.081 | Yes | No |
| | | | | regulated | | | | |
| HSPA12B | 0.751 | 0.245 | 2.16E−03 | | 3.68E−01 | 0.165 | Yes | No |
| IGFBP4 | 0.754 | 0.213 | 3.93E−04 | | 5.63E−01 | 0.113 | Yes | No |
| IGFBP5 | 0.680 | 0.220 | 2.01E−03 | | 6.46E−01 | −0.095 | Yes | No |
| IPO5 | 0.629 | 0.196 | 1.35E−03 | Up- | 1.48E−01 | 0.099 | Yes | No |
| | | | | regulated | | | | |
| IRAK3 | −0.687 | 0.208 | 9.65E−04 | | 3.53E−01 | −0.182 | Yes | No |
| JCAD | 1.046 | 0.183 | 1.13E−08 | | 4.40E−02 | 0.234 | No | No |
| KANK2 | 1.230 | 0.353 | 4.99E−04 | | 5.13E−01 | 0.120 | Yes | No |
| KAT6A | 0.672 | 0.188 | 3.48E−04 | | 2.16E−02 | −0.203 | Yes | No |
| KIF1C | 0.811 | 0.193 | 2.58E−05 | | 5.06E−01 | −0.137 | Yes | No |
| KIF26A | 1.242 | 0.234 | 1.17E−07 | | 2.76E−01 | 0.231 | Yes | No |
| KLF13 | 0.506 | 0.159 | 1.48E−03 | | 1.97E−01 | −0.115 | Yes | No |
| LDLRAD4 | 1.091 | 0.327 | 8.35E−04 | | 3.89E−02 | −0.200 | Yes | No |
| LGALS1 | 0.761 | 0.214 | 3.64E−04 | Up- | 5.64E−01 | 0.065 | Yes | No |
| | | | | regulated | | | | |
| MALAT1 | −1.410 | 0.327 | 1.57E−05 | | 9.36E−01 | −0.010 | Yes | No |
| MAN1A2 | −0.742 | 0.241 | 2.10E−03 | Down- | 7.81E−01 | 0.019 | Yes | No |
| | | | | regulated | | | | |
| MAN2A1 | −0.847 | 0.221 | 1.23E−04 | Down- | 3.54E−01 | −0.217 | Yes | No |
| | | | | regulated | | | | |
| MAP4K4 | 0.629 | 0.161 | 9.00E−05 | | 4.57E−01 | −0.145 | Yes | No |
| MBD3 | 0.800 | 0.258 | 1.91E−03 | | 1.58E−02 | 0.285 | Yes | No |
| MBOAT2 | −1.435 | 0.322 | 8.19E−06 | Down- | 4.10E−04 | −0.349 | Yes | No |
| | | | | regulated | | | | |
| MKLN1 | −0.941 | 0.284 | 9.23E−04 | | 3.67E−01 | −0.062 | Yes | No |
| MYL6 | −0.768 | 0.165 | 3.33E−06 | | 1.19E−02 | −0.206 | Yes | No |
| NCKAP5L | 1.145 | 0.333 | 5.86E−04 | | 4.03E−01 | −0.093 | Yes | No |
| NDUFA4 | −0.693 | 0.217 | 1.40E−03 | | 6.11E−01 | 0.092 | Yes | Yes |
| NDUFA5 | −1.047 | 0.329 | 1.45E−03 | | 7.57E−01 | 0.050 | Yes | Yes |
| NDUFA6 | −1.008 | 0.303 | 8.77E−04 | Down- | 8.15E−01 | −0.027 | Yes | Yes |
| | | | | regulated | | | | |
| NES | 0.886 | 0.203 | 1.22E−05 | | 1.66E−01 | −0.206 | Yes | No |
| NFE2L1 | 1.015 | 0.259 | 8.68E−05 | | 4.83E−02 | 0.123 | Yes | No |
| NOL4L | −1.291 | 0.328 | 8.19E−05 | | 3.23E−01 | −0.103 | Yes | No |
| NOMO2 | −1.583 | 0.476 | 8.70E−04 | | 2.10E−02 | 0.292 | Yes | No |
| NONO | 0.530 | 0.157 | 7.61E−04 | | 2.29E−03 | −0.137 | Yes | No |
| NRIP1 | −1.465 | 0.405 | 2.96E−04 | Down- | 1.50E−01 | 0.115 | Yes | No |
| | | | | regulated | | | | |
| OAZ1 | −0.457 | 0.127 | 3.04E−04 | | 3.02E−01 | 0.089 | Yes | No |
| PABPC4 | 0.655 | 0.176 | 2.03E−04 | | 3.33E−01 | −0.061 | Yes | No |
| PAFAH2 | −1.893 | 0.531 | 3.67E−04 | | 1.61E−03 | −0.381 | Yes | No |
| PARK7 | −0.484 | 0.156 | 2.00E−03 | | 8.03E−01 | 0.022 | Yes | No |
| PCDHAC2 | 5.854 | 1.896 | 2.01E−03 | | 6.51E−01 | 0.071 | Yes | No |
| PDCD11 | 1.570 | 0.440 | 3.58E−04 | | 7.18E−01 | −0.019 | Yes | No |
| PGLS | 0.492 | 0.140 | 4.32E−04 | | 1.67E−01 | −0.152 | Yes | No |
| PIK3R5 | 1.055 | 0.292 | 2.99E−04 | | 9.21E−01 | −0.021 | Yes | No |
| PITPNM3 | 1.355 | 0.339 | 6.26E−05 | | 2.36E−01 | 0.230 | Yes | No |
| PKP4 | 0.692 | 0.224 | 2.02E−03 | | 1.88E−01 | −0.230 | Yes | No |
| PLCB3 | 1.417 | 0.403 | 4.37E−04 | | 7.77E−01 | −0.043 | Yes | Yes |
| POC1B | −1.022 | 0.332 | 2.04E−03 | Down- | 2.68E−02 | −0.291 | Yes | No |
| | | | | regulated | | | | |
| PPFIBP1 | 0.933 | 0.245 | 1.36E−04 | | 8.34E−05 | 0.335 | Yes | No |
| PPM1F | 0.885 | 0.236 | 1.78E−04 | | 6.64E−02 | 0.155 | Yes | No |
| PRPF8 | 0.890 | 0.192 | 3.46E−06 | | 9.14E−01 | 0.005 | Yes | No |
| PTPN22 | −0.555 | 0.163 | 6.57E−04 | | 9.81E−01 | 0.006 | Yes | No |
| PURA | 0.639 | 0.201 | 1.46E−03 | | 4.68E−02 | 0.122 | Yes | No |
| RAD54L2 | 1.121 | 0.280 | 6.29E−05 | | 9.98E−01 | 0.000 | Yes | No |
| RARS | −0.640 | 0.183 | 4.70E−04 | | | | Yes | No |
| RBL1 | −0.925 | 0.301 | 2.08E−03 | | 1.45E−01 | −0.176 | Yes | No |
| RBL2 | 0.504 | 0.143 | 4.04E−04 | | 5.28E−02 | −0.162 | Yes | No |
| RBM8A | 0.610 | 0.198 | 2.04E−03 | | 1.38E−02 | −0.147 | Yes | No |
| RBX1 | −1.050 | 0.238 | 9.91E−06 | Down- | 3.13E−01 | −0.094 | Yes | No |
| | | | | regulated | | | | |
| RELA | 1.157 | 0.333 | 5.04E−04 | | 6.53E−01 | −0.075 | Yes | Yes |
| REPIN1 | 0.772 | 0.240 | 1.31E−03 | | 9.57E−01 | 0.005 | Yes | No |
| REPS2 | −1.584 | 0.415 | 1.32E−04 | Down- | 3.69E−02 | 0.282 | Yes | No |
| | | | | regulated | | | | |
| RHOBTB3 | −1.193 | 0.327 | 2.65E−04 | Down- | 4.98E−02 | −0.260 | Yes | No |
| | | | | regulated | | | | |
| RN7SL116P | −1.513 | 0.471 | 1.31E−03 | | 7.01E−01 | 0.151 | No | No |
| RN7SL126P | −1.543 | 0.467 | 9.56E−04 | | | | No | No |
| RN7SL151P | −1.535 | 0.389 | 7.87E−05 | | 9.85E−01 | −0.113 | No | No |
| RN7SL296P | −1.707 | 0.441 | 1.08E−04 | | | | No | No |
| RN7SL3 | −0.582 | 0.168 | 5.22E−04 | | 5.02E−01 | 0.168 | Yes | No |
| RN7SL396P | −1.696 | 0.479 | 4.00E−04 | | 9.80E−01 | 0.153 | No | No |
| RN7SL480P | −1.457 | 0.438 | 8.86E−04 | | | | No | No |
| RN7SL4P | −2.071 | 0.441 | 2.64E−06 | | 9.52E−01 | −0.015 | Yes | No |
| RN7SL564P | −1.430 | 0.414 | 5.46E−04 | | 9.85E−01 | 0.114 | No | No |
| RNF169 | 1.037 | 0.301 | 5.80E−04 | | 8.56E−01 | −0.017 | Yes | No |
| RNF24 | −1.234 | 0.387 | 1.41E−03 | Down- | 1.11E−02 | 0.291 | Yes | No |
| | | | | regulated | | | | |
| RUNX1 | −1.399 | 0.448 | 1.79E−03 | Down- | 4.42E−02 | −0.405 | Yes | No |
| | | | | regulated | | | | |
| RXRA | 0.473 | 0.145 | 1.14E−03 | | 9.88E−01 | −0.002 | Yes | No |
| S100A9 | −0.602 | 0.180 | 8.52E−04 | | 5.49E−01 | 0.298 | Yes | No |
| SAMD4A | 0.946 | 0.285 | 9.06E−04 | | 8.50E−01 | −0.035 | Yes | No |
| SBF1 | 0.835 | 0.244 | 6.14E−04 | | 7.52E−01 | 0.023 | Yes | No |
| SCRN1 | 0.758 | 0.244 | 1.92E−03 | | 1.22E−01 | 0.090 | Yes | No |
| SERINC1 | 0.396 | 0.125 | 1.61E−03 | | 9.19E−01 | −0.012 | Yes | No |
| SF3B2 | 0.547 | 0.172 | 1.43E−03 | | 1.09E−01 | −0.122 | Yes | No |
| SH3PXD2A | 0.845 | 0.206 | 4.21E−05 | | 9.96E−01 | 0.001 | Yes | No |
| SLC9A3R2 | 0.859 | 0.169 | 4.01E−07 | | 3.05E−01 | 0.136 | Yes | No |
| SLC9A9 | −1.588 | 0.494 | 1.31E−03 | Down- | 4.06E−02 | −0.298 | Yes | No |
| | | | | regulated | | | | |
| SND1 | 0.787 | 0.186 | 2.20E−05 | Up- | 2.27E−03 | −0.175 | Yes | No |
| | | | | regulated | | | | |
| SNRNP200 | 0.521 | 0.150 | 5.29E−04 | | 3.27E−01 | −0.040 | Yes | No |
| SNRPN | −1.005 | 0.241 | 3.08E−05 | | 7.87E−02 | 0.236 | Yes | No |
| SNX1 | 0.949 | 0.275 | 5.45E−04 | | 9.66E−02 | −0.152 | Yes | No |
| SNX6 | −0.623 | 0.185 | 7.49E−04 | | 9.24E−02 | −0.203 | Yes | No |
| SOS1 | −0.736 | 0.238 | 2.00E−03 | Down- | 6.32E−01 | −0.033 | Yes | No |
| | | | | regulated | | | | |
| SPTAN1 | 1.096 | 0.237 | 3.71E−06 | | 2.26E−02 | 0.194 | Yes | No |
| SPTBN1 | 0.840 | 0.143 | 3.88E−09 | | 4.28E−01 | 0.045 | Yes | No |
| SRBD1 | −0.846 | 0.272 | 1.88E−03 | Down- | 1.39E−03 | −0.332 | Yes | No |
| | | | | regulated | | | | |
| SRRM1 | 0.659 | 0.209 | 1.58E−03 | | 1.41E−03 | −0.364 | Yes | No |
| SSR1 | −0.962 | 0.286 | 7.60E−04 | | 1.03E−01 | −0.099 | Yes | No |
| SUCLA2 | 1.808 | 0.528 | 6.18E−04 | | 4.06E−01 | 0.122 | Yes | No |
| SUCO | −0.943 | 0.303 | 1.85E−03 | | 4.41E−03 | −0.194 | Yes | No |
| SVIL | 1.759 | 0.506 | 5.02E−04 | | 4.98E−01 | 0.143 | Yes | No |
| SYNE1 | 0.687 | 0.201 | 6.21E−04 | | 6.06E−01 | 0.061 | Yes | No |
| SYNPO | 1.071 | 0.160 | 1.97E−11 | | 2.76E−03 | 0.595 | Yes | No |
| TBC1D14 | −0.823 | 0.248 | 9.11E−04 | Down- | 5.81E−01 | 0.031 | Yes | No |
| | | | | regulated | | | | |
| TM7SF3 | −1.035 | 0.311 | 8.93E−04 | Down- | 5.67E−01 | 0.030 | Yes | No |
| | | | | regulated | | | | |
| TMCC2 | 0.841 | 0.273 | 2.08E−03 | | 2.03E−01 | 0.130 | Yes | No |
| TMEM140 | −1.154 | 0.343 | 7.66E−04 | | 7.15E−02 | −0.497 | Yes | No |
| TMEM70 | −1.499 | 0.480 | 1.80E−03 | | 3.30E−01 | 0.144 | Yes | No |
| TMEM91 | −1.452 | 0.442 | 1.01E−03 | | 2.90E−01 | −0.119 | Yes | No |
| TRAK2 | 1.232 | 0.199 | 6.04E−10 | | 5.81E−01 | 0.067 | Yes | No |
| TSC22D3 | −0.561 | 0.158 | 3.65E−04 | | 3.82E−01 | 0.098 | Yes | No |
| TTLL5 | −1.631 | 0.513 | 1.48E−03 | Down- | 6.83E−01 | 0.027 | Yes | No |
| | | | | regulated | | | | |
| TXNL4B | −1.354 | 0.347 | 9.46E−05 | | 5.45E−01 | 0.057 | Yes | No |
| UBL5 | −0.593 | 0.153 | 1.08E−04 | | 4.25E−02 | −0.224 | Yes | No |
| UBR2 | −0.431 | 0.131 | 1.05E−03 | | 5.43E−01 | 0.031 | Yes | No |
| UQCRH | −0.517 | 0.154 | 7.70E−04 | | 4.14E−01 | 0.131 | Yes | Yes |
| VIM | 0.371 | 0.105 | 4.17E−04 | | 2.73E−01 | 0.222 | Yes | No |
| VIM-AS1 | −0.943 | 0.226 | 2.94E−05 | | 5.50E−01 | 0.256 | Yes | No |
| VMP1 | −0.661 | 0.211 | 1.72E−03 | | 1.58E−01 | −0.157 | Yes | No |
| WASHC4 | 0.705 | 0.208 | 6.86E−04 | | 7.30E−02 | −0.155 | Yes | No |
| ZC3H12C | −1.283 | 0.418 | 2.14E−03 | Down- | 3.46E−01 | −0.094 | Yes | No |
| | | | | regulated | | | | |
| ZEB1 | 0.814 | 0.193 | 2.36E−05 | | 2.65E−01 | 0.081 | Yes | No |
| ZFAND1 | −0.861 | 0.275 | 1.70E−03 | | 8.84E−01 | −0.017 | Yes | No |
| ZMYND8 | −1.662 | 0.367 | 6.08E−06 | Down- | 3.14E−03 | 0.305 | Yes | No |
| | | | | regulated | | | | |
| ZNF366 | 1.767 | 0.358 | 7.83E−07 | | 8.23E−01 | 0.042 | Yes | No |
| ZNF704 | 1.566 | 0.493 | 1.50E−03 | | 2.01E−01 | −0.128 | Yes | No |
| ZSWIM9 | 1.868 | 0.547 | 6.42E−04 | | 4.48E−01 | 0.111 | No | No |

TABLE 2
Performance of the three predictive models in presymptomatic
AD samples for the training and the testing datasets.

| | | Balanced | Cohen's | Sensi- | Speci- | |
|---|---|---|---|---|---|---|
| Model | Status | Accuracy | Kappa | tivity | ficity | AUC |
| 40 | Training | 0.957 | 0.885 | 1.000 | 0.915 | — |
| transcripts | Testing | 0.859 | 0.715 | 0.818 | 0.900 | 0.900 |
| model | | | | | | (0.819, |
| | | | | | | 0.981) |
| | Testing + | 0.809 | 0.618 | 0.818 | 0.800 | 0.934 |
| | APOE | | | | | (0.874, |
| | | | | | | 0.994) |
| 90 | Training | 0.926 | 0.803 | 1.000 | 0.851 | — |
| transcripts | Testing | 0.905 | 0.809 | 0.909 | 0.900 | 0.916 |
| model | | | | | | (0.834, |
| | | | | | | 0.998) |
| | Testing + | 0.857 | 0.714 | 0.864 | 0.850 | 0.950 |
| | APOE | | | | | (0.892, |
| | | | | | | 1.000) |
| 220 | Training | 0.936 | 0.830 | 1.000 | 0.872 | — |
| transcripts | Testing | 0.952 | 0.905 | 0.955 | 0.950 | 0.941 |
| model | | | | | | (0.871, |
| | | | | | | 1.000) |
| | Testing + | 0.902 | 0.808 | 0.955 | 0.850 | 0.975 |
| | APOE | | | | | (0.942, |
| | | | | | | 1.000) |

AUC = Area under the ROC Curve

TABLE 3
Performance of the three predictive models with and without
APOE genotype in predicting the AD continuum and other neurodegenerative
diseases when compared to control participants.

| | | | | Positive | Negative | |
|---|---|---|---|---|---|---|
| | | | | Predictive | Predictive | |
| Model | Status | Accuracy | Specificity | Value | Value | AUC |
| 40 | Early | 0.804 | 0.690 | 0.772 | 0.879 | 0.926 |
| transcripts | Symptomatic | | | | | (0.880, |
| model | AD | | | | | 0.971) |
| | Symptomatic | 0.828 | 0.740 | 0.772 | 0.902 | 0.908 |
| | AD | | | | | (0.860, |
| | | | | | | 0.955) |
| | Parkinson's | 0.613 | 0.309 | 0.404 | 0.613 | 0.650 |
| | Disease | | | | | (0.573, |
| | | | | | | 0.726) |
| | Lewy Body | 0.811 | 0.706 | 0.898 | 0.811 | 0.846 |
| | Dementia | | | | | (0.744, |
| | | | | | | 0.947) |
| | Frontotemporal | 0.615 | 0.313 | 0.800 | 0.615 | 0.638 |
| | Dementia | | | | | (0.488, |
| | | | | | | 0.788) |
| 90 | Early | 0.908 | 0.857 | 0.885 | 0.947 | 0.954 |
| transcripts | Symptomatic | | | | | (0.918, |
| model | AD | | | | | 0.990) |
| | Symptomatic | 0.869 | 0.780 | 0.807 | 0.951 | 0.938 |
| | AD | | | | | (0.901, |
| | | | | | | 0.975) |
| | Parkinson's | 0.681 | 0.404 | 0.451 | 0.950 | 0.703 |
| | Disease | | | | | (0.631, |
| | | | | | | 0.774) |
| | Lewy Body | 0.803 | 0.647 | 0.885 | 0.846 | 0.893 |
| | Dementia | | | | | (0.817, |
| | | | | | | 0.970) |
| | Frontotemporal | 0.760 | 0.563 | 0.868 | 0.818 | 0.866 |
| | Dementia | | | | | (0.782, |
| | | | | | | 0.950) |
| 220 | Early | 0.942 | 0.905 | 0.922 | 0.974 | 0.976 |
| transcripts | Symptomatic | | | | | (0.948, |
| model | AD | | | | | 1.000) |
| | Symptomatic | 0.860 | 0.740 | 0.783 | 0.974 | 0.960 |
| | AD | | | | | (0.930, |
| | | | | | | 0.990) |
| | Parkinson's | 0.692 | 0.404 | 0.456 | 0.974 | 0.719 |
| | Disease | | | | | (0.650, |
| | | | | | | 0.790) |
| | Lewy Body | 0.813 | 0.647 | 0.887 | 0.917 | 0.875 |
| | Dementia | | | | | (0.778 |
| | | | | | | 0.972) |
| | Frontotemporal | 0.833 | 0.688 | 0.904 | 0.917 | 0.928 |
| | Dementia | | | | | (0.875 |
| | | | | | | 0.982) |
| 40 | Early | 0.887 | 0.857 | 0.880 | 0.900 | 0.958 |
| transcripts | Symptomatic | | | | | (0.929, |
| model & | AD | | | | | 0.988) |
| APOE | | | | | | |
| genotype | Symptomatic | 0.928 | 0.940 | 0.936 | 0.922 | 0.956 |
| | AD | | | | | (0.923, |
| | | | | | | 0.990) |
| | Parkinson's | 0.582 | 0.894 | 0.565 | 0.706 | 0.685 |
| | Disease | | | | | (0.608, |
| | | | | | | 0.761) |
| | Lewy Body | 0.811 | 0.706 | 0.898 | 0.750 | 0.906 |
| | Dementia | | | | | (0.829, |
| | | | | | | 0.982) |
| | Frontotemporal | 0.719 | 0.563 | 0.857 | 0.600 | 0.781 |
| | Dementia | | | | | (0.660 |
| | | | | | | 0.903) |
| 90 | Early | 0.933 | 0.929 | 0.938 | 0.929 | 0.983 |
| transcripts | Symptomatic | | | | | (0.965 |
| model & | AD | | | | | 1.000) |
| APOE | Symptomatic | 0.918 | 0.920 | 0.917 | 0.920 | 0.962 |
| genotype | AD | | | | | (0.932 |
| | | | | | | 0.992) |
| | Parkinson's | 0.603 | 0.851 | 0.548 | 0.721 | 0.728 |
| | Disease | | | | | (0.659, |
| | | | | | | 0.798) |
| | Lewy Body | 0.792 | 0.647 | 0.882 | 0.786 | 0.936 |
| | Dementia | | | | | (0.883, |
| | | | | | | 0.989) |
| | Frontotemporal | 0.844 | 0.750 | 0.918 | 0.800 | 0.939 |
| | Dementia | | | | | (0.877, |
| | | | | | | 1.000) |
| 220 | Early | 0.967 | 0.976 | 0.979 | 0.953 | 0.991 |
| transcripts | Symptomatic | | | | | (0.981, |
| model & | AD | | | | | 1.000) |
| APOE | Symptomatic | 0.929 | 0.920 | 0.918 | 0.939 | 0.972 |
| genotype | AD | | | | | (0.947, |
| | | | | | | 0.997) |
| | Parkinson's | 0.649 | 0.819 | 0.575 | 0.755 | 0.744 |
| | Disease | | | | | (0.676, |
| | | | | | | 0.813) |
| | Lewy Body | 0.862 | 0.765 | 0.920 | 0.867 | 0.928 |
| | Dementia | | | | | (0.87, |
| | | | | | | 0.986) |
| | Frontotempor | 0.885 | 0.813 | 0.939 | 0.867 | 0.951 |
| | al Dementia | | | | | (0.902, |
| | | | | | | 0.999) |

AD = Alzheimer's Disease;
AUC = Area under the ROC Curve

TABLE 4
Performance of the three predictive models when classifying the
samples using the ATN criteria. We only included n = 71
samples with CSF measurements available at the time of blood draw.

| Model | | Status | AUC |
|---|---|---|---|
| 40 | without | AT⁻ vs AT⁺ | 0.80 |
| transcripts | APOE | | (0.64-0.96) |
| model | | A⁻ vs A⁺ | 0.86 |
| | | | (0.78-0.95) |
| | with | AT⁻ vs AT⁺ | 0.86 |
| | APOE | | (0.73-0.98) |
| | | A⁻ vs A⁺ | 0.86 |
| | | | (0.77-0.95) |
| 90 | without | AT⁻ vs AT⁺ | 0.88 |
| transcripts | APOE | | (0.75-1.00) |
| model | | A⁻ vs A⁺ | 0.88 |
| | | | (0.80-0.97) |
| | with | AT⁻ vs AT⁺ | 0.90 |
| | APOE | | (0.78-1.00) |
| | | A⁻ vs A⁺ | 0.88 |
| | | | (0.79-0.96) |
| 220 | without | AT⁻ vs AT⁺ | 0.88 |
| transcripts | APOE | | (0.73-1.00) |
| model | | A⁻ vs A⁺ | 0.89 |
| | | | (0.81-0.97) |
| | with | AT⁻ vs AT⁺ | 0.90 |
| | APOE | | (0.78-1.00) |
| | | A⁻ vs A⁺ | 0.88 |
| | | | (0.79-0.96) |

AUC = Area under the ROC Curve

TABLE 5
GO terms enriched in the expanded model pathway analyses for 40, 90 and 220 transcript models

| | | 40 transcripts | | 90 transcripts | | 220 transcripts | |
|---|---|---|---|---|---|---|---|
| | | model | | model | | model | |
| GO term | | Gene | | Gene | | Gene | |
| ID | GO term description | ratio | p-value | ratio | p-value | ratio | p-value |
| GO: 0000027 | ribosomal large subunit assembly | 6/810 | 6.24E−04 | 6/941 | 1.36E−03 | — | >0.05 |
| GO: 0000079 | regulation of cyclin-dependent | 12/810 | 3.35E−03 | — | >0.05 | — | >0.05 |
| | protein serine/threonine kinase | | | | | | |
| | activity | | | | | | |
| GO: 0000082 | G1/S transition of mitotic cell cycle | 25/810 | 1.30E−04 | 27/941 | 2.28E−04 | — | >0.05 |
| GO: 0000164 | protein phosphatase type 1 | 5/820 | 9.89E−04 | 5/959 | 1.99E−03 | — | >0.05 |
| | complex | | | | | | |
| GO: 0000220 | vacuolar proton-transporting V-type | — | >0.05 | 3/959 | 1.14E−02 | — | >0.05 |
| | ATPase, V0 domain | | | | | | |
| GO: 0000276 | mitochondrial proton-transporting | — | >0.05 | 3/959 | 1.14E−02 | — | >0.05 |
| | ATP synthase complex, coupling | | | | | | |
| | factor F(o) | | | | | | |
| GO: 0000281 | mitotic cytokinesis | 11/810 | 1.13E−03 | 12/941 | 1.15E−03 | — | >0.05 |
| GO: 0000302 | response to reactive oxygen | 20/810 | 6.79E−04 | 22/941 | 7.57E−04 | — | >0.05 |
| | species | | | | | | |
| GO: 0000375 | RNA splicing, via transesterification | 37/810 | 1.23E−07 | 38/941 | 1.73E−06 | — | >0.05 |
| | reactions | | | | | | |
| GO: 0000377 | RNA splicing, via transesterification | 37/810 | 8.91E−08 | 38/941 | 1.28E−06 | — | >0.05 |
| | reactions with bulged adenosine as | | | | | | |
| | nucleophile | | | | | | |
| GO: 0000380 | alternative mRNA splicing, via | 12/810 | 6.73E−05 | 12/941 | 2.74E−04 | 16/1792 | 1.32E−03 |
| | spliceosome | | | | | | |
| GO: 0000381 | regulation of alternative mRNA | 10/810 | 1.49E−04 | 10/941 | 4.95E−04 | 14/1792 | 7.67E−04 |
| | splicing, via spliceosome | | | | | | |
| GO: 0000398 | mRNA splicing, via spliceosome | 37/810 | 8.91E−08 | 38/941 | 1.28E−06 | — | >0.05 |
| GO: 0000421 | autophagosome membrane | 7/820 | 5.68E−03 | 7/959 | 1.29E−02 | — | >0.05 |
| GO: 0000422 | autophagy of mitochondrion | 12/810 | 5.24E−04 | 12/941 | 1.91E−03 | — | >0.05 |
| GO: 0000502 | proteasome complex | 12/820 | 7.18E−06 | 12/959 | 3.42E−05 | — | >0.05 |
| GO: 0000723 | telomere maintenance | 15/810 | 3.83E−03 | 16/941 | 6.41E−03 | — | >0.05 |
| GO: 0000768 | syncytium formation by plasma | — | >0.05 | 9/941 | 4.14E−03 | — | >0.05 |
| | membrane fusion | | | | | | |
| GO: 0000775 | chromosome, centromeric region | 27/820 | 8.32E−06 | 28/959 | 4.92E−05 | — | >0.05 |
| GO: 0000779 | condensed chromosome, | 14/820 | 1.44E−02 | — | >0.05 | — | >0.05 |
| | centromeric region | | | | | | |
| GO: 0000781 | chromosome, telomeric region | 22/820 | 2.93E−06 | 23/959 | 1.09E−05 | — | >0.05 |
| GO: 0000786 | nucleosome | 26/820 | 1.50E−11 | 27/959 | 8.50E−11 | 27/1866 | 5.60E−05 |
| GO: 0000791 | euchromatin | 10/820 | 1.55E−04 | 11/959 | 1.20E−04 | — | >0.05 |
| GO: 0000792 | heterochromatin | 12/820 | 1.60E−04 | 12/959 | 6.60E−04 | — | >0.05 |
| GO: 0000815 | ESCRT III complex | 4/820 | 8.56E−04 | 4/959 | 1.54E−03 | — | >0.05 |
| GO: 0000910 | cytokinesis | 17/810 | 3.47E−03 | 19/941 | 3.10E−03 | — | >0.05 |
| GO: 0000932 | P-body | 11/820 | 3.06E−03 | 11/959 | 9.67E−03 | — | >0.05 |
| GO: 0000956 | nuclear-transcribed mRNA | 13/810 | 3.28E−03 | 14/941 | 4.51E−03 | — | >0.05 |
| | catabolic process | | | | | | |
| GO: 0001046 | core promoter sequencE-specific | 8/812 | 5.61E−04 | 8/949 | 1.54E−03 | — | >0.05 |
| | DNA binding | | | | | | |
| GO: 0001101 | response to acid chemical | 15/810 | 9.16E−04 | 16/941 | 1.51E−03 | — | >0.05 |
| GO: 0001216 | DNA-binding transcription activator | — | >0.05 | 41/949 | 9.60E−04 | 78/1815 | 5.12E−06 |
| | activity | | | | | | |
| GO: 0001221 | transcription coregulator binding | 15/812 | 1.26E−04 | 16/949 | 2.11E−04 | — | >0.05 |
| GO: 0001222 | transcription corepressor binding | 7/812 | 3.61E−03 | — | >0.05 | — | >0.05 |
| GO: 0001228 | DNA-binding transcription activator | — | >0.05 | 41/949 | 8.14E−04 | 77/1815 | 6.91E−06 |
| | activity, RNA polymerase II-specific | | | | | | |
| GO: 0001503 | ossification | 30/810 | 7.88E−03 | 36/941 | 2.22E−03 | 68/1792 | 3.05E−05 |
| GO: 0001558 | regulation of cell growth | 31/810 | 3.32E−03 | 34/941 | 5.25E−03 | — | >0.05 |
| GO: 0001649 | osteoblast differentiation | 23/810 | 6.56E−04 | 27/941 | 1.87E−04 | 41/1792 | 5.52E−04 |
| GO: 0001654 | eye development | — | >0.05 | — | >0.05 | 62/1792 | 5.53E−05 |
| GO: 0001655 | urogenital system development | — | >0.05 | — | >0.05 | 17/1792 | 1.48E−04 |
| GO: 0001656 | metanephros development | — | >0.05 | — | >0.05 | 20/1792 | 4.76E−04 |
| GO: 0001666 | response to hypoxia | 27/810 | 2.06E−04 | 29/941 | 4.41E−04 | 46/1792 | 6.20E−04 |
| GO: 0001667 | ameboidal-type cell migration | 43/810 | 1.98E−05 | 49/941 | 8.57E−06 | — | >0.05 |
| GO: 0001701 | in utero embryonic development | 30/810 | 3.02E−03 | 36/941 | 6.73E−04 | — | >0.05 |
| GO: 0001704 | formation of primary germ layer | — | >0.05 | | >0.05 | 24/1792 | 1.18E−03 |
| GO: 0001708 | cell fate specification | — | >0.05 | — | >0.05 | 28/1792 | 1.31E−06 |
| GO: 0001725 | stress fiber | 8/820 | 8.96E−03 | — | >0.05 | — | >0.05 |
| GO: 0001726 | ruffle | 22/820 | 9.12E−06 | 22/959 | 9.65E−05 | 32/1866 | 5.68E−04 |
| GO: 0001732 | formation of cytoplasmic translation | 8/810 | 1.30E−07 | 8/941 | 4.12E−07 | 9/1792 | 4.77E−06 |
| | initiation complex | | | | | | |
| GO: 0001772 | immunological synapse | 8/820 | 4.10E−04 | 8/959 | 1.15E−03 | — | >0.05 |
| GO: 0001776 | leukocyte homeostasis | 14/810 | 1.53E−04 | 15/941 | 2.12E−04 | — | >0.05 |
| GO: 0001778 | plasma membrane repair | 9/810 | 2.74E−06 | 9/941 | 9.29E−06 | 10/1792 | 2.69E−04 |
| GO: 0001784 | phosphotyrosine residue binding | 8/812 | 1.04E−03 | 8/949 | 2.78E−03 | — | >0.05 |
| GO: 0001818 | negative regulation of cytokine | 28/810 | 3.86E−03 | — | >0.05 | — | >0.05 |
| | production | | | | | | |
| GO: 0001819 | positive regulation of cytokine | 41/810 | 5.45E−05 | 42/941 | 6.47E−04 | — | >0.05 |
| | production | | | | | | |
| GO: 0001825 | blastocyst formation | — | >0.05 | 7/941 | 7.50E−03 | — | >0.05 |
| GO: 0001829 | trophectodermal cell differentiation | 4/810 | 8.58E−03 | — | >0.05 | — | >0.05 |
| GO: 0001890 | placenta development | 18/810 | 1.32E−04 | 21/941 | 3.28E−05 | 28/1792 | 7.34E−04 |
| GO: 0001891 | phagocytic cup | 7/820 | 1.36E−04 | 8/959 | 4.78E−05 | 9/1866 | 9.45E−04 |
| GO: 0001892 | embryonic placenta development | 11/810 | 1.82E−03 | 12/941 | 1.91E−03 | 18/1792 | 2.13E−03 |
| GO: 0001893 | maternal placenta development | 6/810 | 3.93E−03 | — | >0.05 | — | >0.05 |
| GO: 0001906 | cell killing | 23/810 | 1.74E−05 | 23/941 | 1.70E−04 | — | >0.05 |
| GO: 0001909 | leukocyte mediated cytotoxicity | 15/810 | 1.07E−03 | 15/941 | 4.48E−03 | — | >0.05 |
| GO: 0001933 | negative regulation of protein | 39/810 | 4.96E−08 | 40/941 | 8.59E−07 | 62/1792 | 9.74E−07 |
| | phosphorylation | | | | | | |
| GO: 0001959 | regulation of cytokinE-mediated | 19/810 | 1.27E−04 | 19/941 | 8.15E−04 | — | >0.05 |
| | signaling pathway | | | | | | |
| GO: 0001961 | positive regulation of cytokine- | 8/810 | 4.19E−03 | — | >0.05 | — | >0.05 |
| | mediated signaling pathway | | | | | | |
| GO: 0002020 | protease binding | 16/812 | 4.21E−04 | 17/949 | 8.13E−04 | — | >0.05 |
| GO: 0002090 | regulation of receptor internalization | 9/810 | 1.69E−03 | 11/941 | 3.22E−04 | 14/1792 | 2.59E−03 |
| GO: 0002091 | negative regulation of receptor | 4/810 | 5.65E−03 | — | >0.05 | — | >0.05 |
| | internalization | | | | | | |
| GO: 0002181 | cytoplasmic translation | 63/810 | 7.00E−44 | 64/941 | 4.69E−41 | 69/1792 | 6.32E−29 |
| GO: 0002183 | cytoplasmic translational initiation | 11/810 | 3.44E−07 | 11/941 | 1.51E−06 | 12/1792 | 1.31E−04 |
| GO: 0002218 | activation of innate immune | 32/810 | 9.41E−09 | 33/941 | 9.29E−08 | 39/1792 | 4.88E−04 |
| | response | | | | | | |
| GO: 0002221 | pattern recognition receptor | 23/810 | 8.74E−06 | 24/941 | 3.20E−05 | — | >0.05 |
| | signaling pathway | | | | | | |
| GO: 0002227 | innate immune response in mucosa | 7/810 | 1.30E−04 | 7/941 | 3.27E−04 | — | >0.05 |
| GO: 0002228 | natural killer cell mediated immunity | 10/810 | 2.42E−03 | 10/941 | 6.95E−03 | — | >0.05 |
| GO: 0002237 | response to molecule of bacterial | 29/810 | 1.69E−03 | — | >0.05 | — | >0.05 |
| | origin | | | | | | |
| GO: 0002251 | organ or tissue specific immune | 7/810 | 2.22E−03 | 7/941 | 5.09E−03 | — | >0.05 |
| | response | | | | | | |
| GO: 0002253 | activation of immune response | 59/810 | 2.70E−12 | 61/941 | 1.40E−10 | 70/1792 | 8.32E−04 |
| GO: 0002260 | lymphocyte homeostasis | 10/810 | 1.07E−03 | 11/941 | 9.21E−04 | — | >0.05 |
| GO: 0002262 | myeloid cell homeostasis | 38/810 | 8.10E−17 | 39/941 | 1.83E−15 | 40/1792 | 1.26E−07 |
| GO: 0002263 | cell activation involved in immune | 31/810 | 8.95E−06 | 31/941 | 1.50E−04 | — | >0.05 |
| | response | | | | | | |
| GO: 0002274 | myeloid leukocyte activation | 26/810 | 2.15E−05 | 27/941 | 1.01E−04 | 38/1792 | 1.79E−03 |
| GO: 0002275 | myeloid cell activation involved in | 13/810 | 3.31E−04 | 13/941 | 1.33E−03 | — | >0.05 |
| | immune response | | | | | | |
| GO: 0002279 | mast cell activation involved in | 8/810 | 2.69E−03 | 8/941 | 6.62E−03 | — | >0.05 |
| | immune response | | | | | | |
| GO: 0002283 | neutrophil activation involved in | 4/810 | 8.58E−03 | — | >0.05 | — | >0.05 |
| | immune response | | | | | | |
| GO: 0002285 | lymphocyte activation involved in | 19/810 | 2.10E−03 | — | >0.05 | — | >0.05 |
| | immune response | | | | | | |
| GO: 0002286 | T cell activation involved in immune | 12/810 | 6.32E−03 | — | >0.05 | — | >0.05 |
| | response | | | | | | |
| GO: 0002291 | T cell activation via T cell receptor | 3/810 | 8.14E−03 | — | >0.05 | — | >0.05 |
| | contact with antigen bound to MHC | | | | | | |
| | molecule on antigen presenting cell | | | | | | |
| GO: 0002357 | defense response to tumor cell | 4/810 | 1.95E−03 | 4/941 | 3.38E−03 | — | >0.05 |
| GO: 0002366 | leukocyte activation involved in | 30/810 | 1.81E−05 | 30/941 | 2.66E−04 | — | >0.05 |
| | immune response | | | | | | |
| GO: 0002385 | mucosal immune response | 7/810 | 1.21E−03 | 7/941 | 2.84E−03 | — | >0.05 |
| GO: 0002429 | immune response-activating cell | 35/810 | 1.73E−07 | 36/941 | 2.05E−06 | — | >0.05 |
| | surface receptor signaling pathway | | | | | | |
| GO: 0002431 | Fc receptor mediated stimulatory | 8/810 | 5.68E−05 | 9/941 | 2.26E−05 | 9/1792 | 2.77E−03 |
| | signaling pathway | | | | | | |
| GO: 0002433 | immune response-regulating cell | 7/810 | 9.99E−05 | 7/941 | 2.53E−04 | — | >0.05 |
| | surface receptor signaling pathway | | | | | | |
| | involved in phagocytosis | | | | | | |
| GO: 0002443 | leukocyte mediated immunity | 32/810 | 7.56E−04 | — | >0.05 | — | >0.05 |
| GO: 0002444 | myeloid leukocyte mediated | 13/810 | 9.37E−04 | — | >0.05 | — | >0.05 |
| | immunity | | | | | | |
| GO: 0002478 | antigen processing and | 6/810 | 7.71E−03 | — | >0.05 | — | >0.05 |
| | presentation of exogenous peptide | | | | | | |
| | antigen | | | | | | |
| GO: 0002495 | antigen processing and | 7/810 | 7.22E−04 | 7/941 | 1.73E−03 | — | >0.05 |
| | presentation of peptide antigen via | | | | | | |
| | MHC class II | | | | | | |
| GO: 0002504 | antigen processing and | 7/810 | 1.02E−03 | 7/941 | 2.43E−03 | — | >0.05 |
| | presentation of peptide or | | | | | | |
| | polysaccharide antigen via MHC | | | | | | |
| | class II | | | | | | |
| GO: 0002507 | tolerance induction | — | >0.05 | — | >0.05 | 9/1792 | 1.69E−03 |
| GO: 0002573 | myeloid leukocyte differentiation | 21/810 | 1.08E−03 | 23/941 | 1.37E−03 | 36/1792 | 2.25E−03 |
| GO: 0002683 | negative regulation of immune | 44/810 | 5.63E−06 | 49/941 | 5.20E−06 | — | >0.05 |
| | system process | | | | | | |
| GO: 0002685 | regulation of leukocyte migration | 20/810 | 2.50E−03 | 22/941 | 3.00E−03 | — | >0.05 |
| GO: 0002687 | positive regulation of leukocyte | 15/810 | 2.16E−03 | — | >0.05 | — | >0.05 |
| | migration | | | | | | |
| GO: 0002690 | positive regulation of leukocyte | 11/810 | 3.34E−03 | — | >0.05 | — | >0.05 |
| | chemotaxis | | | | | | |
| GO: 0002695 | negative regulation of leukocyte | 23/810 | 3.08E−05 | 24/941 | 1.11E−04 | — | >0.05 |
| | activation | | | | | | |
| GO: 0002696 | positive regulation of leukocyte | 36/810 | 1.14E−05 | 38/941 | 5.30E−05 | — | >0.05 |
| | activation | | | | | | |
| GO: 0002697 | regulation of immune effector | 32/810 | 4.09E−04 | — | >0.05 | — | >0.05 |
| | process | | | | | | |
| GO: 0002699 | positive regulation of immune | 23/810 | 1.60E−03 | — | >0.05 | — | >0.05 |
| | effector process | | | | | | |
| GO: 0002753 | cytosolic pattern recognition | 16/810 | 2.81E−06 | 17/941 | 4.38E−06 | 19/1792 | 1.44E−03 |
| | receptor signaling pathway | | | | | | |
| GO: 0002757 | immune response-activating | 54/810 | 1.52E−12 | 56/941 | 4.98E−11 | 64/1792 | 2.17E−04 |
| | signaling pathway | | | | | | |
| GO: 0002758 | innate immune response-activating | 26/810 | 1.55E−06 | 27/941 | 7.78E−06 | — | >0.05 |
| | signaling pathway | | | | | | |
| GO: 0002764 | immune response-regulating | 56/810 | 1.54E−12 | 58/941 | 6.12E−11 | 67/1792 | 2.37E−04 |
| | signaling pathway | | | | | | |
| GO: 0002768 | immune response-regulating cell | 37/810 | 1.55E−07 | 38/941 | 2.17E−06 | — | >0.05 |
| | surface receptor signaling pathway | | | | | | |
| GO: 0002790 | peptide secretion | | >0.05 | 23/941 | 3.31E−03 | 38/1792 | 2.25E−03 |
| GO: 0002831 | regulation of response to biotic | 53/810 | 1.75E−10 | 54/941 | 1.22E−08 | 67/1792 | 6.01E−04 |
| | stimulus | | | | | | |
| GO: 0002832 | negative regulation of response to | 17/810 | 3.52E−05 | 17/941 | 2.18E−04 | — | >0.05 |
| | biotic stimulus | | | | | | |
| GO: 0002833 | positive regulation of response to | 37/810 | 1.33E−07 | 38/941 | 1.87E−06 | 48/1792 | 2.42E−03 |
| | biotic stimulus | | | | | | |
| GO: 0002861 | regulation of inflammatory response | 7/810 | 4.27E−03 | 8/941 | 2.45E−03 | — | >0.05 |
| | to antigenic stimulus | | | | | | |
| GO: 0002886 | regulation of myeloid leukocyte | 9/810 | 1.05E−03 | 9/941 | 2.94E−03 | — | >0.05 |
| | mediated immunity | | | | | | |
| GO: 0003002 | regionalization | — | >0.05 | — | >0.05 | 69/1792 | 7.29E−06 |
| GO: 0003712 | transcription coregulator activity | 39/812 | 5.64E−04 | 41/949 | 3.02E−03 | — | >0.05 |
| GO: 0003713 | transcription coactivator activity | 25/812 | 8.40E−04 | 27/949 | 1.67E−03 | — | >0.05 |
| GO: 0003727 | single-stranded RNA binding | 11/812 | 1.70E−03 | — | >0.05 | — | >0.05 |
| GO: 0003735 | structural constituent of ribosome | 51/812 | 1.89E−28 | 51/949 | 2.68E−25 | 53/1815 | 1.72E−14 |
| GO: 0003743 | translation initiation factor activity | 14/812 | 3.21E−08 | 14/949 | 2.21E−07 | 15/1815 | 9.31E−05 |
| GO: 0003779 | actin binding | 52/812 | 8.89E−11 | 53/949 | 7.35E−09 | 75/1815 | 1.55E−06 |
| GO: 0003924 | GTPase activity | 37/812 | 2.22E−07 | 39/949 | 1.38E−06 | 51/1815 | 9.23E−04 |
| GO: 0003925 | G protein activity | 9/812 | 8.14E−05 | 9/949 | 2.64E−04 | — | >0.05 |
| GO: 0004674 | protein serine/threonine kinase | 33/812 | 1.29E−03 | 39/949 | 3.83E−04 | 65/1815 | 2.49E−04 |
| | activity | | | | | | |
| GO: 0004713 | protein tyrosine kinase activity | 14/812 | 3.36E−03 | 15/949 | 5.40E−03 | — | >0.05 |
| GO: 0004715 | non-membrane spanning protein | 9/812 | 9.89E−05 | 9/949 | 3.19E−04 | — | >0.05 |
| | tyrosine kinase activity | | | | | | |
| GO: 0004722 | protein serine/threonine | — | >0.05 | 12/949 | 3.59E−03 | — | >0.05 |
| | phosphatase activity | | | | | | |
| GO: 0004857 | enzyme inhibitor activity | 37/812 | 1.46E−06 | 39/949 | 8.81E−06 | — | >0.05 |
| GO: 0004860 | protein kinase inhibitor activity | 10/812 | 7.63E−06 | 10/949 | 2.96E−05 | 13/1815 | 8.71E−05 |
| GO: 0005080 | protein kinase C binding | 9/812 | 2.83E−04 | 10/949 | 1.87E−04 | — | >0.05 |
| GO: 0005092 | GDP-dissociation inhibitor activity | 4/812 | 4.71E−03 | — | >0.05 | — | >0.05 |
| GO: 0005200 | structural constituent of | 15/812 | 1.26E−04 | 14/949 | 1.94E−03 | — | >0.05 |
| | cytoskeleton | | | | | | |
| GO: 0005216 | monoatomic ion channel activity | — | >0.05 | — | >0.05 | 64/1815 | 6.63E−04 |
| GO: 0005249 | voltage-gated potassium channel | — | >0.05 | — | >0.05 | 19/1815 | 8.33E−04 |
| | activity | | | | | | |
| GO: 0005261 | monoatomic cation channel activity | — | >0.05 | — | >0.05 | 55/1815 | 4.22E−05 |
| GO: 0005525 | GTP binding | 41/812 | 1.37E−07 | 43/949 | 1.25E−06 | — | >0.05 |
| GO: 0005544 | calcium-dependent phospholipid | — | >0.05 | — | >0.05 | 13/1815 | 1.47E−03 |
| | binding | | | | | | |
| GO: 0005546 | phosphatidylinositol-4,5- | 10/812 | 3.55E−03 | 11/949 | 3.54E−03 | — | >0.05 |
| | bisphosphate binding | | | | | | |
| GO: 0005635 | nuclear envelope | 40/820 | 7.16E−05 | 40/959 | 1.62E−03 | — | >0.05 |
| GO: 0005641 | nuclear envelope lumen | — | >0.05 | 3/959 | 1.14E−02 | — | >0.05 |
| GO: 0005643 | nuclear pore | 12/820 | 8.64E−04 | — | >0.05 | — | >0.05 |
| GO: 0005681 | spliceosomal complex | 27/820 | 7.10E−08 | 28/959 | 4.67E−07 | 32/1866 | 2.23E−03 |
| GO: 0005682 | U5 snRNP | — | >0.05 | 5/959 | 7.09E−03 | — | >0.05 |
| GO: 0005684 | U2-type spliceosomal complex | 9/820 | 1.56E−02 | 10/959 | 1.53E−02 | — | >0.05 |
| GO: 0005686 | U2 snRNP | — | >0.05 | 5/959 | 1.16E−02 | — | >0.05 |
| GO: 0005741 | mitochondrial outer membrane | 17/820 | 7.71E−03 | 18/959 | 1.60E−02 | — | >0.05 |
| GO: 0005743 | mitochondrial inner membrane | 31/820 | 1.49E−02 | 35/959 | 1.64E−02 | — | >0.05 |
| GO: 0005746 | mitochondrial respirasome | 9/820 | 8.83E−03 | 10/959 | 8.24E−03 | — | >0.05 |
| GO: 0005750 | mitochondrial respiratory chain | 4/820 | 1.24E−03 | 4/959 | 2.22E−03 | — | >0.05 |
| | complex III | | | | | | |
| GO: 0005753 | mitochondrial proton-transporting | 6/820 | 4.92E−05 | 7/959 | 9.49E−06 | 7/1866 | 6.54E−04 |
| | ATP synthase complex | | | | | | |
| GO: 0005765 | lysosomal membrane | 44/820 | 2.30E−08 | 45/959 | 7.27E−07 | 60/1866 | 9.70E−04 |
| GO: 0005766 | primary lysosome | 24/820 | 4.37E−08 | 25/959 | 2.04E−07 | 29/1866 | 4.27E−04 |
| GO: 0005769 | early endosome | 39/820 | 4.56E−06 | 40/959 | 6.91E−05 | — | >0.05 |
| GO: 0005770 | late endosome | 30/820 | 9.79E−06 | 29/959 | 3.86E−04 | — | >0.05 |
| GO: 0005771 | multivesicular body | 9/820 | 2.10E−03 | 9/959 | 5.90E−03 | — | >0.05 |
| GO: 0005774 | vacuolar membrane | 46/820 | 6.65E−08 | 49/959 | 4.09E−07 | 65/1866 | 1.14E−03 |
| GO: 0005775 | vacuolar lumen | 24/820 | 4.49E−07 | 26/959 | 6.11E−07 | 34/1866 | 6.64E−05 |
| GO: 0005776 | autophagosome | 11/820 | 1.15E−02 | 12/959 | 1.40E−02 | — | >0.05 |
| GO: 0005786 | signal recognition particle, | 5/820 | 5.23E−05 | 5/959 | 1.10E−04 | 5/1866 | 2.41E−03 |
| | endoplasmic reticulum targeting | | | | | | |
| GO: 0005791 | rough endoplasmic reticulum | 11/820 | 7.08E−04 | 13/959 | 2.01E−04 | 19/1866 | 2.63E−04 |
| GO: 0005798 | Golgi-associated vesicle | 10/820 | 5.41E−03 | 12/959 | 1.87E−03 | — | >0.05 |
| GO: 0005819 | spindle | 36/820 | 5.52E−05 | 38/959 | 2.81E−04 | — | >0.05 |
| GO: 0005828 | kinetochore microtubule | 5/820 | 1.27E−03 | 5/959 | 2.54E−03 | — | >0.05 |
| GO: 0005834 | heterotrimeric G-protein complex | 6/820 | 5.25E−04 | 6/959 | 1.19E−03 | 7/1866 | 8.09E−03 |
| GO: 0005840 | ribosome | 55/820 | 5.78E−27 | 55/959 | 1.20E−23 | 57/1866 | 4.66E−12 |
| GO: 0005844 | polysome | 21/820 | 2.73E−13 | 21/959 | 5.45E−12 | 24/1866 | 6.77E−09 |
| GO: 0005852 | eukaryotic translation initiation | 7/820 | 1.20E−06 | 7/959 | 3.43E−06 | 8/1866 | 2.70E−05 |
| | factor 3 complex | | | | | | |
| GO: 0005874 | microtubule | 33/820 | 1.80E−03 | 35/959 | 6.30E−03 | — | >0.05 |
| GO: 0005876 | spindle microtubule | 10/820 | 2.10E−03 | 10/959 | 6.37E−03 | — | >0.05 |
| GO: 0005884 | actin filament | 14/820 | 1.49E−04 | 16/959 | 6.32E−05 | 20/1866 | 2.51E−03 |
| GO: 0005905 | clathrin-coated pit | 12/820 | 5.67E−05 | 13/959 | 5.89E−05 | 18/1866 | 1.65E−04 |
| GO: 0005912 | adherens junction | 16/820 | 5.62E−03 | 17/959 | 1.09E−02 | — | >0.05 |
| GO: 0005925 | focal adhesion | 87/820 | 1.41E−35 | 89/959 | 6.32E−32 | 101/1866 | 5.15E−18 |
| GO: 0005938 | cell cortex | 40/820 | 2.03E−10 | 42/959 | 1.80E−09 | 55/1866 | 3.60E−06 |
| GO: 0005940 | septin ring | — | >0.05 | — | >0.05 | 5/1866 | 8.16E−03 |
| GO: 0006089 | lactate metabolic process | 5/810 | 6.56E−04 | 5/941 | 1.29E−03 | — | >0.05 |
| GO: 0006090 | pyruvate metabolic process | 16/810 | 3.21E−05 | 16/941 | 1.86E−04 | — | >0.05 |
| GO: 0006091 | generation of precursor metabolites | 58/810 | 9.96E−12 | 64/941 | 7.17E−12 | 81/1792 | 2.10E−06 |
| | and energy | | | | | | |
| GO: 0006096 | glycolytic process | 15/810 | 4.13E−06 | 15/941 | 2.46E−05 | 19/1792 | 4.52E−04 |
| GO: 0006098 | pentose-phosphate shunt | 6/810 | 1.20E−04 | 6/941 | 2.72E−04 | — | >0.05 |
| GO: 0006109 | regulation of carbohydrate | 19/810 | 4.81E−04 | 21/941 | 4.66E−04 | 32/1792 | 8.18E−04 |
| | metabolic process | | | | | | |
| GO: 0006110 | regulation of glycolytic process | 7/810 | 4.82E−03 | — | >0.05 | — | >0.05 |
| GO: 0006119 | oxidative phosphorylation | 21/810 | 4.17E−07 | 22/941 | 1.22E−06 | — | >0.05 |
| GO: 0006122 | mitochondrial electron transport, | 4/810 | 1.95E−03 | 4/941 | 3.38E−03 | — | >0.05 |
| | ubiquinol to cytochrome c | | | | | | |
| GO: 0006140 | regulation of nucleotide metabolic | 14/810 | 4.30E−05 | 15/941 | 5.62E−05 | — | >0.05 |
| | process | | | | | | |
| GO: 0006164 | purine nucleotide biosynthetic | 20/810 | 7.04E−03 | 23/941 | 4.47E−03 | — | >0.05 |
| | process | | | | | | |
| GO: 0006165 | nucleoside diphosphate | 15/810 | 3.99E−05 | 15/941 | 2.12E−04 | 20/1792 | 1.85E−03 |
| | phosphorylation | | | | | | |
| GO: 0006278 | RNA-templated DNA biosynthetic | 10/810 | 9.52E−04 | 10/941 | 2.89E−03 | — | >0.05 |
| | process | | | | | | |
| GO: 0006334 | nucleosome assembly | 24/810 | 1.97E−10 | 26/941 | 1.38E−10 | 28/1792 | 4.91E−06 |
| GO: 0006338 | chromatin remodeling | 44/810 | 2.25E−08 | 48/941 | 3.48E−08 | — | >0.05 |
| GO: 0006364 | rRNA processing | 19/810 | 3.21E−03 | — | >0.05 | — | >0.05 |
| GO: 0006397 | mRNA processing | 47/810 | 6.77E−07 | 49/941 | 8.11E−06 | — | >0.05 |
| GO: 0006401 | RNA catabolic process | 35/810 | 5.58E−07 | 38/941 | 9.36E−07 | — | >0.05 |
| GO: 0006402 | mRNA catabolic process | 31/810 | 7.62E−07 | 34/941 | 7.92E−07 | — | >0.05 |
| GO: 0006403 | RNA localization | 19/810 | 1.11E−03 | — | >0.05 | — | >0.05 |
| GO: 0006413 | translational initiation | 23/810 | 3.20E−09 | 23/941 | 5.20E−08 | 25/1792 | 2.66E−04 |
| GO: 0006446 | regulation of translational initiation | 17/810 | 8.42E−08 | 17/941 | 7.00E−07 | 19/1792 | 2.78E−04 |
| GO: 0006457 | protein folding | 18/810 | 8.89E−03 | — | >0.05 | — | >0.05 |
| GO: 0006469 | negative regulation of protein | 31/810 | 6.83E−09 | 31/941 | 2.04E−07 | 43/1792 | 4.51E−06 |
| | kinase activity | | | | | | |
| GO: 0006470 | protein dephosphorylation | 24/810 | 2.71E−04 | 27/941 | 1.87E−04 | — | >0.05 |
| GO: 0006479 | protein methylation | 16/810 | 5.86E−03 | — | >0.05 | — | >0.05 |
| GO: 0006605 | protein targeting | 34/810 | 3.61E−06 | 36/941 | 1.44E−05 | — | >0.05 |
| GO: 0006606 | protein import into nucleus | 17/810 | 8.88E−04 | 19/941 | 7.02E−04 | — | >0.05 |
| GO: 0006611 | protein export from nucleus | 11/810 | 4.64E−05 | 11/941 | 1.76E−04 | — | >0.05 |
| GO: 0006612 | protein targeting to membrane | 13/810 | 4.92E−03 | 14/941 | 6.84E−03 | — | >0.05 |
| GO: 0006613 | cotranslational protein targeting to | 5/810 | 5.94E−03 | — | >0.05 | — | >0.05 |
| | membrane | | | | | | |
| GO: 0006614 | SRP-dependent cotranslational | 5/810 | 2.32E−03 | 5/941 | 4.44E−03 | — | >0.05 |
| | protein targeting to membrane | | | | | | |
| GO: 0006626 | protein targeting to mitochondrion | 12/810 | 2.09E−03 | 12/941 | 6.93E−03 | — | >0.05 |
| GO: 0006739 | NADP metabolic process | 7/810 | 2.55E−03 | 7/941 | 5.82E−03 | — | >0.05 |
| GO: 0006740 | NADPH regeneration | 6/810 | 2.94E−04 | 6/941 | 6.56E−04 | — | >0.05 |
| GO: 0006754 | ATP biosynthetic process | 14/810 | 5.50E−05 | 15/941 | 7.28E−05 | — | >0.05 |
| GO: 0006757 | ATP generation from ADP | 15/810 | 4.80E−06 | 15/941 | 2.83E−05 | 19/1792 | 5.28E−04 |
| GO: 0006801 | superoxide metabolic process | 12/810 | 7.73E−05 | 13/941 | 7.65E−05 | 16/1792 | 1.54E−03 |
| GO: 0006839 | mitochondrial transport | 17/810 | 4.08E−03 | — | >0.05 | — | >0.05 |
| GO: 0006887 | exocytosis | 30/810 | 4.18E−04 | 34/941 | 2.73E−04 | 58/1792 | 4.18E−05 |
| GO: 0006890 | retrograde vesicle-mediated | 7/810 | 7.52E−03 | — | >0.05 | — | >0.05 |
| | transport, Golgi to endoplasmic | | | | | | |
| | reticulum | | | | | | |
| GO: 0006898 | receptor-mediated endocytosis | 24/810 | 4.09E−04 | 26/941 | 6.72E−04 | — | >0.05 |
| GO: 0006900 | vesicle budding from membrane | — | >0.05 | 11/941 | 3.02E−03 | — | >0.05 |
| GO: 0006906 | vesicle fusion | 13/810 | 2.46E−03 | — | >0.05 | — | >0.05 |
| GO: 0006909 | phagocytosis | 27/810 | 5.47E−06 | 30/941 | 3.86E−06 | — | >0.05 |
| GO: 0006913 | nucleocytoplasmic transport | 36/810 | 4.81E−07 | 38/941 | 2.33E−06 | — | >0.05 |
| GO: 0006949 | syncytium formation | — | >0.05 | 11/941 | 4.88E−04 | — | >0.05 |
| GO: 0006970 | response to osmotic stress | 11/810 | 1.38E−03 | 11/941 | 4.39E−03 | — | >0.05 |
| GO: 0006979 | response to oxidative stress | 43/810 | 4.78E−07 | 47/941 | 7.76E−07 | — | >0.05 |
| GO: 0006997 | nucleus organization | 14/810 | 4.35E−03 | 15/941 | 6.66E−03 | — | >0.05 |
| GO: 0006998 | nuclear envelope organization | 10/810 | 2.02E−04 | 11/941 | 1.50E−04 | — | >0.05 |
| GO: 0007004 | telomere maintenance via | 10/810 | 8.49E−04 | 10/941 | 2.59E−03 | — | >0.05 |
| | telomerase | | | | | | |
| GO: 0007006 | mitochondrial membrane | 12/810 | 5.92E−03 | — | >0.05 | — | >0.05 |
| | organization | | | | | | |
| GO: 0007009 | plasma membrane organization | 20/810 | 5.77E−05 | 22/941 | 5.42E−05 | 30/1792 | 9.12E−04 |
| GO: 0007015 | actin filament organization | 47/810 | 2.02E−08 | 52/941 | 1.70E−08 | 73/1792 | 4.32E−06 |
| GO: 0007032 | endosome organization | 13/810 | 2.98E−04 | 14/941 | 3.71E−04 | — | >0.05 |
| GO: 0007033 | vacuole organization | 21/810 | 5.65E−04 | 23/941 | 6.96E−04 | — | >0.05 |
| GO: 0007034 | vacuolar transport | 15/810 | 6.08E−03 | — | >0.05 | — | >0.05 |
| GO: 0007040 | lysosome organization | 11/810 | 4.24E−03 | 12/941 | 4.67E−03 | — | >0.05 |
| GO: 0007041 | lysosomal transport | 14/810 | 1.94E−03 | 14/941 | 7.31E−03 | — | >0.05 |
| GO: 0007051 | spindle organization | 21/810 | 1.85E−04 | 23/941 | 2.14E−04 | — | >0.05 |
| GO: 0007052 | mitotic spindle organization | 14/810 | 1.81E−03 | 16/941 | 1.01E−03 | — | >0.05 |
| GO: 0007080 | mitotic metaphase plate | 8/810 | 3.38E−03 | — | >0.05 | — | >0.05 |
| | congression | | | | | | |
| GO: 0007156 | homophilic cell adhesion via plasma | — | >0.05 | — | >0.05 | 31/1792 | 3.82E−04 |
| | membrane adhesion molecules | | | | | | |
| GO: 0007159 | leukocyte cell-cell adhesion | 41/810 | 1.20E−06 | 43/941 | 8.91E−06 | — | >0.05 |
| GO: 0007162 | negative regulation of cell adhesion | 31/810 | 2.23E−05 | 33/941 | 6.74E−05 | — | >0.05 |
| GO: 0007163 | establishment or maintenance of | 20/810 | 2.37E−03 | — | >0.05 | — | >0.05 |
| | cell polarity | | | | | | |
| GO: 0007188 | adenylate cyclase-modulating G | — | >0.05 | — | >0.05 | 37/1792 | 2.25E−03 |
| | protein-coupled receptor signaling | | | | | | |
| | pathway | | | | | | |
| GO: 0007213 | G protein-coupled acetylcholine | — | >0.05 | — | >0.05 | 7/1792 | 2.83E−03 |
| | receptor signaling pathway | | | | | | |
| GO: 0007249 | I-kappaB kinase/NF-kappaB | 32/810 | 2.30E−06 | 32/941 | 4.67E−05 | — | >0.05 |
| | signaling | | | | | | |
| GO: 0007254 | JNK cascade | 15/810 | 8.39E−03 | — | >0.05 | — | >0.05 |
| GO: 0007264 | small GTPase mediated signal | 47/810 | 3.34E−07 | 47/941 | 1.93E−05 | — | >0.05 |
| | transduction | | | | | | |
| GO: 0007265 | Ras protein signal transduction | 32/810 | 3.21E−05 | 31/941 | 1.01E−03 | — | >0.05 |
| GO: 0007389 | pattern specification process | — | >0.05 | — | >0.05 | 79/1792 | 4.35E−07 |
| GO: 0007398 | ectoderm development | — | >0.05 | — | >0.05 | 9/1792 | 7.79E−05 |
| GO: 0007405 | neuroblast proliferation | 9/810 | 4.70E−03 | 11/941 | 1.17E−03 | 19/1792 | 5.26E−05 |
| GO: 0007409 | axonogenesis | — | >0.05 | — | >0.05 | 61/1792 | 1.57E−03 |
| GO: 0007411 | axon guidance | — | >0.05 | — | >0.05 | 38/1792 | 5.68E−04 |
| GO: 0007416 | synapse assembly | — | >0.05 | — | >0.05 | 33/1792 | 1.33E−03 |
| GO: 0007498 | mesoderm development | — | >0.05 | — | >0.05 | 28/1792 | 1.08E−04 |
| GO: 0007517 | muscle organ development | — | >0.05 | — | >0.05 | 51/1792 | 2.13E−03 |
| GO: 0007519 | skeletal muscle tissue development | — | >0.05 | 20/941 | 2.41E−04 | 31/1792 | 1.96E−04 |
| GO: 0007530 | sex determination | — | >0.05 | — | >0.05 | 8/1792 | 7.41E−04 |
| GO: 0008021 | synaptic vesicle | — | >0.05 | 19/959 | 8.72E−03 | 36/1866 | 4.97E−04 |
| GO: 0008064 | regulation of actin polymerization or | 19/810 | 2.84E−05 | 19/941 | 2.05E−04 | — | >0.05 |
| | depolymerization | | | | | | |
| GO: 0008088 | axo-dendritic transport | 9/810 | 7.26E−03 | 10/941 | 6.36E−03 | — | >0.05 |
| GO: 0008135 | translation factor activity, RNA | 18/812 | 2.08E−08 | 18/949 | 2.18E−07 | 20/1815 | 1.54E−04 |
| | binding | | | | | | |
| GO: 0008154 | actin polymerization or | 27/810 | 8.80E−08 | 29/941 | 1.48E−07 | 34/1792 | 4.10E−04 |
| | depolymerization | | | | | | |
| GO: 0008157 | protein phosphatase 1 binding | 6/812 | 3.18E−04 | 6/949 | 7.31E−04 | — | >0.05 |
| GO: 0008213 | protein alkylation | 16/810 | 5.86E−03 | — | >0.05 | — | >0.05 |
| GO: 0008286 | insulin receptor signaling pathway | 13/810 | 4.32E−03 | 14/941 | 5.98E−03 | — | >0.05 |
| GO: 0008287 | protein serine/threonine | 7/820 | 6.33E−03 | 8/959 | 4.01E−03 | — | >0.05 |
| | phosphatase complex | | | | | | |
| GO: 0008303 | caspase complex | 3/820 | 7.43E−03 | 3/959 | 1.14E−02 | — | >0.05 |
| GO: 0008333 | endosome to lysosome transport | 11/810 | 3.36E−04 | 11/941 | 1.17E−03 | 17/1792 | 5.37E−04 |
| GO: 0008344 | adult locomotory behavior | 11/810 | 8.33E−04 | 12/941 | 8.27E−04 | 19/1792 | 2.35E−04 |
| GO: 0008360 | regulation of cell shape | 16/810 | 3.90E−04 | 16/941 | 1.90E−03 | — | >0.05 |
| GO: 0008380 | RNA splicing | 48/810 | 3.19E−08 | 51/941 | 1.94E−07 | — | >0.05 |
| GO: 0008625 | extrinsic apoptotic signaling | 13/810 | 9.64E−05 | 13/941 | 4.18E−04 | — | >0.05 |
| | pathway via death domain receptors | | | | | | |
| GO: 0008630 | intrinsic apoptotic signaling pathway | 13/810 | 5.96E−04 | 13/941 | 2.31E−03 | — | >0.05 |
| | in response to DNA damage | | | | | | |
| GO: 0008631 | intrinsic apoptotic signaling pathway | 10/810 | 2.07E−05 | 10/941 | 7.39E−05 | — | >0.05 |
| | in response to oxidative stress | | | | | | |
| GO: 0008643 | carbohydrate transport | — | >0.05 | — | >0.05 | 27/1792 | 2.12E−03 |
| GO: 0008645 | hexose transmembrane transport | — | >0.05 | — | >0.05 | 22/1792 | 1.98E−03 |
| GO: 0009060 | aerobic respiration | 27/810 | 3.15E−08 | 28/941 | 1.83E−07 | — | >0.05 |
| GO: 0009132 | nucleoside diphosphate metabolic | 17/810 | 8.58E−05 | 17/941 | 5.00E−04 | — | >0.05 |
| | process | | | | | | |
| GO: 0009135 | purine nucleoside diphosphate | 17/810 | 3.76E−06 | 17/941 | 2.65E−05 | 21/1792 | 1.26E−03 |
| | metabolic process | | | | | | |
| GO: 0009141 | nucleoside triphosphate metabolic | 36/810 | 6.60E−10 | 38/941 | 3.04E−09 | 45/1792 | 6.52E−05 |
| | process | | | | | | |
| GO: 0009142 | nucleoside triphosphate | 15/810 | 2.84E−04 | 16/941 | 4.55E−04 | — | >0.05 |
| | biosynthetic process | | | | | | |
| GO: 0009144 | purine nucleoside triphosphate | 36/810 | 7.25E−11 | 38/941 | 3.20E−10 | 45/1792 | 8.90E−06 |
| | metabolic process | | | | | | |
| GO: 0009145 | purine nucleoside triphosphate | 15/810 | 5.63E−05 | 16/941 | 8.56E−05 | — | >0.05 |
| | biosynthetic process | | | | | | |
| GO: 0009150 | purine ribonucleotide metabolic | 44/810 | 2.10E−07 | 47/941 | 8.86E−07 | — | >0.05 |
| | process | | | | | | |
| GO: 0009152 | purine ribonucleotide biosynthetic | 19/810 | 1.77E−03 | 20/941 | 4.18E−03 | — | >0.05 |
| | process | | | | | | |
| GO: 0009179 | purine ribonucleoside diphosphate | 17/810 | 3.76E−06 | 17/941 | 2.65E−05 | 21/1792 | 1.26E−03 |
| | metabolic process | | | | | | |
| GO: 0009185 | ribonucleoside diphosphate | 17/810 | 1.30E−05 | 17/941 | 8.58E−05 | — | >0.05 |
| | metabolic process | | | | | | |
| GO: 0009199 | ribonucleoside triphosphate | 35/810 | 3.71E−10 | 37/941 | 1.50E−09 | 44/1792 | 2.47E−05 |
| | metabolic process | | | | | | |
| GO: 0009201 | ribonucleoside triphosphate | 15/810 | 9.69E−05 | 16/941 | 1.50E−04 | — | >0.05 |
| | biosynthetic process | | | | | | |
| GO: 0009205 | purine ribonucleoside triphosphate | 35/810 | 1.61E−10 | 37/941 | 6.37E−10 | 44/1792 | 1.15E−05 |
| | metabolic process | | | | | | |
| GO: 0009206 | purine ribonucleoside triphosphate | 15/810 | 5.03E−05 | 16/941 | 7.61E−05 | — | >0.05 |
| | biosynthetic process | | | | | | |
| GO: 0009259 | ribonucleotide metabolic process | 44/810 | 6.97E−07 | 47/941 | 2.95E−06 | — | >0.05 |
| GO: 0009260 | ribonucleotide biosynthetic process | 19/810 | 3.74E−03 | — | >0.05 | — | >0.05 |
| GO: 0009299 | mRNA transcription | 8/810 | 1.64E−03 | 8/941 | 4.14E−03 | — | >0.05 |
| GO: 0009615 | response to virus | 46/810 | 3.92E−09 | 47/941 | 1.34E−07 | 58/1792 | 1.80E−03 |
| GO: 0009648 | photoperiodism | 5/810 | 8.12E−03 | — | >0.05 | — | >0.05 |
| GO: 0009649 | entrainment of circadian clock | 5/810 | 8.12E−03 | — | >0.05 | — | >0.05 |
| GO: 0009678 | pyrophosphate hydrolysis-driven | — | >0.05 | 6/949 | 2.32E−03 | — | >0.05 |
| | proton transmembrane transporter | | | | | | |
| | activity | | | | | | |
| GO: 0009895 | negative regulation of catabolic | 39/810 | 3.32E−08 | 43/941 | 2.79E−08 | 56/1792 | 4.40E−05 |
| | process | | | | | | |
| GO: 0009898 | cytoplasmic side of plasma | 23/820 | 2.63E−07 | 26/959 | 7.72E−08 | 35/1866 | 2.62E−06 |
| | membrane | | | | | | |
| GO: 0009931 | calcium-dependent protein | 5/812 | 3.05E−03 | — | >0.05 | — | >0.05 |
| | serine/threonine kinase activity | | | | | | |
| GO: 0009952 | anterior/posterior pattern | — | >0.05 | — | >0.05 | 39/1792 | 5.65E−05 |
| | specification | | | | | | |
| GO: 0010038 | response to metal ion | — | >0.05 | — | >0.05 | 57/1792 | 2.48E−04 |
| GO: 0010155 | regulation of proton transport | 5/810 | 3.49E−03 | 5/941 | 6.59E−03 | — | >0.05 |
| GO: 0010310 | regulation of hydrogen peroxide | 4/810 | 8.58E−03 | — | >0.05 | — | >0.05 |
| | metabolic process | | | | | | |
| GO: 0010324 | membrane invagination | 8/810 | 7.58E−03 | 9/941 | 5.69E−03 | — | >0.05 |
| GO: 0010458 | exit from mitosis | 5/810 | 8.12E−03 | — | >0.05 | — | >0.05 |
| GO: 0010463 | mesenchymal cell proliferation | — | >0.05 | — | >0.05 | 11/1792 | 2.25E−03 |
| GO: 0010469 | regulation of signaling receptor | — | >0.05 | — | >0.05 | 28/1792 | 2.41E−03 |
| | activity | | | | | | |
| GO: 0010494 | cytoplasmic stress granule | 16/820 | 5.26E−07 | 17/959 | 8.38E−07 | 25/1866 | 2.83E−07 |
| GO: 0010506 | regulation of autophagy | 38/810 | 2.46E−07 | 39/941 | 3.62E−06 | — | >0.05 |
| GO: 0010507 | negative regulation of autophagy | 12/810 | 4.24E−04 | 13/941 | 4.68E−04 | — | >0.05 |
| GO: 0010563 | negative regulation of phosphorus | 54/810 | 8.76E−12 | 57/941 | 9.57E−11 | 83/1792 | 2.61E−09 |
| | metabolic process | | | | | | |
| GO: 0010586 | miRNA metabolic process | 15/810 | 2.17E−05 | 16/941 | 3.18E−05 | 23/1792 | 4.53E−05 |
| GO: 0010591 | regulation of lamellipodium | 10/810 | 1.08E−05 | 10/941 | 3.92E−05 | 11/1792 | 1.84E−03 |
| | assembly | | | | | | |
| GO: 0010592 | positive regulation of lamellipodium | 8/810 | 2.61E−05 | 8/941 | 7.57E−05 | 9/1792 | 1.29E−03 |
| | assembly | | | | | | |
| GO: 0010594 | regulation of endothelial cell | 22/810 | 6.32E−04 | 25/941 | 3.73E−04 | — | >0.05 |
| | migration | | | | | | |
| GO: 0010595 | positive regulation of endothelial | 17/810 | 6.44E−05 | 19/941 | 3.89E−05 | — | >0.05 |
| | cell migration | | | | | | |
| GO: 0010631 | epithelial cell migration | 33/810 | 1.10E−04 | 38/941 | 3.97E−05 | — | >0.05 |
| GO: 0010632 | regulation of epithelial cell migration | 28/810 | 1.21E−04 | 32/941 | 5.33E−05 | — | >0.05 |
| GO: 0010634 | positive regulation of epithelial cell | 22/810 | 1.05E−05 | 25/941 | 3.65E−06 | — | >0.05 |
| | migration | | | | | | |
| GO: 0010639 | negative regulation of organelle | 35/810 | 1.08E−05 | 35/941 | 2.20E−04 | — | >0.05 |
| | organization | | | | | | |
| GO: 0010720 | positive regulation of cell | 32/810 | 2.94E−03 | 35/941 | 4.97E−03 | — | >0.05 |
| | development | | | | | | |
| GO: 0010762 | regulation of fibroblast migration | 6/810 | 7.71E−03 | — | >0.05 | — | >0.05 |
| GO: 0010803 | regulation of tumor necrosis factor- | 8/810 | 3.02E−03 | 8/941 | 7.39E−03 | — | >0.05 |
| | mediated signaling pathway | | | | | | |
| GO: 0010821 | regulation of mitochondrion | 17/810 | 3.68E−04 | 17/941 | 1.92E−03 | — | >0.05 |
| | organization | | | | | | |
| GO: 0010823 | negative regulation of | 7/810 | 8.35E−03 | — | >0.05 | — | >0.05 |
| | mitochondrion organization | | | | | | |
| GO: 0010824 | regulation of centrosome | 8/810 | 9.46E−04 | 8/941 | 2.45E−03 | — | >0.05 |
| | duplication | | | | | | |
| GO: 0010833 | telomere maintenance via telomere | 12/810 | 2.15E−04 | 12/941 | 8.27E−04 | — | >0.05 |
| | lengthening | | | | | | |
| GO: 0010857 | calcium-dependent protein kinase | 5/812 | 3.71E−03 | — | >0.05 | — | >0.05 |
| | activity | | | | | | |
| GO: 0010923 | negative regulation of phosphatase | 6/810 | 3.93E−03 | — | >0.05 | — | >0.05 |
| | activity | | | | | | |
| GO: 0010939 | regulation of necrotic cell death | 7/810 | 6.06E−03 | 8/941 | 3.65E−03 | — | >0.05 |
| GO: 0010950 | positive regulation of | 18/810 | 5.69E−04 | 18/941 | 3.02E−03 | — | >0.05 |
| | endopeptidase activity | | | | | | |
| GO: 0010952 | positive regulation of peptidase | 19/810 | 5.51E−04 | 19/941 | 3.10E−03 | — | >0.05 |
| | activity | | | | | | |
| GO: 0012510 | trans-Golgi network transport | 4/820 | 1.11E−02 | 5/959 | 3.20E−03 | — | >0.05 |
| | vesicle membrane | | | | | | |
| GO: 0014002 | astrocyte development | — | >0.05 | 7/941 | 5.82E−03 | — | >0.05 |
| GO: 0014065 | phosphatidylinositol 3-kinase | 14/810 | 4.63E−03 | 16/941 | 2.93E−03 | — | >0.05 |
| | signaling | | | | | | |
| GO: 0014069 | postsynaptic density | 33/820 | 9.12E−06 | 37/959 | 6.61E−06 | 70/1866 | 7.57E−10 |
| GO: 0015078 | proton transmembrane transporter | 14/812 | 2.74E−03 | 19/949 | 7.03E−05 | 26/1815 | 7.22E−04 |
| | activity | | | | | | |
| GO: 0015252 | proton channel activity | 7/812 | 6.23E−05 | 8/949 | 1.89E−05 | 8/1815 | 1.68E−03 |
| GO: 0015267 | channel activity | — | >0.05 | — | >0.05 | 70/1815 | 5.16E−04 |
| GO: 0015629 | actin cytoskeleton | 57/820 | 4.48E−12 | 60/959 | 8.75E−11 | 86/1866 | 2.84E−08 |
| GO: 0015671 | oxygen transport | — | >0.05 | 4/941 | 7.61E−03 | — | >0.05 |
| GO: 0015749 | monosaccharide transmembrane | — | >0.05 | — | >0.05 | 23/1792 | 1.21E−03 |
| | transport | | | | | | |
| GO: 0015833 | peptide transport | — | >0.05 | 23/941 | 7.16E−03 | — | >0.05 |
| GO: 0015934 | large ribosomal subunit | 31/820 | 5.41E−17 | 31/959 | 4.11E−15 | 32/1866 | 2.23E−08 |
| GO: 0015935 | small ribosomal subunit | 21/820 | 2.40E−12 | 21/959 | 4.56E−11 | 22/1866 | 1.18E−06 |
| GO: 0015980 | energy derivation by oxidation of | 35/810 | 8.09E−07 | 39/941 | 5.19E−07 | 49/1792 | 1.08E−03 |
| | organic compounds | | | | | | |
| GO: 0015986 | proton motive forcE−driven ATP | 10/810 | 5.24E−04 | 11/941 | 4.26E−04 | — | >0.05 |
| | synthesis | | | | | 2 | |
| GO: 0016032 | viral process | 54/810 | 1.84E−12 | 57/941 | 1.98E−11 | 64/179 | 2.48E−04 |
| GO: 0016049 | cell growth | 37/810 | 1.15E−03 | 42/941 | 8.60E−04 | — | >0.05 |
| GO: 0016050 | vesicle organization | 30/810 | 8.32E−04 | 35/941 | 2.86E−04 | — | >0.05 |
| GO: 0016052 | carbohydrate catabolic process | 18/810 | 2.73E−04 | 18/941 | 1.55E−03 | — | >0.05 |
| GO: 0016072 | rRNA metabolic process | 25/810 | 1.57E−04 | 25/941 | 1.39E−03 | — | >0.05 |
| GO: 0016197 | endosomal transport | 25/810 | 9.42E−05 | 27/941 | 1.64E−04 | — | >0.05 |
| GO: 0016209 | antioxidant activity | 10/812 | 3.55E−03 | 11/949 | 3.54E−03 | — | >0.05 |
| GO: 0016234 | inclusion body | 11/820 | 2.88E−04 | 11/959 | 1.06E−03 | — | >0.05 |
| GO: 0016235 | aggresome | 8/820 | 1.12E−04 | 8/959 | 3.26E−04 | 10/1866 | 1.75E−03 |
| GO: 0016236 | macroautophagy | 36/810 | 2.43E−07 | 38/941 | 1.18E−06 | — | >0.05 |
| GO: 0016241 | regulation of macroautophagy | 19/810 | 7.05E−05 | 20/941 | 1.72E−04 | — | >0.05 |
| GO: 0016281 | eukaryotic translation initiation | 5/820 | 8.65E−05 | 5/959 | 1.81E−04 | 5/1866 | 3.81E−03 |
| | factor 4F complex | | | | | | |
| GO: 0016282 | eukaryotic 43S preinitiation | 7/820 | 3.37E−06 | 7/959 | 9.49E−06 | 8/1866 | 8.52E−05 |
| | complex | | | | | | |
| GO: 0016311 | dephosphorylation | 28/810 | 1.24E−03 | 31/941 | 1.48E−03 | — | >0.05 |
| GO: 0016339 | calcium-dependent cell-cell | — | >0.05 | — | >0.05 | 13/1792 | 4.11E−04 |
| | adhesion via plasma membrane cell | | | | | | |
| | adhesion molecules | | | | | | |
| GO: 0016363 | nuclear matrix | 14/820 | 1.06E−03 | 16/959 | 5.93E−04 | — | >0.05 |
| GO: 0016469 | proton-transporting two-sector | 9/820 | 1.20E−04 | 12/959 | 1.81E−06 | 12/1866 | 1.16E−03 |
| | ATPase complex | | | | | | |
| GO: 0016471 | vacuolar proton-transporting V-type | — | >0.05 | 5/959 | 7.09E−03 | — | >0.05 |
| | ATPase complex | | | | | | |
| GO: 0016479 | negative regulation of transcription | 3/810 | 8.14E−03 | — | >0.05 | — | >0.05 |
| | by RNA polymerase | | | | | | |
| GO: 0016482 | cytosolic transport | 17/810 | 1.94E−03 | 20/941 | 6.63E−04 | — | >0.05 |
| GO: 0016538 | cyclin-dependent protein | 8/812 | 1.58E−03 | 8/949 | 4.14E−03 | — | >0.05 |
| | serine/threonine kinase regulator | | | | | | |
| | activity | | | | | | |
| GO: 0016540 | protein autoprocessing | — | >0.05 | — | >0.05 | 9/1792 | 9.75E−04 |
| GO: 0016574 | histone ubiquitination | 7/810 | 3.78E−03 | — | >0.05 | — | >0.05 |
| GO: 0016601 | Rac protein signal transduction | 7/810 | 2.55E−03 | — | >0.05 | — | >0.05 |
| GO: 0016605 | PML body | 13/820 | 4.83E−04 | 13/959 | 2.01E−03 | — | >0.05 |
| GO: 0016607 | nuclear speck | 35/820 | 1.06E−04 | 36/959 | 9.49E−04 | — | >0.05 |
| GO: 0016922 | nuclear receptor binding | 18/812 | 4.46E−05 | 19/949 | 1.05E−04 | — | >0.05 |
| GO: 0017018 | myosin phosphatase activity | 10/812 | 2.01E−03 | 12/949 | 5.51E−04 | — | >0.05 |
| GO: 0017053 | transcription repressor complex | — | >0.05 | 9/959 | 1.55E−02 | — | >0.05 |
| GO: 0017124 | SH3 domain binding | — | >0.05 | 15/949 | 1.92E−03 | — | >0.05 |
| GO: 0017157 | regulation of exocytosis | 21/810 | 1.39E−04 | 23/941 | 1.58E−04 | 37/1792 | 5.36E−05 |
| GO: 0018105 | peptidyl-serine phosphorylation | 30/810 | 4.64E−05 | 36/941 | 3.76E−06 | 53/1792 | 3.44E−05 |
| GO: 0018108 | peptidyl-tyrosine phosphorylation | 28/810 | 4.01E−03 | 31/941 | 5.08E−03 | — | >0.05 |
| GO: 0018209 | peptidyl-serine modification | 30/810 | 1.30E−04 | 36/941 | 1.35E−05 | 53/1792 | 1.57E−04 |
| GO: 0018212 | peptidyl-tyrosine modification | 28/810 | 4.31E−03 | 31/941 | 5.49E−03 | — | >0.05 |
| GO: 0018958 | phenol-containing compound | — | >0.05 | — | >0.05 | 22/1792 | 9.68E−04 |
| | metabolic process | | | | | | |
| GO: 0019001 | guanyl nucleotide binding | 41/812 | 6.26E−07 | 43/949 | 5.48E−06 | 59/1815 | 1.38E−03 |
| GO: 0019003 | GDP binding | 12/812 | 8.88E−05 | 12/949 | 3.77E−04 | — | >0.05 |
| GO: 0019058 | viral life cycle | 42/810 | 2.31E−10 | 43/941 | 6.45E−09 | 49/1792 | 9.44E−04 |
| GO: 0019068 | virion assembly | 8/810 | 1.40E−04 | 8/941 | 3.88E−04 | — | >0.05 |
| GO: 0019076 | viral release from host cell | 10/810 | 2.47E−07 | 10/941 | 9.80E−07 | 11/1792 | 4.87E−05 |
| GO: 0019079 | viral genome replication | 20/810 | 8.21E−07 | 21/941 | 2.14E−06 | — | >0.05 |
| GO: 0019081 | viral translation | 5/810 | 8.75E−04 | 5/941 | 1.71E−03 | — | >0.05 |
| GO: 0019207 | kinase regulator activity | 24/812 | 2.68E−05 | 24/949 | 2.93E−04 | — | >0.05 |
| GO: 0019208 | phosphatase regulator activity | — | >0.05 | 14/949 | 1.49E−03 | — | >0.05 |
| GO: 0019210 | kinase inhibitor activity | 11/812 | 2.68E−06 | 11/949 | 1.18E−05 | 14/1815 | 6.11E−05 |
| GO: 0019216 | regulation of lipid metabolic process | — | >0.05 | 29/941 | 5.82E−03 | — | >0.05 |
| GO: 0019221 | cytokine-mediated signaling | 35/810 | 3.60E−03 | — | >0.05 | — | >0.05 |
| | pathway | | | | | | |
| GO: 0019362 | pyridine nucleotide metabolic | 9/810 | 7.89E−03 | 10/941 | 6.95E−03 | — | >0.05 |
| | process | | | | | | |
| GO: 0019646 | aerobic electron transport chain | 11/810 | 1.02E−03 | 11/941 | 3.33E−03 | — | >0.05 |
| GO: 0019693 | ribose phosphate metabolic | 46/810 | 1.91E−07 | 49/941 | 9.70E−07 | — | >0.05 |
| | process | | | | | | |
| GO: 0019730 | antimicrobial humoral response | 13/810 | 3.52E−03 | — | >0.05 | — | >0.05 |
| GO: 0019731 | antibacterial humoral response | 8/810 | 6.28E−03 | — | >0.05 | — | >0.05 |
| GO: 0019843 | rRNA binding | 14/812 | 1.51E−06 | 14/949 | 9.16E−06 | — | >0.05 |
| GO: 0019867 | outer membrane | 18/820 | 1.34E−02 | — | >0.05 | — | >0.05 |
| GO: 0019886 | antigen processing and | 6/810 | 2.07E−03 | 6/941 | 4.37E−03 | — | >0.05 |
| | presentation of exogenous peptide | | | | | | |
| | antigen via MHC class II | | | | | | |
| GO: 0019887 | protein kinase regulator activity | 22/812 | 2.66E−05 | 22/949 | 2.56E−04 | — | >0.05 |
| GO: 0019897 | extrinsic component of plasma | 18/820 | 5.18E−05 | 18/959 | 3.62E−04 | 27/1866 | 7.97E−04 |
| | membrane | | | | | | |
| GO: 0019898 | extrinsic component of membrane | 23/820 | 2.82E−03 | 24/959 | 9.26E−03 | — | >0.05 |
| GO: 0019902 | phosphatase binding | 26/812 | 2.52E−07 | 29/949 | 1.27E−07 | 32/1815 | 1.63E−03 |
| GO: 0019903 | protein phosphatase binding | 22/812 | 2.38E−07 | 23/949 | 8.64E−07 | 26/1815 | 1.27E−03 |
| GO: 0020027 | hemoglobin metabolic process | 7/810 | 6.59E−06 | 7/941 | 1.76E−05 | 7/1792 | 1.01E−03 |
| GO: 0021545 | cranial nerve development | — | >0.05 | — | >0.05 | 15/1792 | 5.04E−04 |
| GO: 0021602 | cranial nerve morphogenesis | — | >0.05 | — | >0.05 | 10/1792 | 3.69E−04 |
| GO: 0021675 | nerve development | — | >0.05 | — | >0.05 | 18/1792 | 1.87E−03 |
| GO: 0021700 | developmental maturation | — | >0.05 | — | >0.05 | 49/1792 | 8.20E−04 |
| GO: 0021761 | limbic system development | — | >0.05 | 13/941 | 6.02E−03 | — | >0.05 |
| GO: 0021846 | cell proliferation in forebrain | — | >0.05 | 5/941 | 5.44E−03 | 8/1792 | 1.04E−03 |
| GO: 0021871 | forebrain regionalization | — | >0.05 | — | >0.05 | 8/1792 | 1.43E−03 |
| GO: 0022407 | regulation of cell-cell adhesion | 44/810 | 6.59E−06 | 48/941 | 1.32E−05 | 67/1792 | 2.90E−03 |
| GO: 0022408 | negative regulation of cell-cell | 22/810 | 1.10E−04 | 23/941 | 3.55E−04 | 33/1792 | 2.83E−03 |
| | adhesion | | | | | | |
| GO: 0022409 | positive regulation of cell-cell | 31/810 | 3.64E−05 | 34/941 | 4.80E−05 | — | >0.05 |
| | adhesion | | | | | | |
| GO: 0022411 | cellular component disassembly | 52/810 | 2.68E−09 | 53/941 | 1.46E−07 | — | >0.05 |
| GO: 0022604 | regulation of cell morphogenesis | 21/810 | 2.76E−03 | — | >0.05 | — | >0.05 |
| GO: 0022613 | ribonucleoprotein complex | 50/810 | 6.55E−09 | 50/941 | 6.89E−07 | — | >0.05 |
| | biogenesis | | | | | | |
| GO: 0022618 | ribonucleoprotein complex | 32/810 | 4.90E−09 | 32/941 | 1.61E−07 | 37/1792 | 1.11E−03 |
| | assembly | | | | | | |
| GO: 0022624 | proteasome accessory complex | 5/820 | 2.48E−03 | 5/959 | 4.88E−03 | — | >0.05 |
| GO: 0022625 | cytosolic large ribosomal subunit | 31/820 | 1.77E−27 | 31/959 | 2.03E−25 | 32/1866 | 5.55E−18 |
| GO: 0022626 | cytosolic ribosome | 50/820 | 1.05E−41 | 50/959 | 2.27E−38 | 52/1866 | 1.02E−26 |
| GO: 0022627 | cytosolic small ribosomal subunit | 20/820 | 3.74E−17 | 20/959 | 7.63E−16 | 21/1866 | 1.95E−11 |
| GO: 0022803 | passive transmembrane transporter | — | >0.05 | — | >0.05 | 71/1815 | 3.33E−04 |
| | activity | | | | | | |
| GO: 0022900 | electron transport chain | 17/810 | 9.51E−04 | 19/941 | 7.57E−04 | — | >0.05 |
| GO: 0022904 | respiratory electron transport chain | 14/810 | 3.75E−04 | 14/941 | 1.61E−03 | — | >0.05 |
| GO: 0023023 | MHC protein complex binding | 7/812 | 9.40E−04 | 7/949 | 2.31E−03 | — | >0.05 |
| GO: 0023026 | MHC class II protein complex | 6/812 | 1.04E−03 | 6/949 | 2.32E−03 | — | >0.05 |
| | binding | | | | | | |
| GO: 0030027 | lamellipodium | 26/820 | 5.30E−07 | 25/959 | 2.69E−05 | 31/1866 | 7.11E−03 |
| GO: 0030032 | lamellipodium assembly | 12/810 | 7.73E−05 | 12/941 | 3.13E−04 | — | >0.05 |
| GO: 0030041 | actin filament polymerization | 25/810 | 4.05E−08 | 26/941 | 1.88E−07 | 31/1792 | 1.75E−04 |
| GO: 0030042 | actin filament depolymerization | 8/810 | 3.77E−03 | — | >0.05 | — | >0.05 |
| GO: 0030055 | cell-substrate junction | 88/820 | 1.47E−35 | 90/959 | 7.55E−32 | 102/1866 | 9.65E−18 |
| GO: 0030098 | lymphocyte differentiation | 41/810 | 1.85E−06 | 43/941 | 1.35E−05 | — | >0.05 |
| GO: 0030099 | myeloid cell differentiation | 57/810 | 2.82E−14 | 59/941 | 1.35E−12 | 73/1792 | 6.19E−07 |
| GO: 0030117 | membrane coat | 12/820 | 5.34E−04 | 16/959 | 1.17E−05 | 20/1866 | 4.51E−04 |
| GO: 0030118 | clathrin coat | 7/820 | 3.58E−03 | 8/959 | 2.09E−03 | 11/1866 | 4.59E−03 |
| GO: 0030120 | vesicle coat | 9/820 | 9.46E−04 | 13/959 | 6.43E−06 | 16/1866 | 1.40E−04 |
| GO: 0030125 | clathrin vesicle coat | 6/820 | 2.46E−03 | 7/959 | 1.03E−03 | 9/1866 | 3.38E−03 |
| GO: 0030126 | COPI vesicle coat | 3/820 | 1.61E−02 | 4/959 | 3.08E−03 | — | >0.05 |
| GO: 0030130 | clathrin coat of trans-Golgi network | 4/820 | 6.26E−03 | 5/959 | 1.53E−03 | — | >0.05 |
| | vesicle | | | | | | |
| GO: 0030132 | clathrin coat of coated pit | 5/820 | 4.13E−04 | 6/959 | 7.94E−05 | 7/1866 | 4.20E−04 |
| GO: 0030133 | transport vesicle | 32/820 | 1.38E−03 | 39/959 | 1.85E−04 | 66/1866 | 8.19E−05 |
| GO: 0030135 | coated vesicle | 27/820 | 4.01E−04 | 31/959 | 2.05E−04 | 44/1866 | 7.22E−03 |
| GO: 0030136 | clathrin-coated vesicle | 22/820 | 1.31E−04 | 23/959 | 4.58E−04 | 34/1866 | 2.86E−03 |
| GO: 0030139 | endocytic vesicle | 38/820 | 1.01E−07 | 43/959 | 3.48E−08 | 59/1866 | 1.35E−05 |
| GO: 0030140 | trans-Golgi network transport | 6/820 | 5.82E−03 | 7/959 | 2.87E−03 | — | >0.05 |
| | vesicle | | | | | | |
| GO: 0030159 | signaling receptor complex adaptor | 8/812 | 2.04E−03 | 8/949 | 5.29E−03 | — | >0.05 |
| | activity | | | | | | |
| GO: 0030175 | filopodium | 12/820 | 2.21E−03 | 12/959 | 7.64E−03 | — | >0.05 |
| GO: 0030183 | B cell differentiation | 20/810 | 1.35E−05 | 20/941 | 1.10E−04 | — | >0.05 |
| GO: 0030217 | T cell differentiation | 28/810 | 1.52E−04 | 30/941 | 3.55E−04 | — | >0.05 |
| GO: 0030218 | erythrocyte differentiation | 31/810 | 7.25E−15 | 32/941 | 6.47E−14 | 33/1792 | 2.08E−07 |
| GO: 0030219 | megakaryocyte differentiation | 11/810 | 1.17E−04 | 11/941 | 4.26E−04 | — | >0.05 |
| GO: 0030291 | protein serine/threonine kinase | 7/812 | 9.40E−04 | 7/949 | 2.31E−03 | — | >0.05 |
| | inhibitor activity | | | | | | |
| GO: 0030316 | osteoclast differentiation | — | >0.05 | 12/941 | 5.94E−03 | — | >0.05 |
| GO: 0030496 | midbody | 22/820 | 5.54E−05 | 23/959 | 1.98E−04 | — | >0.05 |
| GO: 0030500 | regulation of bone mineralization | — | >0.05 | — | >0.05 | 17/1792 | 1.38E−03 |
| GO: 0030511 | positive regulation of transforming | — | >0.05 | 7/941 | 1.45E−03 | — | >0.05 |
| | growth factor beta receptor | | | | | | |
| | signaling pathway | | | | | | |
| GO: 0030522 | intracellular receptor signaling | 32/810 | 2.86E−06 | 34/941 | 9.45E−06 | 46/1792 | 1.04E−03 |
| | pathway | | | | | | |
| GO: 0030527 | structural constituent of chromatin | 25/812 | 7.21E−14 | 26/949 | 3.04E−13 | 26/1815 | 3.37E−07 |
| GO: 0030593 | neutrophil chemotaxis | 14/810 | 2.31E−04 | 14/941 | 1.02E−03 | — | >0.05 |
| GO: 0030595 | leukocyte chemotaxis | 24/810 | 1.55E−04 | 26/941 | 2.47E−04 | — | >0.05 |
| GO: 0030643 | intracellular phosphate ion | — | >0.05 | — | >0.05 | 5/1792 | 1.46E−03 |
| | homeostasis | | | | | | |
| GO: 0030658 | transport vesicle membrane | 20/820 | 2.46E−03 | 26/959 | 1.10E−04 | 41/1866 | 1.31E−04 |
| GO: 0030660 | Golgi-associated vesicle membrane | 7/820 | 1.14E−02 | 9/959 | 2.19E−03 | — | >0.05 |
| GO: 0030662 | coated vesicle membrane | 20/820 | 3.98E−04 | 24/959 | 6.90E−05 | 33/1866 | 1.91E−03 |
| GO: 0030663 | COPI-coated vesicle membrane | — | >0.05 | 4/959 | 1.08E−02 | — | >0.05 |
| GO: 0030665 | clathrin-coated vesicle membrane | 16/820 | 2.11E−04 | 17/959 | 4.07E−04 | 25/1866 | 1.20E−03 |
| GO: 0030666 | endocytic vesicle membrane | 20/820 | 2.68E−04 | 23/959 | 1.17E−04 | 34/1866 | 5.60E−04 |
| GO: 0030667 | secretory granule membrane | 25/820 | 2.68E−03 | 26/959 | 9.97E−03 | — | >0.05 |
| GO: 0030669 | clathrin-coated endocytic vesicle | 8/820 | 1.15E−02 | 9/959 | 9.42E−03 | — | >0.05 |
| | membrane | | | | | | |
| GO: 0030670 | phagocytic vesicle membrane | 9/820 | 5.91E−03 | 10/959 | 5.32E−03 | — | >0.05 |
| GO: 0030672 | synaptic vesicle membrane | — | >0.05 | 14/959 | 3.59E−03 | 26/1866 | 1.28E−04 |
| GO: 0030674 | protein-macromolecule adaptor | 32/812 | 1.93E−04 | 34/949 | 6.88E−04 | — | >0.05 |
| | activity | | | | | | |
| GO: 0030695 | GTPase regulator activity | 36/812 | 2.90E−03 | — | >0.05 | — | >0.05 |
| GO: 0030832 | regulation of actin filament length | 19/810 | 3.77E−05 | 19/941 | 2.67E−04 | — | >0.05 |
| GO: 0030833 | regulation of actin filament | 19/810 | 3.80E−06 | 19/941 | 3.13E−05 | 24/1792 | 1.47E−03 |
| | polymerization | | | | | | |
| GO: 0030837 | negative regulation of actin filament | 11/810 | 1.01E−04 | 11/941 | 3.71E−04 | 15/1792 | 1.04E−03 |
| | polymerization | | | | | | |
| GO: 0030850 | prostate gland development | — | >0.05 | — | >0.05 | 12/1792 | 1.79E−03 |
| GO: 0030858 | positive regulation of epithelial cell | — | >0.05 | — | >0.05 | 15/1792 | 2.33E−03 |
| | differentiation | | | | | | |
| GO: 0030863 | cortical cytoskeleton | 18/820 | 3.88E−07 | 19/959 | 8.36E−07 | 23/1866 | 1.33E−04 |
| GO: 0030864 | cortical actin cytoskeleton | 12/820 | 5.67E−05 | 12/959 | 2.47E−04 | 15/1866 | 3.96E−03 |
| GO: 0030865 | cortical cytoskeleton organization | 9/810 | 2.16E−04 | 9/941 | 6.48E−04 | — | >0.05 |
| GO: 0030866 | cortical actin cytoskeleton | 6/810 | 7.71E−03 | — | >0.05 | — | >0.05 |
| | organization | | | | | | |
| GO: 0030867 | rough endoplasmic reticulum | — | >0.05 | 5/959 | 5.91E−03 | 7/1866 | 6.35E−03 |
| | membrane | | | | | | |
| GO: 0030879 | mammary gland development | — | >0.05 | 15/941 | 3.90E−03 | — | >0.05 |
| GO: 0030888 | regulation of B cell proliferation | 8/810 | 7.58E−03 | 9/941 | 5.69E−03 | — | >0.05 |
| GO: 0030889 | negative regulation of B cell | 5/810 | 6.56E−04 | 5/941 | 1.29E−03 | — | >0.05 |
| | proliferation | | | | | | |
| GO: 0030900 | forebrain development | — | >0.05 | — | >0.05 | 57/1792 | 1.85E−03 |
| GO: 0030904 | retromer complex | 3/820 | 1.28E−02 | — | >0.05 | — | >0.05 |
| GO: 0031053 | primary miRNA processing | 4/810 | 5.65E−03 | — | >0.05 | — | >0.05 |
| GO: 0031072 | heat shock protein binding | 15/812 | 5.59E−04 | 17/949 | 3.35E−04 | — | >0.05 |
| GO: 0031074 | nucleocytoplasmic transport | 3/820 | 1.28E−02 | — | >0.05 | — | >0.05 |
| | complex | | | | | | |
| GO: 0031098 | stress-activated protein kinase | 25/810 | 8.82E−05 | 25/941 | 8.35E−04 | — | >0.05 |
| | signaling cascade | | | | | | |
| GO: 0031105 | septin complex | — | >0.05 | — | >0.05 | 5/1866 | 8.16E−03 |
| GO: 0031209 | SCAR complex | 3/820 | 1.28E−02 | — | >0.05 | — | >0.05 |
| GO: 0031234 | extrinsic component of cytoplasmic | 13/820 | 2.77E−05 | 13/959 | 1.37E−04 | 17/1866 | 1.32E−03 |
| | side of plasma membrane | | | | | | |
| GO: 0031252 | cell leading edge | 45/820 | 1.55E−08 | 46/959 | 5.43E−07 | 67/1866 | 3.63E−05 |
| GO: 0031253 | cell projection membrane | 30/820 | 2.21E−04 | 32/959 | 6.79E−04 | 64/1866 | 4.16E−07 |
| GO: 0031256 | leading edge membrane | 20/820 | 8.34E−05 | 21/959 | 2.45E−04 | 36/1866 | 1.90E−05 |
| GO: 0031258 | lamellipodium membrane | 4/820 | 1.31E−02 | — | >0.05 | — | >0.05 |
| GO: 0031267 | small GTPase binding | 26/812 | 2.34E−04 | 26/949 | 2.28E−03 | — | >0.05 |
| GO: 0031294 | lymphocyte costimulation | 7/810 | 4.27E−03 | — | >0.05 | — | >0.05 |
| GO: 0031295 | T cell costimulation | 7/810 | 3.33E−03 | 7/941 | 7.50E−03 | — | >0.05 |
| GO: 0031330 | negative regulation of cellular | 34/810 | 5.25E−09 | 36/941 | 1.98E−08 | 46/1792 | 1.85E−05 |
| | catabolic process | | | | | | |
| GO: 0031331 | positive regulation of cellular | 42/810 | 3.67E−06 | 43/941 | 5.93E−05 | — | >0.05 |
| | catabolic process | | | | | | |
| GO: 0031333 | negative regulation of protein- | 18/810 | 7.83E−05 | 20/941 | 5.66E−05 | 29/1792 | 1.59E−04 |
| | containing complex assembly | | | | | | |
| GO: 0031334 | positive regulation of protein- | 25/810 | 1.68E−06 | 26/941 | 7.64E−06 | — | >0.05 |
| | containing complex assembly | | | | | | |
| GO: 0031341 | regulation of cell killing | 12/810 | 3.35E−03 | — | >0.05 | — | >0.05 |
| GO: 0031346 | positive regulation of cell projection | 28/810 | 1.98E−03 | — | >0.05 | — | >0.05 |
| | organization | | | | | | |
| GO: 0031348 | negative regulation of defense | 26/810 | 3.48E−04 | 30/941 | 1.36E−04 | — | >0.05 |
| | response | | | | | | |
| GO: 0031349 | positive regulation of defense | 47/810 | 1.88E−08 | 49/941 | 2.58E−07 | 64/1792 | 7.26E−04 |
| | response | | | | | | |
| GO: 0031369 | translation initiation factor binding | 6/812 | 2.64E−03 | — | >0.05 | — | >0.05 |
| GO: 0031386 | protein tag | 5/812 | 1.69E−04 | 5/949 | 3.51E−04 | — | >0.05 |
| GO: 0031396 | regulation of protein ubiquitination | 33/810 | 2.03E−10 | 34/941 | 2.36E−09 | 37/1792 | 2.88E−04 |
| GO: 0031397 | negative regulation of protein | 18/810 | 2.04E−08 | 18/941 | 1.95E−07 | 20/1792 | 1.31E−04 |
| | ubiquitination | | | | | | |
| GO: 0031398 | positive regulation of protein | 13/810 | 2.28E−03 | 14/941 | 3.10E−03 | — | >0.05 |
| | ubiquitination | | | | | | |
| GO: 0031430 | M band | — | >0.05 | 4/959 | 1.58E−02 | — | >0.05 |
| GO: 0031464 | Cul4A-RING E3 ubiquitin ligase | 3/820 | 1.61E−02 | — | >0.05 | — | >0.05 |
| | complex | | | | | | |
| GO: 0031468 | nuclear membrane reassembly | 7/810 | 7.59E−05 | 7/941 | 1.94E−04 | — | >0.05 |
| GO: 0031490 | chromatin DNA binding | 20/812 | 2.68E−07 | 20/949 | 3.01E−06 | 23/1815 | 1.33E−03 |
| GO: 0031491 | nucleosome binding | 16/812 | 7.15E−08 | 16/949 | 5.89E−07 | 16/1815 | 1.47E−03 |
| GO: 0031492 | nucleosomal DNA binding | 13/812 | 1.45E−08 | 13/949 | 9.11E−08 | 13/1815 | 1.16E−04 |
| GO: 0031529 | ruffle organization | 8/810 | 2.69E−03 | 8/941 | 6.62E−03 | — | >0.05 |
| GO: 0031593 | polyubiquitin modification- | 9/812 | 6.90E−04 | 9/949 | 2.05E−03 | — | >0.05 |
| | dependent protein binding | | | | | | |
| GO: 0031623 | receptor internalization | 17/810 | 3.17E−05 | 19/941 | 1.77E−05 | 28/1792 | 1.88E−05 |
| GO: 0031625 | ubiquitin protein ligase binding | 46/812 | 1.96E−13 | 44/949 | 4.96E−10 | 55/1815 | 6.25E−06 |
| GO: 0031640 | killing of cells of another organism | 8/810 | 1.26E−03 | 8/941 | 3.21E−03 | — | >0.05 |
| GO: 0031647 | regulation of protein stability | 34/810 | 1.94E−06 | 35/941 | 1.90E−05 | — | >0.05 |
| GO: 0031668 | cellular response to extracellular | 23/810 | 1.12E−03 | 26/941 | 8.00E−04 | — | >0.05 |
| | stimulus | | | | | | |
| GO: 0031669 | cellular response to nutrient levels | 19/810 | 6.05E−03 | — | >0.05 | — | >0.05 |
| GO: 0031690 | adrenergic receptor binding | — | >0.05 | 5/949 | 1.88E−03 | — | >0.05 |
| GO: 0031838 | haptoglobin-hemoglobin complex | — | >0.05 | 3/959 | 1.52E−02 | — | >0.05 |
| GO: 0031901 | early endosome membrane | 17/820 | 2.49E−03 | 18/959 | 5.32E−03 | — | >0.05 |
| GO: 0031902 | late endosome membrane | 17/820 | 3.20E−04 | 17/959 | 1.81E−03 | — | >0.05 |
| GO: 0031968 | organelle outer membrane | 18/820 | 1.23E−02 | — | >0.05 | — | >0.05 |
| GO: 0031970 | organelle envelope lumen | 10/820 | 6.30E−03 | 11/959 | 6.58E−03 | — | >0.05 |
| GO: 0031983 | vesicle lumen | 49/820 | 1.54E−14 | 51/959 | 3.95E−13 | 59/1866 | 2.21E−06 |
| GO: 0032045 | guanyl-nucleotide exchange factor | 4/820 | 5.03E−03 | 4/959 | 8.73E−03 | — | >0.05 |
| | complex | | | | | | |
| GO: 0032092 | positive regulation of protein | 14/810 | 2.56E−05 | 14/941 | 1.29E−04 | — | >0.05 |
| | binding | | | | | | |
| GO: 0032102 | negative regulation of response to | 32/810 | 4.74E−03 | 38/941 | 1.48E−03 | — | >0.05 |
| | external stimulus | | | | | | |
| GO: 0032200 | telomere organization | 23/810 | 7.99E−06 | 24/941 | 2.93E−05 | — | >0.05 |
| GO: 0032210 | regulation of telomere maintenance | 7/810 | 8.35E−03 | — | >0.05 | — | >0.05 |
| | via telomerase | | | | | | |
| GO: 0032212 | positive regulation of telomere | 6/810 | 3.38E−03 | 6/941 | 7.00E−03 | — | >0.05 |
| | maintenance via telomerase | | | | | | |
| GO: 0032271 | regulation of protein polymerization | 25/810 | 1.04E−06 | 25/941 | 1.43E−05 | 32/1792 | 1.55E−03 |
| GO: 0032272 | negative regulation of protein | 13/810 | 3.88E−05 | 13/941 | 1.76E−04 | 18/1792 | 4.95E−04 |
| | polymerization | | | | | | |
| GO: 0032273 | positive regulation of protein | 11/810 | 1.66E−03 | 11/941 | 5.24E−03 | — | >0.05 |
| | polymerization | | | | | | |
| GO: 0032279 | asymmetric synapse | 33/820 | 2.11E−05 | 37/959 | 1.65E−05 | 71/1866 | 1.90E−09 |
| GO: 0032331 | negative regulation of chondrocyte | — | >0.05 | 5/941 | 6.59E−03 | 8/1792 | 1.43E−03 |
| | differentiation | | | | | | |
| GO: 0032355 | response to estradiol | 12/810 | 6.75E−03 | 15/941 | 1.22E−03 | 23/1792 | 1.21E−03 |
| GO: 0032386 | regulation of intracellular transport | 33/810 | 1.14E−05 | 37/941 | 7.25E−06 | — | >0.05 |
| GO: 0032388 | positive regulation of intracellular | 18/810 | 2.70E−03 | 22/941 | 4.69E−04 | — | >0.05 |
| | transport | | | | | | |
| GO: 0032432 | actin filament bundle | 10/820 | 1.57E−03 | 10/959 | 4.85E−03 | 16/1866 | 2.64E−03 |
| GO: 0032434 | regulation of proteasomal ubiquitin- | 19/810 | 1.15E−05 | 18/941 | 2.68E−04 | — | >0.05 |
| | dependent protein catabolic | | | | | | |
| | process | | | | | | |
| GO: 0032435 | negative regulation of proteasomal | 9/810 | 3.12E−05 | 8/941 | 5.73E−04 | 11/1792 | 7.43E−04 |
| | ubiquitin-dependent protein | | | | | | |
| | catabolic process | | | | | | |
| GO: 0032436 | positive regulation of proteasomal | 10/810 | 6.75E−03 | — | >0.05 | — | >0.05 |
| | ubiquitin-dependent protein | | | | | | |
| | catabolic process | | | | | | |
| GO: 0032479 | regulation of type I interferon | 12/810 | 1.77E−03 | 12/941 | 5.94E−03 | — | >0.05 |
| | production | | | | | | |
| GO: 0032481 | positive regulation of type I | 8/810 | 4.66E−03 | — | >0.05 | — | >0.05 |
| | interferon production | | | | | | |
| GO: 0032495 | response to muramyl dipeptide | 6/810 | 1.65E−04 | 6/941 | 3.72E−04 | 7/1792 | 2.06E−03 |
| GO: 0032496 | response to lipopolysaccharide | 27/810 | 2.84E−03 | — | >0.05 | — | >0.05 |
| GO: 0032506 | cytokinetic process | 7/810 | 1.65E−03 | 7/941 | 3.84E−03 | — | >0.05 |
| GO: 0032509 | endosome transport via | 8/810 | 3.63E−04 | 8/941 | 9.76E−04 | — | >0.05 |
| | multivesicular body sorting pathway | | | | | | |
| GO: 0032510 | endosome to lysosome transport | 8/810 | 2.37E−07 | 8/941 | 7.43E−07 | 8/1792 | 8.79E−05 |
| | via multivesicular body sorting | | | | | | |
| | pathway | | | | | | |
| GO: 0032515 | negative regulation of | 5/810 | 3.49E−03 | 5/941 | 6.59E−03 | 8/1792 | 1.43E−03 |
| | phosphoprotein phosphatase | | | | | | |
| | activity | | | | | | |
| GO: 0032528 | microvillus organization | 5/810 | 3.49E−03 | 5/941 | 6.59E−03 | — | >0.05 |
| GO: 0032535 | regulation of cellular component | 37/810 | 1.04E−06 | 40/941 | 2.22E−06 | 51/1792 | 2.88E−03 |
| | size | | | | | | |
| GO: 0032561 | guanyl ribonucleotide binding | 41/812 | 6.26E−07 | 43/949 | 5.48E−06 | 59/1815 | 1.38E−03 |
| GO: 0032585 | multivesicular body membrane | 6/820 | 1.22E−03 | 6/959 | 2.70E−03 | — | >0.05 |
| GO: 0032587 | ruffle membrane | 15/820 | 2.16E−05 | 15/959 | 1.27E−04 | 22/1866 | 2.00E−04 |
| GO: 0032602 | chemokine production | 12/810 | 1.37E−03 | — | >0.05 | — | >0.05 |
| GO: 0032606 | type I interferon production | 12/810 | 1.77E−03 | 12/941 | 5.94E−03 | — | >0.05 |
| GO: 0032615 | interleukin-12 production | 10/810 | 4.61E−04 | 11/941 | 3.71E−04 | — | >0.05 |
| GO: 0032635 | interleukin-6 production | 21/810 | 3.36E−05 | 20/941 | 7.13E−04 | — | >0.05 |
| GO: 0032640 | tumor necrosis factor production | 19/810 | 5.15E−04 | 18/941 | 6.52E−03 | — | >0.05 |
| GO: 0032642 | regulation of chemokine production | 12/810 | 1.25E−03 | — | >0.05 | — | >0.05 |
| GO: 0032655 | regulation of interleukin-12 | 10/810 | 4.61E−04 | 11/941 | 3.71E−04 | — | >0.05 |
| | production | | | | | | |
| GO: 0032675 | regulation of interleukin-6 | 21/810 | 3.36E−05 | 20/941 | 7.13E−04 | — | >0.05 |
| | production | | | | | | |
| GO: 0032680 | regulation of tumor necrosis factor | 19/810 | 5.15E−04 | 18/941 | 6.52E−03 | — | >0.05 |
| | production | | | | | | |
| GO: 0032695 | negative regulation of interleukin-12 | 4/810 | 8.58E−03 | — | >0.05 | — | >0.05 |
| | production | | | | | | |
| GO: 0032722 | positive regulation of chemokine | 9/810 | 3.54E−03 | — | >0.05 | — | >0.05 |
| | production | | | | | | |
| GO: 0032755 | positive regulation of interleukin-6 | 11/810 | 4.24E−03 | — | >0.05 | — | >0.05 |
| | production | | | | | | |
| GO: 0032760 | positive regulation of tumor | 12/810 | 2.09E−03 | — | >0.05 | — | >0.05 |
| | necrosis factor production | | | | | | |
| GO: 0032794 | GTPase activating protein binding | 4/812 | 3.66E−03 | — | >0.05 | — | >0.05 |
| GO: 0032809 | neuronal cell body membrane | — | >0.05 | — | >0.05 | 8/1866 | 5.22E−03 |
| GO: 0032868 | response to insulin | 23/810 | 2.24E−03 | 26/941 | 1.72E−03 | 42/1792 | 1.90E−03 |
| GO: 0032869 | cellular response to insulin stimulus | 20/810 | 1.10E−03 | 22/941 | 1.26E−03 | — | >0.05 |
| GO: 0032872 | regulation of stress-activated MAPK | 20/810 | 3.54E−04 | 19/941 | 4.93E−03 | — | >0.05 |
| | cascade | | | | | | |
| GO: 0032873 | negative regulation of stress- | 9/810 | 3.48E−04 | 9/941 | 1.02E−03 | — | >0.05 |
| | activated MAPK cascade | | | | | | |
| GO: 0032886 | regulation of microtubule-based | 21/810 | 3.68E−03 | 23/941 | 4.93E−03 | — | >0.05 |
| | process | | | | | | |
| GO: 0032928 | regulation of superoxide anion | 7/810 | 3.02E−05 | 7/941 | 7.87E−05 | 8/1792 | 7.41E−04 |
| | generation | | | | | | |
| GO: 0032930 | positive regulation of superoxide | 7/810 | 1.00E−05 | 7/941 | 2.66E−05 | 8/1792 | 2.29E−04 |
| | anion generation | | | | | | |
| GO: 0032943 | mononuclear cell proliferation | 35/810 | 4.12E−07 | 37/941 | 1.82E−06 | 48/1792 | 1.02E−03 |
| GO: 0032944 | regulation of mononuclear cell | 29/810 | 1.05E−06 | 31/941 | 2.49E−06 | 41/1792 | 3.30E−04 |
| | proliferation | | | | | | |
| GO: 0032945 | negative regulation of mononuclear | 11/810 | 1.82E−03 | 12/941 | 1.91E−03 | 19/1792 | 8.26E−04 |
| | cell proliferation | | | | | | |
| GO: 0032946 | positive regulation of mononuclear | 17/810 | 2.68E−04 | 18/941 | 5.34E−04 | — | >0.05 |
| | cell proliferation | | | | | | |
| GO: 0032956 | regulation of actin cytoskeleton | 29/810 | 7.09E−04 | 30/941 | 3.40E−03 | — | >0.05 |
| | organization | | | | | | |
| GO: 0032970 | regulation of actin filament-based | 31/810 | 1.00E−03 | 33/941 | 2.87E−03 | — | >0.05 |
| | process | | | | | | |
| GO: 0032984 | protein-containing complex | 30/810 | 2.09E−07 | 30/941 | 4.59E−06 | — | >0.05 |
| | disassembly | | | | | | |
| GO: 0032993 | protein-DNA complex | 34/820 | 4.99E−11 | 36/959 | 2.00E−10 | 39/1866 | 1.20E−04 |
| GO: 0033002 | muscle cell proliferation | 20/810 | 7.04E−03 | — | >0.05 | — | >0.05 |
| GO: 0033003 | regulation of mast cell activation | — | >0.05 | 7/941 | 6.62E−03 | — | >0.05 |
| GO: 0033006 | regulation of mast cell activation | 6/810 | 2.89E−03 | 6/941 | 6.02E−03 | — | >0.05 |
| | involved in immune response | | | | | | |
| GO: 0033007 | negative regulation of mast cell | 3/810 | 8.14E−03 | — | >0.05 | — | >0.05 |
| | activation involved in immune | | | | | | |
| | response | | | | | | |
| GO: 0033077 | T cell differentiation in thymus | 11/810 | 1.25E−03 | 13/941 | 3.72E−04 | 19/1792 | 4.52E−04 |
| GO: 0033119 | negative regulation of RNA splicing | 8/810 | 1.46E−05 | 8/941 | 4.29E−05 | 10/1792 | 1.36E−04 |
| GO: 0033120 | positive regulation of RNA splicing | 8/810 | 7.01E−04 | 8/941 | 1.84E−03 | — | >0.05 |
| GO: 0033157 | regulation of intracellular protein | 23/810 | 1.42E−04 | 26/941 | 8.01E−05 | — | >0.05 |
| | transport | | | | | | |
| GO: 0033176 | proton-transporting V-type ATPase | — | >0.05 | 5/959 | 9.92E−03 | — | >0.05 |
| | complex | | | | | | |
| GO: 0033177 | proton-transporting two-sector | — | >0.05 | 6/959 | 9.47E−04 | — | >0.05 |
| | ATPase complex, proton- | | | | | | |
| | transporting domain | | | | | | |
| GO: 0033178 | proton-transporting two-sector | 6/820 | 1.00E−04 | 7/959 | 2.25E−05 | 7/1866 | 1.42E−03 |
| | ATPase complex, catalytic domain | | | | | | |
| GO: 0033209 | tumor necrosis factor-mediated | 11/810 | 8.69E−03 | — | >0.05 | — | >0.05 |
| | signaling pathway | | | | | | |
| GO: 0033290 | eukaryotic 48S preinitiation | 7/820 | 1.20E−06 | 7/959 | 3.43E−06 | 8/1866 | 2.70E−05 |
| | complex | | | | | | |
| GO: 0033628 | regulation of cell adhesion | 7/810 | 5.41E−03 | — | >0.05 | — | >0.05 |
| | mediated by integrin | | | | | | |
| GO: 0033673 | negative regulation of kinase | 33/810 | 5.40E−09 | 34/941 | 5.95E−08 | 47/1792 | 2.09E−06 |
| | activity | | | | | | |
| GO: 0034063 | stress granule assembly | 6/810 | 1.19E−03 | 6/941 | 2.55E−03 | — | >0.05 |
| GO: 0034101 | erythrocyte homeostasis | 35/810 | 2.05E−17 | 36/941 | 3.05E−16 | 37/1792 | 9.93E−09 |
| GO: 0034219 | carbohydrate transmembrane | — | >0.05 | — | >0.05 | 24/1792 | 2.03E−03 |
| | transport | | | | | | |
| GO: 0034236 | protein kinase A catalytic subunit | 4/812 | 2.06E−03 | 5/949 | 3.51E−04 | — | >0.05 |
| | binding | | | | | | |
| GO: 0034250 | positive regulation of amide | 19/810 | 1.74E−04 | 19/941 | 1.09E−03 | — | >0.05 |
| | metabolic process | | | | | | |
| GO: 0034314 | Arp2/3 complex-mediated actin | 7/810 | 3.78E−03 | 8/941 | 2.13E−03 | — | >0.05 |
| | nucleation | | | | | | |
| GO: 0034341 | response to type II interferon | 18/810 | 3.70E−05 | 19/941 | 8.03E−05 | — | >0.05 |
| GO: 0034399 | nuclear periphery | 14/820 | 4.19E−03 | 16/959 | 2.80E−03 | — | >0.05 |
| GO: 0034502 | protein localization to chromosome | 16/810 | 4.47E−05 | 16/941 | 2.54E−04 | — | >0.05 |
| GO: 0034504 | protein localization to nucleus | 33/810 | 1.83E−06 | 36/941 | 2.58E−06 | 48/1792 | 5.74E−04 |
| GO: 0034506 | chromosome, centromeric core | 8/820 | 3.20E−07 | 8/959 | 1.05E−06 | 8/1866 | 1.40E−04 |
| | domain | | | | | | |
| GO: 0034599 | cellular response to oxidative stress | 31/810 | 4.79E−06 | 33/941 | 1.44E−05 | — | >0.05 |
| GO: 0034612 | response to tumor necrosis factor | 22/810 | 2.15E−03 | 24/941 | 3.10E−03 | — | >0.05 |
| GO: 0034643 | establishment of mitochondrion | 5/810 | 5.94E−03 | — | >0.05 | — | >0.05 |
| | localization, microtubule-mediated | | | | | | |
| GO: 0034655 | nucleobase-containing compound | 42/810 | 2.76E−06 | 45/941 | 9.83E−06 | — | >0.05 |
| | catabolic process | | | | | | |
| GO: 0034702 | ion channel complex | — | >0.05 | — | >0.05 | 44/1866 | 3.53E−03 |
| GO: 0034703 | cation channel complex | — | >0.05 | — | >0.05 | 31/1866 | 3.33E−03 |
| GO: 0034709 | methylosome | 3/820 | 1.61E−02 | — | >0.05 | — | >0.05 |
| GO: 0034728 | nucleosome organization | 26/810 | 1.85E−10 | 28/941 | 1.88E−10 | 30/1792 | 1.35E−05 |
| GO: 0034765 | regulation of monoatomic ion | — | >0.05 | — | >0.05 | 66/1792 | 2.33E−03 |
| | transmembrane transport | | | | | | |
| GO: 0034774 | secretory granule lumen | 49/820 | 8.32E−15 | 51/959 | 2.14E−13 | 59/1866 | 1.32E−06 |
| GO: 0035035 | histone acetyltransferase binding | — | >0.05 | 6/949 | 9.46E−04 | — | >0.05 |
| GO: 0035115 | embryonic forelimb morphogenesis | — | >0.05 | — | >0.05 | 9/1792 | 2.77E−03 |
| GO: 0035116 | embryonic hindlimb morphogenesis | — | >0.05 | — | >0.05 | 9/1792 | 9.75E−04 |
| GO: 0035136 | forelimb morphogenesis | — | >0.05 | — | >0.05 | 11/1792 | 1.19E−03 |
| GO: 0035137 | hindlimb morphogenesis | — | >0.05 | — | >0.05 | 11/1792 | 3.39E−04 |
| GO: 0035196 | miRNA processing | 9/810 | 1.83E−04 | 9/941 | 5.51E−04 | 12/1792 | 1.47E−03 |
| GO: 0035198 | miRNA binding | 7/812 | 9.40E−04 | 7/949 | 2.31E−03 | — | >0.05 |
| GO: 0035264 | multicellular organism growth | 15/810 | 1.65E−03 | 18/941 | 3.81E−04 | — | >0.05 |
| GO: 0035303 | regulation of dephosphorylation | 13/810 | 3.77E−03 | 15/941 | 2.00E−03 | — | >0.05 |
| GO: 0035304 | regulation of protein | 10/810 | 4.92E−03 | 12/941 | 1.57E−03 | — | >0.05 |
| | dephosphorylation | | | | | | |
| GO: 0035305 | negative regulation of | 8/810 | 8.16E−04 | 8/941 | 2.13E−03 | 12/1792 | 1.20E−03 |
| | dephosphorylation | | | | | | |
| GO: 0035308 | negative regulation of protein | 6/810 | 2.45E−03 | 6/941 | 5.14E−03 | 9/1792 | 2.77E−03 |
| | dephosphorylation | | | | | | |
| GO: 0035577 | azurophil granule membrane | 7/820 | 1.14E−02 | — | >0.05 | — | >0.05 |
| GO: 0035578 | azurophil granule lumen | 17/820 | 2.61E−07 | 18/959 | 4.76E−07 | 19/1866 | 1.04E−03 |
| GO: 0035580 | specific granule lumen | 9/820 | 1.20E−03 | 9/959 | 3.49E−03 | — | >0.05 |
| GO: 0035591 | signaling adaptor activity | 11/812 | 1.15E−03 | 11/949 | 3.89E−03 | — | >0.05 |
| GO: 0035770 | ribonucleoprotein granule | 33/820 | 5.71E−08 | 34/959 | 6.88E−07 | 47/1866 | 5.81E−05 |
| GO: 0035855 | megakaryocyte development | 5/810 | 2.86E−03 | 5/941 | 5.44E−03 | — | >0.05 |
| GO: 0035861 | site of doublE-strand break | 10/820 | 1.74E−03 | 11/959 | 1.65E−03 | — | >0.05 |
| GO: 0035891 | exit from host cell | 10/810 | 2.47E−07 | 10/941 | 9.80E−07 | 11/1792 | 4.87E−05 |
| GO: 0035914 | skeletal muscle cell differentiation | — | >0.05 | 11/941 | 5.57E−04 | 16/1792 | 5.76E−04 |
| GO: 0036003 | positive regulation of transcription | 5/810 | 8.75E−04 | 5/941 | 1.71E−03 | — | >0.05 |
| | from RNA polymerase II promoter | | | | | | |
| | in response to stress | | | | | | |
| GO: 0036010 | protein localization to endosome | 7/810 | 1.30E−04 | 7/941 | 3.27E−04 | — | >0.05 |
| GO: 0036019 | endolysosome | 7/820 | 1.73E−04 | 7/959 | 4.50E−04 | 8/1866 | 5.22E−03 |
| GO: 0036020 | endolysosome membrane | 4/820 | 9.26E−03 | 4/959 | 1.58E−02 | — | >0.05 |
| GO: 0036230 | granulocyte activation | 9/810 | 2.16E−04 | 9/941 | 6.48E−04 | — | >0.05 |
| GO: 0036257 | multivesicular body organization | 7/810 | 4.06E−04 | 7/941 | 9.91E−04 | — | >0.05 |
| GO: 0036258 | multivesicular body assembly | 7/810 | 3.29E−04 | 7/941 | 8.10E−04 | — | >0.05 |
| GO: 0036293 | response to decreased oxygen | 27/810 | 4.16E−04 | 29/941 | 8.95E−04 | 46/1792 | 1.58E−03 |
| | levels | | | | | | |
| GO: 0036294 | cellular response to decreased | 20/810 | 1.64E−05 | 20/941 | 1.32E−04 | 29/1792 | 4.58E−04 |
| | oxygen levels | | | | | | |
| GO: 0036464 | cytoplasmic ribonucleoprotein | 32/820 | 3.67E−08 | 33/959 | 4.08E−07 | 46/1866 | 2.06E−05 |
| | granule | | | | | | |
| GO: 0036473 | cell death in response to oxidative | 13/810 | 4.05E−04 | 14/941 | 5.12E−04 | — | >0.05 |
| | stress | | | | | | |
| GO: 0036475 | neuron death in response to | 7/810 | 6.01E−04 | 7/941 | 1.45E−03 | — | >0.05 |
| | oxidative stress | | | | | | |
| GO: 0038061 | NIK/NF-kappaB signaling | 15/810 | 1.43E−03 | 16/941 | 2.37E−03 | — | >0.05 |
| GO: 0038066 | p38MAPK cascade | 8/810 | 3.02E−03 | 9/941 | 2.03E−03 | — | >0.05 |
| GO: 0038093 | Fc receptor signaling pathway | 10/810 | 9.19E−05 | 10/941 | 3.11E−04 | — | >0.05 |
| GO: 0038094 | Fc-gamma receptor signaling | 8/810 | 7.21E−05 | 8/941 | 2.04E−04 | — | >0.05 |
| | pathway | | | | | | |
| GO: 0038096 | Fc-gamma receptor signaling | 7/810 | 9.99E−05 | 7/941 | 2.53E−04 | — | >0.05 |
| | pathway involved in phagocytosis | | | | | | |
| GO: 0039529 | RIG-I signaling pathway | 8/810 | 7.68E−06 | 9/941 | 2.28E−06 | 9/1792 | 3.78E−04 |
| GO: 0039531 | regulation of viral-induced | 13/810 | 4.51E−07 | 14/941 | 3.73E−07 | 14/1792 | 5.16E−04 |
| | cytoplasmic pattern recognition | | | | | | |
| | receptor signaling pathway | | | | | | |
| GO: 0039532 | negative regulation of viral-induced | 6/810 | 1.65E−04 | 6/941 | 3.72E−04 | — | >0.05 |
| | cytoplasmic pattern recognition | | | | | | |
| | receptor signaling pathway | | | | | | |
| GO: 0039535 | regulation of RIG-I signaling | 6/810 | 1.65E−04 | 7/941 | 3.91E−05 | 7/1792 | 2.06E−03 |
| | pathway | | | | | | |
| GO: 0039694 | viral RNA genome replication | 5/810 | 8.12E−03 | — | >0.05 | — | >0.05 |
| GO: 0039702 | viral budding via host ESCRT | 6/810 | 3.83E−04 | 6/941 | 8.49E−04 | — | >0.05 |
| | complex | | | | | | |
| GO: 0040014 | regulation of multicellular organism | 8/810 | 6.91E−03 | 10/941 | 1.46E−03 | — | >0.05 |
| | growth | | | | | | |
| GO: 0040019 | positive regulation of embryonic | — | >0.05 | 6/941 | 8.49E−04 | 8/1792 | 1.04E−03 |
| | development | | | | | | |
| GO: 0040029 | epigenetic regulation of gene | 17/810 | 4.08E−03 | — | >0.05 | — | >0.05 |
| | expression | | | | | | |
| GO: 0042053 | regulation of dopamine metabolic | — | >0.05 | — | >0.05 | 7/1792 | 2.06E−03 |
| | process | | | | | | |
| GO: 0042060 | wound healing | 32/810 | 3.88E−03 | 36/941 | 3.79E−03 | — | >0.05 |
| GO: 0042069 | regulation of catecholamine | — | >0.05 | — | >0.05 | 7/1792 | 2.06E−03 |
| | metabolic process | | | | | | |
| GO: 0042098 | T cell proliferation | 25/810 | 7.64E−06 | 26/941 | 3.39E−05 | 36/1792 | 7.06E−04 |
| GO: 0042102 | positive regulation of T cell | 13/810 | 6.54E−04 | 13/941 | 2.52E−03 | — | >0.05 |
| | proliferation | | | | | | |
| GO: 0042113 | B cell activation | 30/810 | 4.50E−06 | 31/941 | 3.10E−05 | — | >0.05 |
| GO: 0042116 | macrophage activation | 12/810 | 3.11E−03 | — | >0.05 | — | >0.05 |
| GO: 0042119 | neutrophil activation | 8/810 | 4.31E−04 | 8/941 | 1.15E−03 | — | >0.05 |
| GO: 0042129 | regulation of T cell proliferation | 20/810 | 1.62E−04 | 21/941 | 4.33E−04 | — | >0.05 |
| GO: 0042176 | regulation of protein catabolic | 39/810 | 1.96E−07 | 40/941 | 3.16E−06 | — | >0.05 |
| | process | | | | | | |
| GO: 0042177 | negative regulation of protein | 13/810 | 1.21E−03 | 13/941 | 4.46E−03 | — | >0.05 |
| | catabolic process | | | | | | |
| GO: 0042254 | ribosome biogenesis | 27/810 | 2.87E−04 | 27/941 | 2.63E−03 | — | >0.05 |
| GO: 0042255 | ribosome assembly | 11/810 | 4.64E−05 | 11/941 | 1.76E−04 | — | >0.05 |
| GO: 0042267 | natural killer cell mediated | 10/810 | 1.81E−03 | 10/941 | 5.28E−03 | — | >0.05 |
| | cytotoxicity | | | | | | |
| GO: 0042273 | ribosomal large subunit biogenesis | 10/810 | 8.49E−04 | 10/941 | 2.59E−03 | — | >0.05 |
| GO: 0042274 | ribosomal small subunit biogenesis | 11/810 | 4.80E−04 | 11/941 | 1.63E−03 | — | >0.05 |
| GO: 0042287 | MHC protein binding | 7/812 | 2.41E−03 | — | >0.05 | — | >0.05 |
| GO: 0042306 | regulation of protein import into | — | >0.05 | 9/941 | 2.60E−03 | — | >0.05 |
| | nucleus | | | | | | |
| GO: 0042307 | positive regulation of protein import | 6/810 | 6.80E−03 | 8/941 | 6.88E−04 | — | >0.05 |
| | into nucleus | | | | | | |
| GO: 0042326 | negative regulation of | 43/810 | 1.78E−08 | 45/941 | 1.72E−07 | 69/1792 | 3.49E−07 |
| | phosphorylation | | | | | | |
| GO: 0042393 | histone binding | 20/812 | 5.77E−03 | — | >0.05 | — | >0.05 |
| GO: 0042470 | melanosome | 21/820 | 9.74E−09 | 21/959 | 1.41E−07 | 23/186 | 4.19E−04 |
| | | | | | | 6 | |
| GO: 0042541 | hemoglobin biosynthetic process | 6/810 | 1.60E−05 | 6/941 | 3.76E−05 | 6/1792 | 1.29E−03 |
| GO: 0042542 | response to hydrogen peroxide | 14/810 | 1.45E−03 | 16/941 | 7.81E−04 | — | >0.05 |
| GO: 0042554 | superoxide anion generation | 9/810 | 8.85E−05 | 10/941 | 4.88E−05 | 12/1792 | 6.23E−04 |
| GO: 0042581 | specific granule | 16/820 | 1.39E−03 | 16/959 | 6.42E−03 | — | >0.05 |
| GO: 0042582 | azurophil granule | 24/820 | 4.37E−08 | 25/959 | 2.04E−07 | 29/1866 | 4.27E−04 |
| GO: 0042583 | chromaffin granule | — | >0.05 | — | >0.05 | 5/1866 | 5.69E−03 |
| GO: 0042611 | MHC protein complex | 5/820 | 3.66E−03 | 5/959 | 7.09E−03 | — | >0.05 |
| GO: 0042613 | MHC class II protein complex | 4/820 | 5.03E−03 | 4/959 | 8.73E−03 | — | >0.05 |
| GO: 0042625 | ATPase-coupled ion | — | >0.05 | 6/949 | 1.21E−03 | — | >0.05 |
| | transmembrane transporter activity | | | | | | |
| GO: 0042641 | actomyosin | 8/820 | 1.56E−02 | — | >0.05 | — | >0.05 |
| GO: 0042742 | defense response to bacterium | 24/810 | 6.05E−03 | — | >0.05 | — | >0.05 |
| GO: 0042743 | hydrogen peroxide metabolic | 9/810 | 5.41E−04 | 10/941 | 3.65E−04 | — | >0.05 |
| | process | | | | | | |
| GO: 0042752 | regulation of circadian rhythm | — | >0.05 | 13/941 | 5.59E−03 | — | >0.05 |
| GO: 0042771 | intrinsic apoptotic signaling pathway | 8/810 | 5.99E−04 | 8/941 | 1.58E−03 | — | >0.05 |
| | in response to DNA damage by p53 | | | | | | |
| | class mediator | | | | | | |
| GO: 0042773 | ATP synthesis coupled electron | 13/810 | 1.55E−04 | 13/941 | 6.53E−04 | — | >0.05 |
| | transport | | | | | | |
| GO: 0042775 | mitochondrial ATP synthesis | 13/810 | 1.55E−04 | 13/941 | 6.53E−04 | — | >0.05 |
| | coupled electron transport | | | | | | |
| GO: 0042776 | proton motive force-driven | 9/810 | 9.25E−04 | 10/941 | 6.61E−04 | — | >0.05 |
| | mitochondrial ATP synthesis | | | | | | |
| GO: 0042788 | polysomal ribosome | 16/820 | 1.75E−14 | 16/959 | 1.97E−13 | 18/1866 | 3.31E−11 |
| GO: 0042826 | histone deacetylase binding | 14/812 | 1.32E−03 | 17/949 | 2.51E−04 | — | >0.05 |
| GO: 0043010 | camera-type eye development | — | >0.05 | — | >0.05 | 57/1792 | 2.92E−05 |
| GO: 0043020 | NADPH oxidase complex | 4/820 | 6.26E−03 | 4/959 | 1.08E−02 | — | >0.05 |
| GO: 0043021 | ribonucleoprotein complex binding | 18/812 | 2.79E−04 | 18/949 | 1.69E−03 | — | >0.05 |
| GO: 0043025 | neuronal cell body | — | >0.05 | — | >0.05 | 71/1866 | 6.84E−04 |
| GO: 0043029 | T cell homeostasis | — | >0.05 | 7/941 | 5.82E−03 | — | >0.05 |
| GO: 0043112 | receptor metabolic process | 9/810 | 3.54E−03 | — | >0.05 | — | >0.05 |
| GO: 0043122 | regulation of I-kappaB kinase/NF- | 30/810 | 1.30E−06 | 30/941 | 2.46E−05 | — | >0.05 |
| | kappaB signaling | | | | | | |
| GO: 0043123 | positive regulation of I-kappaB | 24/810 | 4.23E−06 | 24/941 | 4.93E−05 | — | >0.05 |
| | kinase/NF-kappaB signaling | | | | | | |
| GO: 0043153 | entrainment of circadian clock by | 5/810 | 4.20E−03 | — | >0.05 | — | >0.05 |
| | photoperiod | | | | | | |
| GO: 0043154 | negative regulation of cysteine-type | 11/810 | 6.02E−04 | 12/941 | 5.85E−04 | — | >0.05 |
| | endopeptidase activity involved in | | | | | | |
| | apoptotic process | | | | | | |
| GO: 0043161 | proteasome-mediated ubiquitin- | 40/810 | 9.88E−06 | 39/941 | 5.12E−04 | — | >0.05 |
| | dependent protein catabolic | | | | | | |
| | process | | | | | | |
| GO: 0043162 | ubiquitin-dependent protein | 6/810 | 5.22E−03 | — | >0.05 | — | >0.05 |
| | catabolic process via the | | | | | | |
| | multivesicular body sorting pathway | | | | | | |
| GO: 0043197 | dendritic spine | — | >0.05 | 17/959 | 6.74E−03 | 29/1866 | 3.01E−03 |
| GO: 0043200 | response to amino acid | 13/810 | 2.28E−03 | — | >0.05 | — | >0.05 |
| GO: 0043204 | perikaryon | — | >0.05 | — | >0.05 | 31/1866 | 7.79E−05 |
| GO: 0043244 | regulation of protein-containing | 12/810 | 7.68E−03 | — | >0.05 | — | >0.05 |
| | complex disassembly | | | | | | |
| GO: 0043249 | erythrocyte maturation | 5/810 | 6.56E−04 | 5/941 | 1.29E−03 | — | >0.05 |
| GO: 0043254 | regulation of protein-containing | 44/810 | 4.04E−08 | 48/941 | 6.46E−08 | 63/1792 | 1.55E−04 |
| | complex assembly | | | | | | |
| GO: 0043280 | positive regulation of cysteine-type | 13/810 | 3.28E−03 | 14/941 | 4.51E−03 | — | >0.05 |
| | endopeptidase activity involved in | | | | | | |
| | apoptotic process | | | | | | |
| GO: 0043281 | regulation of cysteine-type | 22/810 | 1.03E−04 | 24/941 | 1.29E−04 | — | >0.05 |
| | endopeptidase activity involved in | | | | | | |
| | apoptotic process | | | | | | |
| GO: 0043299 | leukocyte degranulation | 12/810 | 1.91E−04 | 12/941 | 7.38E−04 | 17/1792 | 1.60E−03 |
| GO: 0043300 | regulation of leukocyte | 8/810 | 1.09E−03 | 8/941 | 2.81E−03 | — | >0.05 |
| | degranulation | | | | | | |
| GO: 0043301 | negative regulation of leukocyte | 4/810 | 2.64E−03 | 4/941 | 4.54E−03 | — | >0.05 |
| | degranulation | | | | | | |
| GO: 0043303 | mast cell degranulation | 7/810 | 8.35E−03 | — | >0.05 | — | >0.05 |
| GO: 0043388 | positive regulation of DNA binding | 9/810 | 8.13E−04 | 9/941 | 2.30E−03 | — | >0.05 |
| GO: 0043393 | regulation of protein binding | 19/810 | 1.41E−03 | 19/941 | 7.19E−03 | — | >0.05 |
| GO: 0043407 | negative regulation of MAP kinase | 11/810 | 4.64E−05 | 11/941 | 1.76E−04 | 16/1792 | 1.19E−04 |
| | activity | | | | | | |
| GO: 0043409 | negative regulation of MAPK | 22/810 | 1.64E−05 | 22/941 | 1.51E−04 | 32/1792 | 6.74E−04 |
| | cascade | | | | | | |
| GO: 0043410 | positive regulation of MAPK | 34/810 | 6.91E−03 | — | >0.05 | — | >0.05 |
| | cascade | | | | | | |
| GO: 0043434 | response to peptide hormone | 40/810 | 6.35E−06 | 45/941 | 3.78E−06 | 74/1792 | 7.04E−07 |
| GO: 0043467 | regulation of generation of | 19/810 | 7.51E−06 | 20/941 | 1.79E−05 | 25/1792 | 1.27E−03 |
| | precursor metabolites and energy | | | | | | |
| GO: 0043470 | regulation of carbohydrate catabolic | 9/810 | 8.13E−04 | 9/941 | 2.30E−03 | — | >0.05 |
| | process | | | | | | |
| GO: 0043484 | regulation of RNA splicing | 28/810 | 6.84E−09 | 29/941 | 4.46E−08 | 34/1792 | 1.44E−04 |
| GO: 0043487 | regulation of RNA stability | 22/810 | 1.19E−04 | 24/941 | 1.50E−04 | — | >0.05 |
| GO: 0043488 | regulation of mRNA stability | 22/810 | 4.84E−05 | 24/941 | 5.83E−05 | — | >0.05 |
| GO: 0043489 | RNA stabilization | 10/810 | 1.07E−03 | 12/941 | 2.39E−04 | — | >0.05 |
| GO: 0043491 | protein kinase B signaling | 21/810 | 5.32E−04 | 22/941 | 1.51E−03 | — | >0.05 |
| GO: 0043505 | CENP-A containing nucleosome | 7/820 | 3.37E−06 | 7/959 | 9.49E−06 | 7/1866 | 6.54E−04 |
| GO: 0043508 | negative regulation of JUN kinase | 5/810 | 4.80E−04 | 5/941 | 9.52E−04 | — | >0.05 |
| | activity | | | | | | |
| GO: 0043523 | regulation of neuron apoptotic | 21/810 | 6.38E−04 | 21/941 | 3.94E−03 | 38/1792 | 2.73E−04 |
| | process | | | | | | |
| GO: 0043524 | negative regulation of neuron | 15/810 | 3.19E−03 | — | >0.05 | 29/1792 | 3.66E−04 |
| | apoptotic process | | | | | | |
| GO: 0043535 | regulation of blood vessel | 14/810 | 7.42E−03 | 16/941 | 5.00E−03 | — | >0.05 |
| | endothelial cell migration | | | | | | |
| GO: 0043536 | positive regulation of blood vessel | 12/810 | 1.01E−04 | 14/941 | 2.36E−05 | — | >0.05 |
| | endothelial cell migration | | | | | | |
| GO: 0043542 | endothelial cell migration | 24/810 | 1.75E−03 | 27/941 | 1.45E−03 | — | >0.05 |
| GO: 0043555 | regulation of translation in response | 7/810 | 3.02E−05 | 7/941 | 7.87E−05 | 8/1792 | 7.41E−04 |
| | to stress | | | | | | |
| GO: 0043558 | regulation of translational initiation | 4/810 | 3.48E−03 | 4/941 | 5.95E−03 | — | >0.05 |
| | in response to stress | | | | | | |
| GO: 0043618 | regulation of transcription from RNA | 8/810 | 2.09E−04 | 8/941 | 5.73E−04 | 10/1792 | 2.82E−03 |
| | polymerase II promoter in response | | | | | | |
| | to stress | | | | | | |
| GO: 0043620 | regulation of DNA-templated | 10/810 | 1.68E−05 | 10/941 | 6.02E−05 | 12/1792 | 7.81E−04 |
| | transcription in response to stress | | | | | | |
| GO: 0043903 | regulation of biological process | 9/810 | 1.34E−03 | 9/941 | 3.70E−03 | — | >0.05 |
| | involved in symbiotic interaction | | | | | | |
| GO: 0044000 | movement in host | 24/810 | 1.98E−06 | 25/941 | 8.14E−06 | — | >0.05 |
| GO: 0044091 | membrane biogenesis | 13/810 | 3.56E−06 | 13/941 | 1.79E−05 | — | >0.05 |
| GO: 0044183 | protein folding chaperone | 10/812 | 7.50E−04 | 11/949 | 6.61E−04 | — | >0.05 |
| GO: 0044270 | cellular nitrogen compound | 43/810 | 9.85E−06 | 46/941 | 3.81E−05 | — | >0.05 |
| | catabolic process | | | | | | |
| GO: 0044309 | neuron spine | — | >0.05 | 17/959 | 7.94E−03 | 29/1866 | 3.88E−03 |
| GO: 0044389 | ubiquitin-like protein ligase binding | 49/812 | 2.95E−14 | 47/949 | 1.09E−10 | 58/1815 | 4.64E−06 |
| GO: 0044391 | ribosomal subunit | 51/820 | 1.89E−27 | 51/959 | 2.58E−24 | 53/1866 | 2.46E−13 |
| GO: 0044403 | biological process involved in | 30/810 | 5.55E−05 | 32/941 | 1.54E−04 | — | >0.05 |
| | symbiotic interaction | | | | | | |
| GO: 0044548 | S100 protein binding | 4/812 | 2.78E−03 | 4/949 | 4.89E−03 | — | >0.05 |
| GO: 0044753 | amphisome | 6/820 | 1.33E−05 | 6/959 | 3.25E−05 | 6/1866 | 1.26E−03 |
| GO: 0044769 | ATPase activity, coupled to | — | >0.05 | 6/949 | 1.21E−03 | — | >0.05 |
| | transmembrane movement of ions, | | | | | | |
| | rotational mechanism | | | | | | |
| GO: 0044772 | mitotic cell cycle phase transition | 39/810 | 1.02E−04 | 43/941 | 1.45E−04 | — | >0.05 |
| GO: 0044815 | DNA packaging complex | 29/820 | 1.90E−11 | 30/959 | 1.63E−10 | 31/1866 | 8.83E−05 |
| GO: 0044843 | cell cycle G1/S phase transition | 26/810 | 2.79E−04 | 29/941 | 2.45E−04 | — | >0.05 |
| GO: 0045010 | actin nucleation | 8/810 | 3.38E−03 | 9/941 | 2.30E−03 | — | >0.05 |
| GO: 0045047 | protein targeting to ER | 7/810 | 2.55E−03 | 7/941 | 5.82E−03 | — | >0.05 |
| GO: 0045055 | regulated exocytosis | 19/810 | 5.77E−03 | 21/941 | 6.59E−03 | 39/1792 | 3.41E−04 |
| GO: 0045059 | positive thymic T cell selection | — | >0.05 | 4/941 | 4.54E−03 | — | >0.05 |
| GO: 0045061 | thymic T cell selection | — | >0.05 | 5/941 | 4.44E−03 | — | >0.05 |
| GO: 0045069 | regulation of viral genome | 14/810 | 2.24E−05 | 15/941 | 2.83E−05 | — | >0.05 |
| | replication | | | | | | |
| GO: 0045070 | positive regulation of viral genome | 7/810 | 4.06E−04 | 7/941 | 9.91E−04 | — | >0.05 |
| | replication | | | | | | |
| GO: 0045071 | negative regulation of viral genome | — | >0.05 | 8/941 | 7.39E−03 | — | >0.05 |
| | replication | | | | | | |
| GO: 0045088 | regulation of innate immune | 48/810 | 2.19E−11 | 49/941 | 1.14E−09 | 59/1792 | 1.18E−04 |
| | response | | | | | | |
| GO: 0045089 | positive regulation of innate | 36/810 | 4.36E−08 | 37/941 | 5.93E−07 | 46/1792 | 1.20E−03 |
| | immune response | | | | | | |
| GO: 0045116 | protein neddylation | 6/810 | 1.19E−03 | 6/941 | 2.55E−03 | — | >0.05 |
| GO: 0045121 | membrane raft | 35/820 | 4.46E−07 | 38/959 | 8.70E−07 | 56/1866 | 1.35E−05 |
| GO: 0045165 | cell fate commitment | — | >0.05 | — | >0.05 | 51/1792 | 9.94E−06 |
| GO: 0045211 | postsynaptic membrane | — | >0.05 | — | >0.05 | 46/1866 | 2.16E−04 |
| GO: 0045259 | proton-transporting ATP synthase | 6/820 | 7.11E−05 | 7/959 | 1.49E−05 | 7/1866 | 9.81E−04 |
| | complex | | | | | | |
| GO: 0045275 | respiratory chain complex III | 4/820 | 1.24E−03 | 4/959 | 2.22E−03 | — | >0.05 |
| GO: 0045296 | cadherin binding | 69/812 | 2.70E−27 | 68/949 | 1.24E−22 | 80/1815 | 5.94E−14 |
| GO: 0045309 | protein phosphorylated amino acid | 9/812 | 1.03E−03 | 9/949 | 2.99E−03 | — | >0.05 |
| | binding | | | | | | |
| GO: 0045324 | late endosome to vacuole transport | 6/810 | 5.22E−03 | — | >0.05 | — | >0.05 |
| GO: 0045333 | cellular respiration | 28/810 | 9.82E−07 | 29/941 | 5.99E−06 | — | >0.05 |
| GO: 0045334 | clathrin-coated endocytic vesicle | 9/820 | 1.56E−02 | 10/959 | 1.53E−02 | — | >0.05 |
| GO: 0045335 | phagocytic vesicle | 16/820 | 3.48E−04 | 18/959 | 2.35E−04 | — | >0.05 |
| GO: 0045428 | regulation of nitric oxide | 8/810 | 5.70E−03 | — | >0.05 | — | >0.05 |
| | biosynthetic process | | | | | | |
| GO: 0045576 | mast cell activation | 9/810 | 2.90E−03 | 10/941 | 2.32E−03 | 15/1792 | 2.00E−03 |
| GO: 0045577 | regulation of B cell differentiation | 10/810 | 1.32E−06 | 10/941 | 5.08E−06 | 10/1792 | 1.12E−03 |
| GO: 0045579 | positive regulation of B cell | 6/810 | 3.96E−05 | 6/941 | 9.17E−05 | 6/1792 | 2.90E−03 |
| | differentiation | | | | | | |
| GO: 0045619 | regulation of lymphocyte | 21/810 | 4.69E−04 | 23/941 | 5.72E−04 | — | >0.05 |
| | differentiation | | | | | | |
| GO: 0045621 | positive regulation of lymphocyte | 13/810 | 4.92E−03 | 14/941 | 6.84E−03 | — | >0.05 |
| | differentiation | | | | | | |
| GO: 0045637 | regulation of myeloid cell | 26/810 | 2.44E−06 | 27/941 | 1.21E−05 | 36/1792 | 7.06E−04 |
| | differentiation | | | | | | |
| GO: 0045638 | negative regulation of myeloid cell | 11/810 | 2.60E−03 | — | >0.05 | — | >0.05 |
| | differentiation | | | | | | |
| GO: 0045639 | positive regulation of myeloid cell | 15/810 | 4.48E−05 | 16/941 | 6.75E−05 | 21/1792 | 8.54E−04 |
| | differentiation | | | | | | |
| GO: 0045646 | regulation of erythrocyte | 13/810 | 7.63E−08 | 14/941 | 5.49E−08 | 15/1792 | 2.40E−05 |
| | differentiation | | | | | | |
| GO: 0045648 | positive regulation of erythrocyte | 9/810 | 1.18E−05 | 10/941 | 5.08E−06 | 10/1792 | 1.12E−03 |
| | differentiation | | | | | | |
| GO: 0045652 | regulation of megakaryocyte | 6/810 | 5.22E−03 | — | >0.05 | — | >0.05 |
| | differentiation | | | | | | |
| GO: 0045653 | negative regulation of | 5/810 | 1.15E−03 | 5/941 | 2.23E−03 | — | >0.05 |
| | megakaryocyte differentiation | | | | | | |
| GO: 0045657 | positive regulation of monocyte | 3/810 | 8.14E−03 | — | >0.05 | — | >0.05 |
| | differentiation | | | | | | |
| GO: 0045666 | positive regulation of neuron | — | >0.05 | — | >0.05 | 19/1792 | 1.26E−03 |
| | differentiation | | | | | | |
| GO: 0045723 | positive regulation of fatty acid | 5/810 | 2.32E−03 | 5/941 | 4.44E−03 | 8/1792 | 7.41E−04 |
| | biosynthetic process | | | | | | |
| GO: 0045727 | positive regulation of translation | 17/810 | 1.62E−04 | 16/941 | 2.37E−03 | — | >0.05 |
| GO: 0045730 | respiratory burst | 7/810 | 1.42E−03 | 7/941 | 3.32E−03 | — | >0.05 |
| GO: 0045732 | positive regulation of protein | 21/810 | 3.41E−04 | 22/941 | 9.81E−04 | — | >0.05 |
| | catabolic process | | | | | | |
| GO: 0045736 | negative regulation of cyclin- | 6/810 | 2.45E−03 | 6/941 | 5.14E−03 | — | >0.05 |
| | dependent protein serine/threonine | | | | | | |
| | kinase activity | | | | | | |
| GO: 0045765 | regulation of angiogenesis | 26/810 | 6.62E−03 | 31/941 | 2.13E−03 | — | >0.05 |
| GO: 0045766 | positive regulation of angiogenesis | 16/810 | 7.97E−03 | 19/941 | 3.10E−03 | — | >0.05 |
| GO: 0045785 | positive regulation of cell adhesion | 38/810 | 3.94E−04 | 41/941 | 1.01E−03 | — | >0.05 |
| GO: 0045786 | negative regulation of cell cycle | 31/810 | 1.84E−03 | — | >0.05 | — | >0.05 |
| GO: 0045787 | positive regulation of cell cycle | 26/810 | 7.37E−03 | 31/941 | 2.43E−03 | — | >0.05 |
| GO: 0045806 | negative regulation of endocytosis | 8/810 | 3.02E−03 | 8/941 | 7.39E−03 | — | >0.05 |
| GO: 0045815 | transcription initiation-coupled | 5/810 | 5.94E−03 | — | >0.05 | — | >0.05 |
| | chromatin remodeling | | | | | | |
| GO: 0045861 | negative regulation of proteolysis | 27/810 | 1.39E−03 | — | >0.05 | — | >0.05 |
| GO: 0045862 | positive regulation of proteolysis | 38/810 | 7.65E−07 | 38/941 | 2.46E−05 | — | >0.05 |
| GO: 0045912 | negative regulation of carbohydrate | 7/810 | 6.76E−03 | — | >0.05 | — | >0.05 |
| | metabolic process | | | | | | |
| GO: 0045920 | negative regulation of exocytosis | 11/810 | 3.44E−07 | 11/941 | 1.51E−06 | 12/1792 | 1.31E−04 |
| GO: 0045921 | positive regulation of exocytosis | 9/810 | 8.55E−03 | — | >0.05 | — | >0.05 |
| GO: 0045926 | negative regulation of growth | 20/810 | 8.00E−03 | 23/941 | 5.17E−03 | — | >0.05 |
| GO: 0045927 | positive regulation of growth | — | >0.05 | 23/941 | 6.84E−03 | — | >0.05 |
| GO: 0045930 | negative regulation of mitotic cell | 20/810 | 5.41E−03 | — | >0.05 | — | >0.05 |
| | cycle | | | | | | |
| GO: 0045931 | positive regulation of mitotic cell | 17/810 | 5.29E−05 | 18/941 | 1.03E−04 | — | >0.05 |
| | cycle | | | | | | |
| GO: 0045936 | negative regulation of phosphate | 54/810 | 8.01E−12 | 57/941 | 8.74E−11 | 83/1792 | 2.34E−09 |
| | metabolic process | | | | | | |
| GO: 0045980 | negative regulation of nucleotide | 7/810 | 7.59E−05 | 8/941 | 2.29E−05 | 8/1792 | 1.92E−03 |
| | metabolic process | | | | | | |
| GO: 0046031 | ADP metabolic process | 15/810 | 1.47E−05 | 15/941 | 8.25E−05 | 19/1792 | 1.64E−03 |
| GO: 0046034 | ATP metabolic process | 31/810 | 2.13E−09 | 33/941 | 5.33E−09 | 39/1792 | 4.05E−05 |
| GO: 0046328 | regulation of JNK cascade | 13/810 | 8.56E−03 | — | >0.05 | — | >0.05 |
| GO: 0046329 | negative regulation of JNK cascade | 6/810 | 6.80E−03 | — | >0.05 | — | >0.05 |
| GO: 0046390 | ribose phosphate biosynthetic | 20/810 | 2.37E−03 | 21/941 | 5.97E−03 | — | >0.05 |
| | process | | | | | | |
| GO: 0046496 | nicotinamide nucleotide metabolic | 9/810 | 7.89E−03 | 10/941 | 6.95E−03 | — | >0.05 |
| | process | | | | | | |
| GO: 0046596 | regulation of viral entry into host cell | 9/810 | 2.16E−04 | 9/941 | 6.48E−04 | — | >0.05 |
| GO: 0046598 | positive regulation of viral entry into | 5/810 | 2.37E−04 | 5/941 | 4.75E−04 | — | >0.05 |
| | host cell | | | | | | |
| GO: 0046605 | regulation of centrosome cycle | 8/810 | 2.39E−03 | 8/941 | 5.92E−03 | — | >0.05 |
| GO: 0046626 | regulation of insulin receptor | 11/810 | 2.97E−04 | 11/941 | 1.04E−03 | — | >0.05 |
| | signaling pathway | | | | | | |
| GO: 0046628 | positive regulation of insulin | 7/810 | 7.59E−05 | 7/941 | 1.94E−04 | 8/1792 | 1.92E−03 |
| | receptor signaling pathway | | | | | | |
| GO: 0046651 | lymphocyte proliferation | 34/810 | 6.97E−07 | 36/941 | 2.79E−06 | 47/1792 | 1.11E−03 |
| GO: 0046685 | response to arsenic-containing | 6/810 | 2.45E−03 | 6/941 | 5.14E−03 | — | >0.05 |
| | substance | | | | | | |
| GO: 0046697 | decidualization | 5/810 | 4.20E−03 | — | >0.05 | — | >0.05 |
| GO: 0046700 | heterocycle catabolic process | 44/810 | 4.81E−06 | 47/941 | 2.03E−05 | — | >0.05 |
| GO: 0046718 | viral entry into host cell | 14/810 | 7.42E−03 | — | >0.05 | — | >0.05 |
| GO: 0046753 | non-lytic viral release | 6/810 | 2.57E−05 | 6/941 | 5.99E−05 | 6/1792 | 1.97E−03 |
| GO: 0046755 | viral budding | 6/810 | 9.67E−04 | 6/941 | 2.09E−03 | — | >0.05 |
| GO: 0046761 | viral budding from plasma | 6/810 | 1.60E−05 | 6/941 | 3.76E−05 | 6/1792 | 1.29E−03 |
| | membrane | | | | | | |
| GO: 0046822 | regulation of nucleocytoplasmic | 14/810 | 2.55E−04 | 16/941 | 1.08E−04 | — | >0.05 |
| | transport | | | | | | |
| GO: 0046824 | positive regulation of | — | >0.05 | 9/941 | 3.30E−03 | — | >0.05 |
| | nucleocytoplasmic transport | | | | | | |
| GO: 0046825 | regulation of protein export from | 6/810 | 2.45E−03 | 6/941 | 5.14E−03 | — | >0.05 |
| | nucleus | | | | | | |
| GO: 0046879 | hormone secretion | — | >0.05 | 26/941 | 7.54E−03 | — | >0.05 |
| GO: 0046889 | positive regulation of lipid | 10/810 | 4.92E−03 | — | >0.05 | — | >0.05 |
| | biosynthetic process | | | | | | |
| GO: 0046933 | proton-transporting ATP synthase | 7/812 | 2.82E−06 | 8/949 | 4.82E−07 | 8/1815 | 6.10E−05 |
| | activity, rotational mechanism | | | | | | |
| GO: 0046939 | nucleotide phosphorylation | 15/810 | 5.03E−05 | 15/941 | 2.63E−04 | 20/1792 | 2.35E−03 |
| GO: 0046961 | proton-transporting ATPase activity, | — | >0.05 | 6/949 | 1.21E−03 | — | >0.05 |
| | rotational mechanism | | | | | | |
| GO: 0046982 | protein heterodimerization activity | 41/812 | 4.35E−09 | 47/949 | 5.28E−10 | 63/1815 | 5.00E−07 |
| GO: 0047497 | mitochondrion transport along | 5/810 | 5.94E−03 | — | >0.05 | — | >0.05 |
| | microtubule | | | | | | |
| GO: 0048010 | vascular endothelial growth factor | 8/810 | 3.77E−03 | 9/941 | 2.60E−03 | — | >0.05 |
| | receptor signaling pathway | | | | | | |
| GO: 0048015 | phosphatidylinositol-mediated | — | >0.05 | 17/941 | 7.28E−03 | — | >0.05 |
| | signaling | | | | | | |
| GO: 0048024 | regulation of mRNA splicing, via | 18/810 | 9.57E−07 | 18/941 | 7.83E−06 | 22/1792 | 5.77E−04 |
| | spliceosome | | | | | | |
| GO: 0048025 | negative regulation of mRNA | 6/810 | 2.94E−04 | 6/941 | 6.56E−04 | — | >0.05 |
| | splicing, via spliceosome | | | | | | |
| GO: 0048048 | embryonic eye morphogenesis | — | >0.05 | — | >0.05 | 10/1792 | 1.43E−03 |
| GO: 0048255 | mRNA stabilization | 10/810 | 3.54E−04 | 12/941 | 6.17E−05 | 14/1792 | 2.21E−03 |
| GO: 0048259 | regulation of receptor-mediated | — | >0.05 | 13/941 | 6.02E−03 | — | >0.05 |
| | endocytosis | | | | | | |
| GO: 0048261 | negative regulation of receptor- | 6/810 | 4.54E−03 | — | >0.05 | — | >0.05 |
| | mediated endocytosis | | | | | | |
| GO: 0048284 | organelle fusion | 16/810 | 1.48E−03 | 16/941 | 6.41E−03 | — | >0.05 |
| GO: 0048385 | regulation of retinoic acid receptor | — | >0.05 | — | >0.05 | 6/1792 | 2.90E−03 |
| | signaling pathway | | | | | | |
| GO: 0048475 | coated membrane | 12/820 | 5.34E−04 | 16/959 | 1.17E−05 | 20/1866 | 4.51E−04 |
| GO: 0048500 | signal recognition particle | 5/820 | 5.23E−05 | 5/959 | 1.10E−04 | 5/1866 | 2.41E−03 |
| GO: 0048524 | positive regulation of viral process | 13/810 | 3.56E−06 | 13/941 | 1.79E−05 | — | >0.05 |
| GO: 0048525 | negative regulation of viral process | 11/810 | 2.00E−03 | 12/941 | 2.10E−03 | — | >0.05 |
| GO: 0048534 | hematopoietic or lymphoid organ | — | >0.05 | — | >0.05 | 19/1792 | 2.12E−03 |
| | development | | | | | | |
| GO: 0048545 | response to steroid hormone | 29/810 | 4.41E−04 | 31/941 | 1.11E−03 | 49/1792 | 2.68E−03 |
| GO: 0048562 | embryonic organ morphogenesis | — | >0.05 | — | >0.05 | 48/1792 | 2.64E−04 |
| GO: 0048568 | embryonic organ development | — | >0.05 | — | >0.05 | 74/1792 | 6.58E−06 |
| GO: 0048592 | eye morphogenesis | — | >0.05 | — | >0.05 | 29/1792 | 5.70E−04 |
| GO: 0048593 | camera-type eye morphogenesis | — | >0.05 | — | >0.05 | 25/1792 | 5.04E−04 |
| GO: 0048596 | embryonic camera-type eye | — | >0.05 | — | >0.05 | 9/1792 | 5.28E−04 |
| | morphogenesis | | | | | | |
| GO: 0048638 | regulation of developmental growth | — | >0.05 | 28/941 | 5.85E−03 | — | >0.05 |
| GO: 0048641 | regulation of skeletal muscle tissue | 5/810 | 5.94E−03 | 6/941 | 2.09E−03 | — | >0.05 |
| | development | | | | | | |
| GO: 0048643 | positive regulation of skeletal | — | >0.05 | 5/941 | 2.85E−03 | 7/1792 | 2.06E−03 |
| | muscle tissue development | | | | | | |
| GO: 0048662 | negative regulation of smooth | 10/810 | 1.63E−03 | 10/941 | 4.80E−03 | — | >0.05 |
| | muscle cell proliferation | | | | | | |
| GO: 0048663 | neuron fate commitment | — | >0.05 | — | >0.05 | 19/1792 | 1.78E−05 |
| GO: 0048665 | neuron fate specification | — | >0.05 | — | >0.05 | 10/1792 | 8.66E−04 |
| GO: 0048704 | embryonic skeletal system morphogenesis | — | >0.05 | — | >0.05 | 21/1792 | 2.73E−04 |
| GO: 0048705 | skeletal system morphogenesis | — | >0.05 | — | >0.05 | 40/1792 | 1.67E−04 |
| GO: 0048706 | embryonic skeletal system | — | >0.05 | — | >0.05 | 28/1792 | 4.05E−05 |
| | development | | | | | | |
| GO: 0048708 | astrocyte differentiation | — | >0.05 | 11/941 | 4.80E−03 | — | >0.05 |
| GO: 0048732 | gland development | — | >0.05 | 40/941 | 3.30E−04 | 68/1792 | 9.12E−05 |
| GO: 0048770 | pigment granule | 21/820 | 9.74E−09 | 21/959 | 1.41E−07 | 23/1866 | 4.19E−04 |
| GO: 0048821 | erythrocyte development | 12/810 | 6.53E−08 | 12/941 | 3.32E−07 | 12/1792 | 2.29E−04 |
| GO: 0048872 | homeostasis of number of cells | 50/810 | 4.73E−16 | 53/941 | 2.55E−15 | 58/1792 | 3.75E−07 |
| GO: 0048880 | sensory system development | — | >0.05 | — | >0.05 | 65/1792 | 1.86E−05 |
| GO: 0050000 | chromosome localization | 10/810 | 7.28E−03 | — | >0.05 | — | >0.05 |
| GO: 0050657 | nucleic acid transport | 15/810 | 4.58E−03 | — | >0.05 | — | >0.05 |
| GO: 0050658 | RNA transport | 15/810 | 4.58E−03 | — | >0.05 | — | >0.05 |
| GO: 0050670 | regulation of lymphocyte | 28/810 | 2.32E−06 | 30/941 | 5.00E−06 | 40/1792 | 4.57E−04 |
| | proliferation | | | | | | |
| GO: 0050671 | positive regulation of lymphocyte | 16/810 | 6.27E−04 | 17/941 | 1.14E−03 | — | >0.05 |
| | proliferation | | | | | | |
| GO: 0050672 | negative regulation of lymphocyte | 11/810 | 1.66E−03 | 12/941 | 1.73E−03 | 19/1792 | 7.13E−04 |
| | proliferation | | | | | | |
| GO: 0050681 | nuclear androgen receptor binding | 5/812 | 5.33E−03 | — | >0.05 | — | >0.05 |
| GO: 0050684 | regulation of mRNA processing | 20/810 | 2.75E−06 | 20/941 | 2.49E−05 | 25/1792 | 1.75E−03 |
| GO: 0050686 | negative regulation of mRNA | 6/810 | 4.92E−04 | 6/941 | 1.08E−03 | — | >0.05 |
| | processing | | | | | | |
| GO: 0050687 | negative regulation of defense | 9/810 | 1.95E−05 | 9/941 | 6.32E−05 | — | >0.05 |
| | response to virus | | | | | | |
| GO: 0050688 | regulation of defense response to | 11/810 | 6.02E−04 | 11/941 | 2.02E−03 | — | >0.05 |
| | virus | | | | | | |
| GO: 0050727 | regulation of inflammatory response | — | >0.05 | 33/941 | 7.64E−03 | — | >0.05 |
| GO: 0050729 | positive regulation of inflammatory | 14/810 | 8.30E−03 | — | >0.05 | — | >0.05 |
| | response | | | | | | |
| GO: 0050750 | low-density lipoprotein particle | 5/812 | 3.71E−03 | — | >0.05 | — | >0.05 |
| | receptor binding | | | | | | |
| GO: 0050777 | negative regulation of immune | 21/810 | 1.11E−04 | 22/941 | 3.27E−04 | — | >0.05 |
| | response | | | | | | |
| GO: 0050780 | dopamine receptor binding | 5/812 | 5.14E−04 | 6/949 | 1.03E−04 | — | >0.05 |
| GO: 0050792 | regulation of viral process | 24/810 | 2.65E−07 | 25/941 | 1.10E−06 | — | >0.05 |
| GO: 0050803 | regulation of synapse structure or | — | >0.05 | 22/941 | 5.84E−03 | 43/1792 | 5.92E−05 |
| | activity | | | | | | |
| GO: 0050804 | modulation of chemical synaptic | — | >0.05 | — | >0.05 | 69/1792 | 1.01E−03 |
| | transmission | | | | | | |
| GO: 0050806 | positive regulation of synaptic | — | >0.05 | — | >0.05 | 33/1792 | 9.31E−05 |
| | transmission | | | | | | |
| GO: 0050807 | regulation of synapse organization | — | >0.05 | 22/941 | 4.33E−03 | 42/1792 | 6.86E−05 |
| GO: 0050808 | synapse organization | — | >0.05 | — | >0.05 | 74/1792 | 1.67E−05 |
| GO: 0050821 | protein stabilization | 28/810 | 1.98E−07 | 28/941 | 3.79E−06 | 35/1792 | 1.27E−03 |
| GO: 0050851 | antigen receptor-mediated signaling | 26/810 | 8.01E−07 | 26/941 | 1.21E−05 | — | >0.05 |
| | pathway | | | | | | |
| GO: 0050852 | cell receptor signaling pathway | 17/810 | 1.36E−04 | 17/941 | 7.64E−04 | — | >0.05 |
| GO: 0050853 | cell receptor signaling pathway | 12/810 | 1.32E−04 | 12/941 | 5.18E−04 | — | >0.05 |
| GO: 0050860 | negative regulation of T cell | 5/810 | 4.20E−03 | — | >0.05 | — | >0.05 |
| | receptor signaling pathway | | | | | | |
| GO: 0050863 | regulation of T cell activation | 33/810 | 1.42E−04 | 35/941 | 4.96E−04 | — | >0.05 |
| GO: 0050864 | regulation of B cell activation | 16/810 | 1.96E−04 | 17/941 | 3.50E−04 | — | >0.05 |
| GO: 0050866 | negative regulation of | 25/810 | 2.15E−05 | 26/941 | 9.30E−05 | — | >0.05 |
| | cell activation | | | | | | |
| GO: 0050867 | positive regulation of cell activation | 36/810 | 2.95E−05 | 38/941 | 1.35E−04 | — | >0.05 |
| GO: 0050868 | negative regulation of T cell | 13/810 | 5.25E−03 | 14/941 | 7.31E−03 | — | >0.05 |
| | activation | | | | | | |
| GO: 0050869 | negative regulation of B cell | 6/810 | 3.38E−03 | 6/941 | 7.00E−03 | — | >0.05 |
| | activation | | | | | | |
| GO: 0050870 | positive regulation of T cell | 25/810 | 1.07E−04 | 26/941 | 4.40E−04 | — | >0.05 |
| | activation | | | | | | |
| GO: 0050878 | regulation of body fluid levels | — | >0.05 | 30/941 | 6.23E−03 | — | >0.05 |
| GO: 0050900 | leukocyte migration | 35/810 | 5.77E−05 | 38/941 | 1.15E−04 | — | >0.05 |
| GO: 0051015 | actin filament binding | 31/812 | 1.84E−09 | 30/949 | 2.32E−07 | 42/1815 | 3.42E−06 |
| GO: 0051018 | protein kinase A binding | — | >0.05 | 8/949 | 4.69E−03 | — | >0.05 |
| GO: 0051020 | GTPase binding | 35/812 | 2.86E−07 | 35/949 | 9.63E−06 | 49/1815 | 5.08E−04 |
| GO: 0051047 | positive regulation of secretion | 29/810 | 1.75E−04 | 33/941 | 9.18E−05 | 52/1792 | 1.33E−04 |
| GO: 0051048 | negative regulation of secretion | 17/810 | 2.61E−03 | 18/941 | 5.20E−03 | 33/1792 | 2.85E−04 |
| GO: 0051051 | negative regulation of transport | 43/810 | 9.85E−06 | 47/941 | 1.83E−05 | 73/1792 | 9.85E−05 |
| GO: 0051056 | regulation of small GTPase | 24/810 | 3.43E−03 | — | >0.05 | — | >0.05 |
| | mediated signal transduction | | | | | | |
| GO: 0051081 | nuclear membrane disassembly | 3/810 | 8.14E−03 | — | >0.05 | — | >0.05 |
| GO: 0051084 | ‘de novo’ post-translational protein | 6/810 | 5.97E−03 | — | >0.05 | — | >0.05 |
| | folding | | | | | | |
| GO: 0051090 | regulation of DNA-binding | 43/810 | 3.78E−06 | 45/941 | 3.13E−05 | 73/1792 | 2.81E−05 |
| | transcription factor activity | | | | | | |
| GO: 0051091 | positive regulation of DNA-binding | 31/810 | 1.14E−06 | 32/941 | 9.02E−06 | 49/1792 | 1.48E−05 |
| | transcription factor activity | | | | | | |
| GO: 0051092 | positive regulation of NF-kappaB | 22/810 | 1.96E−06 | 21/941 | 6.43E−05 | — | >0.05 |
| | transcription factor activity | | | | | | |
| GO: 0051098 | regulation of binding | 34/810 | 3.89E−05 | 34/941 | 6.44E−04 | — | >0.05 |
| GO: 0051099 | positive regulation of binding | 23/810 | 2.83E−06 | 23/941 | 3.18E−05 | — | >0.05 |
| GO: 0051101 | regulation of DNA binding | 15/810 | 2.59E−04 | 15/941 | 1.22E−03 | — | >0.05 |
| GO: 0051131 | chaperone-mediated protein | 6/810 | 3.83E−04 | 6/941 | 8.49E−04 | — | >0.05 |
| | complex assembly | | | | | | |
| GO: 0051156 | glucose 6-phosphate metabolic | 7/810 | 1.30E−04 | 7/941 | 3.27E−04 | 9/1792 | 7.24E−04 |
| | process | | | | | | |
| GO: 0051168 | nuclear export | 18/810 | 3.98E−04 | 18/941 | 2.18E−03 | — | >0.05 |
| GO: 0051169 | nuclear transport | 36/810 | 4.81E−07 | 38/941 | 2.33E−06 | — | >0.05 |
| GO: 0051170 | import into nucleus | 18/810 | 4.60E−04 | 20/941 | 3.91E−04 | — | >0.05 |
| GO: 0051219 | phosphoprotein binding | 14/812 | 5.69E−05 | 14/949 | 2.92E−04 | — | >0.05 |
| GO: 0051222 | positive regulation of protein | 28/810 | 2.50E−04 | 31/941 | 2.70E−04 | — | >0.05 |
| | transport | | | | | | |
| GO: 0051225 | spindle assembly | 15/810 | 6.69E−04 | 17/941 | 3.83E−04 | — | >0.05 |
| GO: 0051236 | establishment of RNA localization | 15/810 | 5.44E−03 | — | >0.05 | — | >0.05 |
| GO: 0051250 | negative regulation of lymphocyte | 21/810 | 1.66E−05 | 22/941 | 4.95E−05 | 29/1792 | 1.72E−03 |
| | activation | | | | | | |
| GO: 0051251 | positive regulation of lymphocyte | 32/810 | 2.11E−05 | 34/941 | 6.96E−05 | — | >0.05 |
| | activation | | | | | | |
| GO: 0051258 | protein polymerization | 36/810 | 5.25E−09 | 38/941 | 2.50E−08 | 45/1792 | 3.84E−04 |
| GO: 0051287 | NAD binding | 8/812 | 2.04E−03 | 8/949 | 5.29E−03 | — | >0.05 |
| GO: 0051298 | centrosome duplication | 9/810 | 5.14E−03 | — | >0.05 | — | >0.05 |
| GO: 0051310 | metaphase plate congression | 9/810 | 4.28E−03 | — | >0.05 | — | >0.05 |
| GO: 0051348 | negative regulation of transferase | 42/810 | 2.58E−12 | 43/941 | 8.28E−11 | 56/1792 | 1.15E−07 |
| | activity | | | | | | |
| GO: 0051402 | neuron apoptotic process | 24/810 | 4.32E−04 | 24/941 | 3.26E−03 | 45/1792 | 7.18E−05 |
| GO: 0051403 | stress-activated MAPK cascade | 25/810 | 5.14E−05 | 25/941 | 5.13E−04 | — | >0.05 |
| GO: 0051409 | response to nitrosative stress | 3/810 | 8.14E−03 | — | >0.05 | — | >0.05 |
| GO: 0051438 | regulation of ubiquitin-protein | 11/810 | 3.93E−05 | 11/941 | 1.50E−04 | — | >0.05 |
| | transferase activity | | | | | | |
| GO: 0051444 | negative regulation of ubiquitin- | 7/810 | 3.02E−05 | 7/941 | 7.87E−05 | — | >0.05 |
| | protein transferase activity | | | | | | |
| GO: 0051469 | vesicle fusion with vacuole | 5/810 | 6.11E−05 | 5/941 | 1.25E−04 | 5/1792 | 2.46E−03 |
| GO: 0051494 | negative regulation of cytoskeleton | 19/810 | 1.08E−04 | 19/941 | 7.02E−04 | — | >0.05 |
| | organization | | | | | | |
| GO: 0051607 | defense response to virus | 31/810 | 1.02E−05 | 31/941 | 1.69E−04 | — | >0.05 |
| GO: 0051648 | vesicle localization | | >0.05 | 21/941 | 3.54E−03 | — | >0.05 |
| GO: 0051656 | establishment of organelle | 40/810 | 1.60E−05 | 44/941 | 2.27E−05 | — | >0.05 |
| | localization | | | | | | |
| GO: 0051693 | actin filament capping | 6/810 | 7.71E−03 | — | >0.05 | — | >0.05 |
| GO: 0051701 | biological process involved in | 26/810 | 1.29E−06 | 27/941 | 6.48E−06 | — | >0.05 |
| | interaction with host | | | | | | |
| GO: 0051896 | regulation of protein kinase B | 19/810 | 5.51E−04 | 19/941 | 3.10E−03 | — | >0.05 |
| | signaling | | | | | | |
| GO: 0051897 | positive regulation of protein kinase | 14/810 | 6.43E−04 | 14/941 | 2.65E−03 | — | >0.05 |
| | B signaling | | | | | | |
| GO: 0051972 | regulation of telomerase activity | 10/810 | 3.10E−05 | 10/941 | 1.09E−04 | — | >0.05 |
| GO: 0051973 | positive regulation of telomerase | 7/810 | 6.01E−04 | 7/941 | 1.45E−03 | — | >0.05 |
| | activity | | | | | | |
| GO: 0052372 | modulation by symbiont of entry | 9/810 | 6.22E−04 | 9/941 | 1.78E−03 | — | >0.05 |
| | into host | | | | | | |
| GO: 0052547 | regulation of peptidase activity | 33/810 | 1.10E−03 | 34/941 | 6.28E−03 | — | >0.05 |
| GO: 0052548 | regulation of endopeptidase activity | 30/810 | 2.09E−04 | 31/941 | 1.17E−03 | — | >0.05 |
| GO: 0055038 | recycling endosome membrane | 10/820 | 1.11E−02 | 11/959 | 1.20E−02 | — | >0.05 |
| GO: 0055057 | neuroblast division | — | >0.05 | 4/941 | 4.54E−03 | 6/1792 | 1.29E−03 |
| GO: 0055072 | iron ion homeostasis | 10/810 | 5.78E−03 | 11/941 | 5.71E−03 | — | >0.05 |
| GO: 0055106 | ubiquitin-protein transferase | 7/812 | 4.59E−05 | 7/949 | 1.23E−04 | — | >0.05 |
| | regulator activity | | | | | | |
| GO: 0060142 | regulation of syncytium formation | — | >0.05 | 7/941 | 6.56E−04 | — | >0.05 |
| | by plasma membrane fusion | | | | | | |
| GO: 0060143 | positive regulation of syncytium | — | >0.05 | 6/941 | 1.08E−03 | — | >0.05 |
| | formation by plasma membrane | | | | | | |
| | fusion | | | | | | |
| GO: 0060147 | regulation of post-transcriptional | — | >0.05 | — | >0.05 | 10/1792 | 1.81E−03 |
| | gene silencing | | | | | | |
| GO: 0060205 | cytoplasmic vesicle lumen | 49/820 | 1.20E−14 | 51/959 | 3.09E−13 | 59/1866 | 1.80E−06 |
| GO: 0060236 | regulation of mitotic spindle | 8/810 | 5.10E−04 | 8/941 | 1.35E−03 | — | >0.05 |
| | organization | | | | | | |
| GO: 0060272 | embryonic skeletal joint | — | >0.05 | — | >0.05 | 5/1792 | 2.46E−03 |
| | morphogenesis | | | | | | |
| GO: 0060326 | cell chemotaxis | 28/810 | 2.78E−04 | 30/941 | 6.49E−04 | — | >0.05 |
| GO: 0060333 | type II interferon-mediated signaling | 6/810 | 6.24E−04 | 6/941 | 1.36E−03 | 8/1792 | 1.92E−03 |
| | pathway | | | | | | |
| GO: 0060337 | type I interferon-mediated signaling | 9/810 | 6.13E−03 | — | >0.05 | — | >0.05 |
| | pathway | | | | | | |
| GO: 0060348 | bone development | — | >0.05 | — | >0.05 | 37/1792 | 2.09E−03 |
| GO: 0060396 | growth hormone receptor signaling | 5/810 | 2.86E−03 | 5/941 | 5.44E−03 | — | >0.05 |
| | pathway | | | | | | |
| GO: 0060416 | response to growth hormone | 6/810 | 3.93E−03 | — | >0.05 | — | >0.05 |
| GO: 0060433 | bronchus development | — | >0.05 | — | >0.05 | 5/1792 | 2.46E−03 |
| GO: 0060438 | trachea development | — | >0.05 | — | >0.05 | 7/1792 | 1.46E−03 |
| GO: 0060439 | trachea morphogenesis | — | >0.05 | — | >0.05 | 5/1792 | 2.46E−03 |
| GO: 0060491 | regulation of cell projection | 19/810 | 1.04E−03 | 19/941 | 5.51E−03 | — | >0.05 |
| | assembly | | | | | | |
| GO: 0060538 | skeletal muscle organ development | — | >0.05 | 20/941 | 6.63E−04 | 31/1792 | 7.84E−04 |
| GO: 0060544 | regulation of necroptotic process | — | >0.05 | 6/941 | 5.14E−03 | — | >0.05 |
| GO: 0060589 | nucleosidr-triphosphatase regulator | 36/812 | 2.90E−03 | — | >0.05 | — | >0.05 |
| | activity | | | | | | |
| GO: 0060736 | prostate gland growth | — | >0.05 | — | >0.05 | 5/1792 | 2.46E−03 |
| GO: 0060742 | epithelial cell differentiation involved | — | >0.05 | — | >0.05 | 5/1792 | 2.46E−03 |
| | in prostate gland development | | | | | | |
| GO: 0060759 | regulation of response to cytokine | 21/810 | 3.36E−05 | 21/941 | 2.74E−04 | — | >0.05 |
| | stimulus | | | | | | |
| GO: 0060760 | positive regulation of response to | 8/810 | 7.58E−03 | — | >0.05 | — | >0.05 |
| | cytokine stimulus | | | | | | |
| GO: 0061013 | regulation of mRNA catabolic | 23/810 | 6.11E−05 | 25/941 | 8.41E−05 | — | >0.05 |
| | process | | | | | | |
| GO: 0061077 | chaperone-mediated protein folding | 10/810 | 8.49E−04 | 10/941 | 2.59E−03 | — | >0.05 |
| GO: 0061082 | myeloid leukocyte cytokine | 7/810 | 6.06E−03 | — | >0.05 | — | >0.05 |
| | production | | | | | | |
| GO: 0061136 | regulation of proteasomal protein | 25/810 | 1.26E−06 | 24/941 | 4.93E−05 | — | >0.05 |
| | catabolic process | | | | | | |
| GO: 0061351 | neural precursor cell proliferation | — | >0.05 | — | >0.05 | 29/1792 | 2.59E−04 |
| GO: 0061418 | regulation of transcription from RNA | 4/810 | 9.68E−04 | 4/941 | 1.69E−03 | 5/1792 | 2.46E−03 |
| | polymerase II promoter in response | | | | | | |
| | to hypoxia | | | | | | |
| GO: 0061515 | myeloid cell development | 21/810 | 6.28E−11 | 21/941 | 9.71E−10 | 22/1792 | 1.25E−05 |
| GO: 0061564 | axon development | — | >0.05 | — | >0.05 | 66/1792 | 2.58E−03 |
| GO: 0061614 | miRNA transcription | 11/810 | 2.02E−04 | 12/941 | 1.81E−04 | 17/1792 | 2.64E−04 |
| GO: 0061615 | glycolytic process through fructose- | 4/810 | 8.58E−03 | — | >0.05 | — | >0.05 |
| | 6-phosphate | | | | | | |
| GO: 0061629 | RNA polymerase II-specific DNA- | 42/812 | 4.70E−09 | 47/949 | 2.09E−09 | 63/1815 | 2.15E−06 |
| | binding transcription factor binding | | | | | | |
| GO: 0061638 | CENP-A containing chromatin | 7/820 | 3.37E−06 | 7/959 | 9.49E−06 | 7/1866 | 6.54E−04 |
| GO: 0061640 | cytoskeleton-dependent cytokinesis | 16/810 | 3.59E−05 | 17/941 | 6.13E−05 | 23/1792 | 5.09E−04 |
| GO: 0061644 | protein localization to CENP-A | 7/810 | 4.18E−06 | 7/941 | 1.12E−05 | 7/1792 | 6.72E−04 |
| | containing chromatin | | | | | | |
| GO: 0061726 | mitochondrion disassembly | 12/810 | 5.24E−04 | 12/941 | 1.91E−03 | — | >0.05 |
| GO: 0061763 | multivesicular body-lysosome | 5/810 | 6.11E−05 | 5/941 | 1.25E−04 | 5/1792 | 2.46E−03 |
| | fusion | | | | | | |
| GO: 0061844 | antimicrobial humoral immune | 11/810 | 8.33E−04 | 11/941 | 2.74E−03 | — | >0.05 |
| | response mediated by antimicrobial | | | | | | |
| | peptide | | | | | | |
| GO: 0061952 | midbody abscission | 6/810 | 8.52E−05 | 6/941 | 1.95E−04 | — | >0.05 |
| GO: 0062012 | regulation of small molecule | 29/810 | 3.12E−04 | 33/941 | 1.75E−04 | — | >0.05 |
| | metabolic process | | | | | | |
| GO: 0062098 | regulation of programmed necrotic | 6/810 | 3.93E−03 | 7/941 | 1.73E−03 | — | >0.05 |
| | cell death | | | | | | |
| GO: 0062197 | cellular response to chemical stress | 40/810 | 3.16E−08 | 42/941 | 2.29E−07 | — | >0.05 |
| GO: 0062207 | regulation of pattern recognition | 15/810 | 7.03E−05 | 16/941 | 1.08E−04 | — | >0.05 |
| | receptor signaling pathway | | | | | | |
| GO: 0062208 | positive regulation of pattern | 8/810 | 7.01E−04 | 8/941 | 1.84E−03 | — | >0.05 |
| | recognition receptor signaling | | | | | | |
| | pathway | | | | | | |
| GO: 0065004 | protein-DNA complex assembly | 26/810 | 2.31E−05 | 30/941 | 5.92E−06 | — | >0.05 |
| GO: 0070069 | cytochrome complex | 6/820 | 5.11E−03 | 6/959 | 1.07E−02 | — | >0.05 |
| GO: 0070167 | regulation of biomineral tissue | — | >0.05 | — | >0.05 | 20/1792 | 1.44E−03 |
| | development | | | | | | |
| GO: 0070227 | lymphocyte apoptotic process | 10/810 | 2.66E−03 | 10/941 | 7.59E−03 | — | >0.05 |
| GO: 0070228 | regulation of lymphocyte apoptotic | 9/810 | 1.05E−03 | 9/941 | 2.94E−03 | — | >0.05 |
| | process | | | | | | |
| GO: 0070232 | regulation of T cell apoptotic | 7/810 | 1.42E−03 | 7/941 | 3.32E−03 | — | >0.05 |
| | process | | | | | | |
| GO: 0070302 | regulation of stress-activated | 20/810 | 4.33E−04 | 19/941 | 5.82E−03 | — | >0.05 |
| | protein kinase signaling cascade | | | | | | |
| GO: 0070303 | negative regulation of stress- | 9/810 | 3.48E−04 | 9/941 | 1.02E−03 | — | >0.05 |
| | activated protein kinase signaling | | | | | | |
| | cascade | | | | | | |
| GO: 0070371 | ERK1 and ERK2 cascade | 31/810 | 1.07E−04 | 31/941 | 1.35E−03 | — | >0.05 |
| GO: 0070372 | regulation of ERK1 and ERK2 | 27/810 | 7.23E−04 | — | >0.05 | — | >0.05 |
| | cascade | | | | | | |
| GO: 0070373 | negative regulation of ERK1 and | 9/810 | 6.68E−03 | — | >0.05 | — | >0.05 |
| | ERK2 cascade | | | | | | |
| GO: 0070382 | exocytic vesicle | — | >0.05 | — | >0.05 | 36/1866 | 1.79E−03 |
| GO: 0070424 | regulation of nucleotide-binding | — | >0.05 | 4/941 | 3.38E−03 | — | >0.05 |
| | oligomerization domain containing | | | | | | |
| | signaling pathway | | | | | | |
| GO: 0070469 | respirasome | 9/820 | 1.46E−02 | 10/959 | 1.42E−02 | — | >0.05 |
| GO: 0070482 | response to oxygen levels | 28/810 | 6.84E−04 | 31/941 | 7.89E−04 | — | >0.05 |
| GO: 0070486 | leukocyte aggregation | 5/810 | 1.58E−04 | 5/941 | 3.19E−04 | 6/1792 | 8.04E−04 |
| GO: 0070507 | regulation of microtubule | 16/810 | 1.39E−03 | 17/941 | 2.53E−03 | — | >0.05 |
| | cytoskeleton organization | | | | | | |
| GO: 0070585 | protein localization to mitochondrion | 14/810 | 1.81E−03 | 14/941 | 6.84E−03 | — | >0.05 |
| GO: 0070661 | leukocyte proliferation | 39/810 | 7.95E−08 | 41/941 | 5.19E−07 | 52/1792 | 1.11E−03 |
| GO: 0070663 | regulation of leukocyte proliferation | 32/810 | 2.93E−07 | 34/941 | 9.43E−07 | 44/1792 | 3.80E−04 |
| GO: 0070664 | negative regulation of leukocyte | 12/810 | 1.04E−03 | 13/941 | 1.21E−03 | 20/1792 | 8.45E−04 |
| | proliferation | | | | | | |
| GO: 0070665 | positive regulation of leukocyte | 19/810 | 1.27E−04 | 20/941 | 3.08E−04 | — | >0.05 |
| | proliferation | | | | | | |
| GO: 0070820 | tertiary granule | 17/820 | 6.66E−04 | 17/959 | 3.51E−03 | — | >0.05 |
| GO: 0070849 | response to epidermal growth factor | 8/810 | 9.46E−04 | 9/941 | 5.51E−04 | 12/1792 | 1.47E−03 |
| GO: 0070878 | primary miRNA binding | 4/812 | 6.74E−04 | 4/949 | 1.21E−03 | — | >0.05 |
| GO: 0070918 | regulatory ncRNA processing | 9/810 | 3.89E−03 | — | >0.05 | — | >0.05 |
| GO: 0070993 | translation preinitiation complex | 7/820 | 5.32E−06 | 7/959 | 1.49E−05 | 8/1866 | 1.40E−04 |
| GO: 0070997 | neuron death | 34/810 | 4.58E−05 | 36/941 | 1.77E−04 | 63/1792 | 8.32E−06 |
| GO: 0071004 | U2-type prespliceosome | 4/820 | 5.03E−03 | 5/959 | 1.15E−03 | — | >0.05 |
| GO: 0071005 | U2-type precatalytic spliceosome | 8/820 | 1.17E−03 | 8/959 | 3.12E−03 | — | >0.05 |
| GO: 0071010 | prespliceosome | 4/820 | 5.03E−03 | 5/959 | 1.15E−03 | — | >0.05 |
| GO: 0071011 | precatalytic spliceosome | 8/820 | 1.72E−03 | 8/959 | 4.52E−03 | — | >0.05 |
| GO: 0071013 | catalytic step 2 spliceosome | 19/820 | 4.28E−09 | 20/959 | 9.31E−09 | 20/1866 | 2.42E−04 |
| GO: 0071168 | protein localization to chromatin | 12/810 | 2.52E−06 | 12/941 | 1.17E−05 | — | >0.05 |
| GO: 0071214 | cellular response to abiotic stimulus | 26/810 | 4.23E−03 | 30/941 | 2.40E−03 | — | >0.05 |
| GO: 0071229 | cellular response to acid chemical | 12/810 | 4.72E−04 | 13/941 | 5.24E−04 | — | >0.05 |
| GO: 0071230 | cellular response to amino acid | 10/810 | 2.42E−03 | 10/941 | 6.95E−03 | — | >0.05 |
| | stimulus | | | | | | |
| GO: 0071241 | cellular response to inorganic | 19/810 | 6.62E−03 | 22/941 | 3.71E−03 | 40/1792 | 2.22E−04 |
| | substance | | | | | | |
| GO: 0071243 | cellular response to arsenic- | 6/810 | 1.65E−04 | 6/941 | 3.72E−04 | — | >0.05 |
| | containing substance | | | | | | |
| GO: 0071248 | cellular response to metal ion | — | >0.05 | 19/941 | 6.83E−03 | 36/1792 | 2.04E−04 |
| GO: 0071277 | cellular response to calcium ion | — | >0.05 | — | >0.05 | 18/1792 | 1.63E−03 |
| GO: 0071346 | cellular response to type II | 15/810 | 1.61E−04 | 16/941 | 2.54E−04 | — | >0.05 |
| | interferon | | | | | | |
| GO: 0071356 | cellular response to tumor necrosis | 20/810 | 3.55E−03 | 22/941 | 4.33E−03 | — | >0.05 |
| | factor | | | | | | |
| GO: 0071357 | cellular response to type I interferon | 9/810 | 6.68E−03 | — | >0.05 | — | >0.05 |
| GO: 0071359 | cellular response to dsRNA | 5/810 | 1.86E−03 | 5/941 | 3.58E−03 | — | >0.05 |
| GO: 0071364 | cellular response to epidermal | 8/810 | 5.10E−04 | 9/941 | 2.74E−04 | 11/1792 | 2.25E−03 |
| | growth factor stimulus | | | | | | |
| GO: 0071375 | cellular response to peptide | 28/810 | 2.50E−04 | 32/941 | 1.21E−04 | 51/1792 | 1.21E−04 |
| | hormone stimulus | | | | | | |
| GO: 0071378 | cellular response to growth | 5/810 | 2.86E−03 | 5/941 | 5.44E−03 | — | >0.05 |
| | hormone stimulus | | | | | | |
| GO: 0071383 | cellular response to steroid | 22/810 | 1.19E−04 | 24/941 | 1.50E−04 | — | >0.05 |
| | hormone stimulus | | | | | | |
| GO: 0071384 | cellular response to corticosteroid | 9/810 | 1.69E−03 | 10/941 | 1.29E−03 | — | >0.05 |
| | stimulus | | | | | | |
| GO: 0071385 | cellular response to glucocorticoid | 8/810 | 2.11E−03 | 9/941 | 1.36E−03 | — | >0.05 |
| | stimulus | | | | | | |
| GO: 0071453 | cellular response to oxygen levels | 21/810 | 1.99E−05 | 22/941 | 5.93E−05 | 31/1792 | 4.72E−04 |
| GO: 0071456 | cellular response to hypoxia | 20/810 | 7.35E−06 | 20/941 | 6.25E−05 | 29/1792 | 1.80E−04 |
| GO: 0071459 | protein localization to chromosome, | 8/810 | 4.31E−04 | 8/941 | 1.15E−03 | — | >0.05 |
| | centromeric region | | | | | | |
| GO: 0071470 | cellular response to osmotic stress | 7/810 | 5.41E−03 | — | >0.05 | — | >0.05 |
| GO: 0071496 | cellular response to external | 27/810 | 1.21E−03 | 31/941 | 6.44E−04 | — | >0.05 |
| | stimulus | | | | | | |
| GO: 0071542 | dopaminergic neuron differentiation | — | >0.05 | — | >0.05 | 12/1792 | 4.92E−04 |
| GO: 0071621 | granulocyte chemotaxis | 17/810 | 5.84E−05 | 17/941 | 3.50E−04 | — | >0.05 |
| GO: 0071622 | regulation of granulocyte | 7/810 | 8.35E−03 | — | >0.05 | — | >0.05 |
| | chemotaxis | | | | | | |
| GO: 0071624 | positive regulation of granulocyte | 5/810 | 6.97E−03 | — | >0.05 | — | >0.05 |
| | chemotaxis | | | | | | |
| GO: 0071674 | mononuclear cell migration | 17/810 | 7.17E−03 | — | >0.05 | — | >0.05 |
| GO: 0071675 | regulation of mononuclear cell | 12/810 | 7.21E−03 | — | >0.05 | — | >0.05 |
| | migration | | | | | | |
| GO: 0071677 | positive regulation of mononuclear | 9/810 | 4.28E−03 | — | >0.05 | — | >0.05 |
| | cell migration | | | | | | |
| GO: 0071695 | anatomical structure maturation | — | >0.05 | — | >0.05 | 38/1792 | 2.80E−03 |
| GO: 0071706 | tumor necrosis factor superfamily | 19/810 | 7.16E−04 | — | >0.05 | — | >0.05 |
| | cytokine production | | | | | | |
| GO: 0071709 | membrane assembly | 12/810 | 9.96E−06 | 12/941 | 4.39E−05 | — | >0.05 |
| GO: 0071763 | nuclear membrane organization | 10/810 | 1.35E−05 | 11/941 | 7.68E−06 | 11/1792 | 2.25E−03 |
| GO: 0071824 | protein-DNA complex subunit | 28/810 | 1.27E−05 | 32/941 | 4.08E−06 | — | >0.05 |
| | organization | | | | | | |
| GO: 0071826 | ribonucleoprotein complex subunit | 34/810 | 8.17E−10 | 34/941 | 3.50E−08 | 39/1792 | 5.81E−04 |
| | organization | | | | | | |
| GO: 0071887 | leukocyte apoptotic process | 15/810 | 1.96E−04 | 15/941 | 9.39E−04 | — | >0.05 |
| GO: 0071897 | DNA biosynthetic process | 21/810 | 1.20E−04 | 21/941 | 8.76E−04 | — | >0.05 |
| GO: 0071900 | regulation of protein | 32/810 | 1.82E−04 | 32/941 | 2.24E−03 | — | >0.05 |
| | serine/threonine kinase activity | | | | | | |
| GO: 0071901 | negative regulation of protein | 19/810 | 1.62E−06 | 19/941 | 1.40E−05 | 25/1792 | 2.66E−04 |
| | serine/threonine kinase activity | | | | | | |
| GO: 0071985 | multivesicular body sorting pathway | 8/810 | 9.46E−04 | 8/941 | 2.45E−03 | — | >0.05 |
| GO: 0072044 | collecting duct development | — | >0.05 | — | >0.05 | 7/1792 | 4.31E−04 |
| GO: 0072079 | nephron tubule formation | — | >0.05 | — | >0.05 | 7/1792 | 2.06E−03 |
| GO: 0072087 | renal vesicle development | — | >0.05 | — | >0.05 | 7/1792 | 1.46E−03 |
| GO: 0072331 | signal transduction by p53 class | 21/810 | 2.38E−05 | 21/941 | 1.99E−04 | — | >0.05 |
| | mediator | | | | | | |
| GO: 0072332 | intrinsic apoptotic signaling pathway | 12/810 | 2.72E−04 | 11/941 | 3.33E−03 | — | >0.05 |
| | by p53 class mediator | | | | | | |
| GO: 0072384 | organelle transport along | 10/810 | 6.75E−03 | 11/941 | 6.75E−03 | — | >0.05 |
| | microtubule | | | | | | |
| GO: 0072498 | embryonic skeletal joint | — | >0.05 | 4/941 | 5.95E−03 | 7/1792 | 2.65E−04 |
| | development | | | | | | |
| GO: 0072522 | purine-containing compound | — | >0.05 | 23/941 | 6.84E−03 | — | >0.05 |
| | biosynthetic process | | | | | | |
| GO: 0072593 | reactive oxygen species metabolic | 29/810 | 5.21E−07 | 35/941 | 1.16E−08 | 45/1792 | 7.08E−06 |
| | process | | | | | | |
| GO: 0072594 | establishment of protein localization | 42/810 | 3.67E−06 | 44/941 | 2.82E−05 | — | >0.05 |
| | to organelle | | | | | | |
| GO: 0072599 | establishment of protein localization | 7/810 | 4.27E−03 | — | >0.05 | — | >0.05 |
| | to endoplasmic reticulum | | | | | | |
| GO: 0072655 | establishment of protein localization | 13/810 | 3.06E−03 | — | >0.05 | — | >0.05 |
| | to mitochondrion | | | | | | |
| GO: 0072659 | protein localization to plasma | 23/810 | 3.37E−03 | 28/941 | 6.14E−04 | — | >0.05 |
| | membrane | | | | | | |
| GO: 0075294 | positive regulation by symbiont of | 5/810 | 2.37E−04 | 5/941 | 4.75E−04 | — | >0.05 |
| | entry into host | | | | | | |
| GO: 0080008 | Cul4-RING E3 ubiquitin ligase | 7/820 | 7.12E−04 | 7/959 | 1.77E−03 | — | >0.05 |
| | complex | | | | | | |
| GO: 0080164 | regulation of nitric oxide metabolic | 8/810 | 7.58E−03 | — | >0.05 | — | >0.05 |
| | process | | | | | | |
| GO: 0080171 | lytic vacuole organization | 11/810 | 4.24E−03 | 12/941 | 4.67E−03 | — | >0.05 |
| GO: 0090023 | positive regulation of neutrophil | 5/810 | 4.20E−03 | — | >0.05 | — | >0.05 |
| | chemotaxis | | | | | | |
| GO: 0090066 | regulation of anatomical structure | 39/810 | 2.86E−04 | 46/941 | 5.69E−05 | — | >0.05 |
| | size | | | | | | |
| GO: 0090079 | translation regulator activity, nucleic | 19/812 | 2.91E−07 | 19/949 | 2.99E−06 | — | >0.05 |
| | acid binding | | | | | | |
| GO: 0090130 | tissue migration | 33/810 | 1.65E−04 | 38/941 | 6.29E−05 | — | >0.05 |
| GO: 0090132 | epithelium migration | 33/810 | 1.29E−04 | 38/941 | 4.73E−05 | — | >0.05 |
| GO: 0090150 | establishment of protein localization | 22/810 | 3.45E−03 | — | >0.05 | — | >0.05 |
| | to membrane | | | | | | |
| GO: 0090169 | regulation of spindle assembly | 7/810 | 4.96E−04 | 7/941 | 1.20E−03 | — | >0.05 |
| GO: 0090174 | organelle membrane fusion | 14/810 | 9.77E−04 | 14/941 | 3.89E−03 | — | >0.05 |
| GO: 0090224 | regulation of spindle organization | 8/810 | 9.46E−04 | 8/941 | 2.45E−03 | — | >0.05 |
| GO: 0090307 | mitotic spindle assembly | 10/810 | 1.19E−03 | 12/941 | 2.74E−04 | — | >0.05 |
| GO: 0090316 | positive regulation of intracellular | 15/810 | 3.39E−03 | 18/941 | 9.29E−04 | — | >0.05 |
| | protein transport | | | | | | |
| GO: 0090322 | regulation of superoxide metabolic | 9/810 | 1.18E− | 9/941 | 3.86E−05 | 11/1792 | 2.55E−04 |
| | process | | 05 | | | | |
| GO: 0090398 | cellular senescence | 16/810 | 1.80E−05 | 18/941 | 7.83E−06 | 22/1792 | 5.77E−04 |
| GO: 0090571 | RNA polymerase II transcription | 4/820 | 3.09E−03 | 4/959 | 5.42E−03 | — | >0.05 |
| | repressor complex | | | | | | |
| GO: 0090575 | RNA polymerase II transcription | 26/820 | 3.12E−05 | 26/959 | 3.83E−04 | — | >0.05 |
| | regulator complex | | | | | | |
| GO: 0090596 | sensory organ morphogenesis | — | >0.05 | — | >0.05 | 42/1792 | 1.53E−03 |
| GO: 0090734 | site of DNA damage | 11/820 | 6.89E−03 | 12/959 | 8.21E−03 | - | >0.05 |
| GO: 0095500 | acetylcholine receptor signaling | — | >0.05 | — | >0.05 | 10/1792 | 4.97E−04 |
| | pathway | | | | | | |
| GO: 0097060 | synaptic membrane | — | >0.05 | — | >0.05 | 61/1866 | 1.31E−04 |
| GO: 0097091 | synaptic vesicle clustering | — | >0.05 | 4/941 | 7.61E−03 | — | >0.05 |
| GO: 0097110 | scaffold protein binding | 9/812 | 2.59E−03 | — | >0.05 | — | >0.05 |
| GO: 0097152 | mesenchymal cell apoptotic | — | >0.05 | 4/941 | 5.95E−03 | 8/1792 | 2.78E−05 |
| | process | | | | | | |
| GO: 0097157 | pre-mRNA intronic binding | — | >0.05 | 4/949 | 3.64E−03 | — | >0.05 |
| GO: 0097178 | ruffle assembly | 8/810 | 5.10E−04 | 8/941 | 1.35E−03 | — | >0.05 |
| GO: 0097191 | extrinsic apoptotic signaling | 26/810 | 8.60E−06 | 26/941 | 1.08E−04 | — | >0.05 |
| | pathway | | | | | | |
| GO: 0097193 | intrinsic apoptotic signaling pathway | 37/810 | 1.78E−08 | 36/941 | 2.05E−06 | — | >0.05 |
| GO: 0097212 | lysosomal membrane organization | 6/810 | 2.57E−05 | 6/941 | 5.99E−05 | 6/1792 | 1.97E−03 |
| GO: 0097224 | sperm connecting piece | — | >0.05 | 3/959 | 1.52E−02 | — | >0.05 |
| GO: 0097225 | sperm midpiece | — | >0.05 | 7/959 | 1.04E−02 | — | >0.05 |
| GO: 0097237 | cellular response to toxic substance | 13/810 | 3.28E−03 | 14/941 | 4.51E−03 | — | >0.05 |
| GO: 0097305 | response to alcohol | 21/810 | 3.85E−03 | — | >0.05 | — | >0.05 |
| GO: 0097352 | autophagosome maturation | 9/810 | 9.25E−04 | 9/941 | 2.60E−03 | — | >0.05 |
| GO: 0097371 | MDM2/MDM4 family protein binding | 5/812 | 6.55E−05 | 5/949 | 1.38E−04 | — | >0.05 |
| GO: 0097479 | synaptic vesicle localization | — | >0.05 | 8/941 | 5.92E−03 | — | >0.05 |
| GO: 0097485 | neuron projection guidance | — | >0.05 | — | >0.05 | 38/1792 | 5.68E−04 |
| GO: 0097517 | contractile actin filament bundle | 8/820 | 8.96E−03 | — | >0.05 | — | >0.05 |
| GO: 0097529 | myeloid leukocyte migration | 24/810 | 1.65E−04 | 25/941 | 6.18E−04 | — | >0.05 |
| GO: 0097530 | granulocyte migration | 17/810 | 5.00E−04 | 17/941 | 2.53E−03 | — | >0.05 |
| GO: 0097531 | mast cell migration | 4/810 | 3.48E−03 | 4/941 | 5.95E−03 | 6/1792 | 1.97E−03 |
| GO: 0097576 | vacuole fusion | 5/810 | 1.01E−04 | 5/941 | 2.05E−04 | — | >0.05 |
| GO: 0097581 | lamellipodium organization | 13/810 | 1.73E−04 | 13/941 | 7.27E−04 | — | >0.05 |
| GO: 0097718 | disordered domain specific binding | 11/812 | 2.91E−07 | 11/949 | 1.36E−06 | 11/1815 | 5.51E−04 |
| GO: 0098553 | lumenal side of endoplasmic | 5/820 | 7.10E−03 | 5/959 | 1.34E−02 | — | >0.05 |
| | reticulum membrane | | | | | | |
| GO: 0098562 | cytoplasmic side of membrane | 27/820 | 5.12E−08 | 30/959 | 2.67E−08 | 40/1866 | 2.71E−06 |
| GO: 0098576 | lumenal side of membrane | — | >0.05 | 6/959 | 8.23E−03 | — | >0.05 |
| GO: 0098685 | Schaffer collateral - CA1 synapse | — | >0.05 | — | >0.05 | 19/1866 | 1.56E−03 |
| GO: 0098687 | chromosomal region | 41/820 | 9.97E−08 | 43/959 | 9.30E−07 | — | >0.05 |
| GO: 0098742 | cell-cell adhesion via plasma- | — | >0.05 | — | >0.05 | 47/1792 | 1.33E−04 |
| | membrane adhesion molecules | | | | | | |
| GO: 0098751 | bone cell development | 8/810 | 3.63E−04 | 8/941 | 9.76E−04 | — | >0.05 |
| GO: 0098754 | detoxification | 14/810 | 4.63E−03 | 16/941 | 2.93E−03 | — | >0.05 |
| GO: 0098760 | response to interleukin-7 | 6/810 | 2.57E−05 | 6/941 | 5.99E−05 | 6/1792 | 1.97E−03 |
| GO: 0098761 | cellular response to interleukin-7 | 6/810 | 2.57E−05 | 6/941 | 5.99E−05 | 6/1792 | 1.97E−03 |
| GO: 0098798 | mitochondrial protein-containing | 23/820 | 2.58E−03 | 25/959 | 4.42E−03 | — | >0.05 |
| | complex | | | | | | |
| GO: 0098800 | inner mitochondrial membrane | 18/820 | 3.57E−05 | 20/959 | 2.64E−05 | — | >0.05 |
| | protein complex | | | | | | |
| GO: 0098803 | respiratory chain complex | 9/820 | 6.97E−03 | 10/959 | 6.37E−03 | — | >0.05 |
| GO: 0098839 | postsynaptic density membrane | — | >0.05 | — | >0.05 | 19/1866 | 4.11E−03 |
| GO: 0098852 | lytic vacuole membrane | 44/820 | 2.30E−08 | 45/959 | 7.27E−07 | 60/1866 | 9.70E−04 |
| GO: 0098857 | membrane microdomain | 35/820 | 4.80E−07 | 38/959 | 9.40E−07 | 56/1866 | 1.48E−05 |
| GO: 0098858 | actin-based cell projection | 17/820 | 1.66E−02 | — | >0.05 | — | >0.05 |
| GO: 0098869 | cellular oxidant detoxification | 12/810 | 1.37E−03 | 13/941 | 1.61E−03 | — | >0.05 |
| GO: 0098926 | postsynaptic signal transduction | 6/810 | 6.80E−03 | 8/941 | 6.88E−04 | 13/1792 | 4.99E−05 |
| GO: 0098978 | glutamatergic synapse | 33/820 | 3.86E−04 | 38/959 | 1.91E−04 | 73/1866 | 2.46E−07 |
| GO: 0098984 | neuron to neuron synapse | 33/820 | 1.00E−04 | 37/959 | 9.00E−05 | 75/1866 | 2.08E−09 |
| GO: 0099010 | modification of postsynaptic | 4/810 | 7.01E−03 | 5/941 | 1.71E−03 | — | >0.05 |
| | structure | | | | | | |
| GO: 0099054 | presynapse assembly | — | >0.05 | 8/941 | 3.21E−03 | 12/1792 | 2.16E−03 |
| GO: 0099072 | regulation of postsynaptic | — | >0.05 | — | >0.05 | 19/1792 | 7.13E−04 |
| | membrane neurotransmitter | | | | | | |
| | receptor levels | | | | | | |
| GO: 0099172 | presynapse organization | — | >0.05 | 8/941 | 5.27E−03 | — | >0.05 |
| GO: 0099177 | regulation of trans-synaptic | — | >0.05 | — | >0.05 | 69/1792 | 1.07E−03 |
| | signaling | | | | | | |
| GO: 0099501 | exocytic vesicle membrane | — | >0.05 | 14/959 | 3.59E−03 | 26/1866 | 1.28E−04 |
| GO: 0099572 | postsynaptic specialization | 33/820 | 3.91E−05 | 37/959 | 3.24E−05 | 74/1866 | 5.26E−10 |
| GO: 0099590 | neurotransmitter receptor | — | >0.05 | — | >0.05 | 10/1792 | 3.69E−04 |
| | internalization | | | | | | |
| GO: 0099601 | regulation of neurotransmitter | — | >0.05 | — | >0.05 | 15/1792 | 8.75E−04 |
| | receptor activity | | | | | | |
| GO: 0099634 | postsynaptic specialization | — | >0.05 | — | >0.05 | 25/1866 | 6.03E−04 |
| | membrane | | | | | | |
| GO: 0101002 | ficolin-1-rich granule | 39/820 | 7.37E−17 | 41/959 | 4.23E−16 | 44/1866 | 1.56E−08 |
| GO: 0101003 | ficolin-1-rich granule membrane | 7/820 | 1.49E−02 | 8/959 | 1.06E−02 | — | >0.05 |
| GO: 0101031 | chaperone complex | 6/820 | 8.41E−03 | — | >0.05 | — | >0.05 |
| GO: 0104004 | cellular response to environmental | 26/810 | 4.23E−03 | 30/941 | 2.40E−03 | — | >0.05 |
| | stimulus | | | | | | |
| GO: 0106310 | protein serine kinase activity | — | >0.05 | 31/949 | 4.97E−03 | — | >0.05 |
| GO: 0110053 | regulation of actin filament | 24/810 | 7.89E−04 | 25/941 | 2.75E−03 | — | >0.05 |
| | organization | | | | | | |
| GO: 0120032 | regulation of plasma membrane | 19/810 | 9.23E−04 | 19/941 | 4.93E−03 | — | >0.05 |
| | bounded cell projection assembly | | | | | | |
| GO: 0120034 | positive regulation of plasma | 12/810 | 3.11E−03 | — | >0.05 | — | >0.05 |
| | membrane bounded cell projection | | | | | | |
| | assembly | | | | | | |
| GO: 0120111 | neuron projection cytoplasm | 9/820 | 1.37E−02 | 10/959 | 1.32E−02 | — | >0.05 |
| GO: 0140030 | modification-dependent protein | 18/812 | 8.84E−04 | 19/949 | 2.08E−03 | — | >0.05 |
| | binding | | | | | | |
| GO: 0140104 | molecular carrier activity | 10/812 | 5.89E−03 | — | >0.05 | — | >0.05 |
| GO: 0140142 | nucleocytoplasmic carrier activity | 6/812 | 1.87E−03 | 6/949 | 4.07E−03 | — | >0.05 |
| GO: 0140253 | cell-cell fusion | — | >0.05 | 9/941 | 4.14E−03 | — | >0.05 |
| GO: 0140297 | DNA-binding transcription factor | 49/812 | 5.48E−08 | 55/949 | 3.11E−08 | 74/1815 | 8.91E−05 |
| | binding | | | | | | |
| GO: 0140467 | integrated stress response | 6/810 | 8.70E−03 | — | >0.05 | — | >0.05 |
| | signaling | | | | | | |
| GO: 0140546 | defense response to symbiont | 31/810 | 1.10E−05 | 31/941 | 1.80E−04 | — | >0.05 |
| GO: 0140693 | molecular condensate scaffold | — | >0.05 | 5/949 | 2.44E−03 | — | >0.05 |
| | activity | | | | | | |
| GO: 0140694 | non-membrane-bounded organelle | 38/810 | 3.84E−06 | 40/941 | 2.16E−05 | — | >0.05 |
| | assembly | | | | | | |
| GO: 0140888 | interferon-mediated signaling | 12/810 | 1.49E−03 | 12/941 | 5.07E−03 | — | >0.05 |
| | pathway | | | | | | |
| GO: 0150063 | visual system development | — | >0.05 | — | >0.05 | 63/1792 | 4.07E−05 |
| GO: 1900044 | regulation of protein K63-linked | 4/810 | 4.48E−03 | 5/941 | 9.52E−04 | — | >0.05 |
| | ubiquitination | | | | | | |
| GO: 1900076 | regulation of cellular response to | 9/810 | 4.70E−03 | — | >0.05 | — | >0.05 |
| | insulin stimulus | | | | | | |
| GO: 1900078 | positive regulation of cellular | 7/810 | 1.66E−04 | 7/941 | 4.17E−04 | — | >0.05 |
| | response to insulin stimulus | | | | | | |
| GO: 1900087 | positive regulation of G1/S | 9/810 | 6.22E−04 | 9/941 | 1.78E−03 | — | >0.05 |
| | transition of mitotic cell cycle | | | | | | |
| GO: 1900151 | regulation of nuclear-transcribed | 5/810 | 5.02E−03 | — | >0.05 | — | >0.05 |
| | mRNA catabolic process, | | | | | | |
| | deadenylation-dependent decay | | | | | | |
| GO: 1900153 | positive regulation of nuclear- | 4/810 | 3.48E−03 | 4/941 | 5.95E−03 | — | >0.05 |
| | transcribed mRNA catabolic | | | | | | |
| | process, deadenylation-dependent | | | | | | |
| | decay | | | | | | |
| GO: 1900180 | regulation of protein localization to | 17/810 | 8.58E−05 | 19/941 | 5.35E−05 | 26/1792 | 4.96E−04 |
| | nucleus | | | | | | |
| GO: 1900182 | positive regulation of protein | 12/810 | 3.81E−04 | 14/941 | 1.13E−04 | 18/1792 | 1.42E−03 |
| | localization to nucleus | | | | | | |
| GO: 1900246 | positive regulation of RIG-I | 3/810 | 8.14E−03 | — | >0.05 | — | >0.05 |
| | signaling pathway | | | | | | |
| GO: 1900272 | negative regulation of long-term | — | >0.05 | 4/941 | 2.44E−03 | — | >0.05 |
| | synaptic potentiation | | | | | | |
| GO: 1900407 | regulation of cellular response to | 13/810 | 2.41E−04 | 15/941 | 8.25E−05 | — | >0.05 |
| | oxidative stress | | | | | | |
| GO: 1900542 | regulation of purine nucleotide | 14/810 | 3.79E−05 | 15/941 | 4.92E−05 | — | >0.05 |
| | metabolic process | | | | | | |
| GO: 1900543 | negative regulation of purine | 7/810 | 5.68E−05 | 8/941 | 1.63E−05 | 8/1792 | 1.43E−03 |
| | nucleotide metabolic process | | | | | | |
| GO: 1901214 | regulation of neuron death | 31/810 | 4.88E−05 | 33/941 | 1.47E−04 | 55/1792 | 3.63E−05 |
| GO: 1901215 | negative regulation of neuron death | 19/810 | 3.56E−03 | 21/941 | 3.94E−03 | 38/1792 | 2.73E−04 |
| GO: 1901216 | positive regulation of neuron death | 12/810 | 7.10E−04 | 12/941 | 2.54E−03 | 19/1792 | 1.26E−03 |
| GO: 1901222 | regulation of NIK/NF-kappaB | 14/810 | 3.10E−04 | 15/941 | 4.41E−04 | — | >0.05 |
| | signaling | | | | | | |
| GO: 1901223 | negative regulation of NIK/NF- | 6/810 | 8.70E−03 | — | >0.05 | — | >0.05 |
| | kappaB signaling | | | | | | |
| GO: 1901224 | positive regulation of NIK/NF- | 8/810 | 8.30E−03 | — | >0.05 | — | >0.05 |
| | kappaB signaling | | | | | | |
| GO: 1901342 | regulation of vasculature | 26/810 | 8.19E−03 | 31/941 | 2.76E−03 | — | >0.05 |
| | development | | | | | | |
| GO: 1901653 | cellular response to peptide | 39/810 | 6.47E−07 | 43/941 | 6.77E−07 | 64/1792 | 6.55E−06 |
| GO: 1901673 | regulation of mitotic spindle | 7/810 | 4.18E−05 | 7/941 | 1.08E−04 | — | >0.05 |
| | assembly | | | | | | |
| GO: 1901739 | regulation of myoblast fusion | — | >0.05 | 6/941 | 4.98E−04 | — | >0.05 |
| GO: 1901741 | positive regulation of myoblast | — | >0.05 | 5/941 | 9.52E−04 | — | >0.05 |
| | fusion | | | | | | |
| GO: 1901796 | regulation of signal transduction by | 14/810 | 2.09E−04 | 15/941 | 2.93E−04 | — | >0.05 |
| | p53 class mediator | | | | | | |
| GO: 1901798 | positive regulation of signal | 7/810 | 2.65E−04 | 7/941 | 6.56E−04 | — | >0.05 |
| | transduction by p53 class mediator | | | | | | |
| GO: 1901799 | negative regulation of proteasomal | 9/810 | 3.48E−04 | 8/941 | 4.14E−03 | — | >0.05 |
| | protein catabolic process | | | | | | |
| GO: 1901800 | positive regulation of proteasomal | 13/810 | 1.68E−03 | 13/941 | 6.02E−03 | — | >0.05 |
| | protein catabolic process | | | | | | |
| GO: 1901863 | positive regulation of muscle tissue | — | >0.05 | 5/941 | 6.59E−03 | — | >0.05 |
| | development | | | | | | |
| GO: 1901981 | phosphatidylinositol phosphate | 18/812 | 1.15E−03 | 19/949 | 2.69E−03 | — | >0.05 |
| | binding | | | | | | |
| GO: 1901987 | regulation of cell cycle phase | 39/810 | 5.83E−05 | 44/941 | 3.88E−05 | — | >0.05 |
| | transition | | | | | | |
| GO: 1901989 | positive regulation of cell cycle | 14/810 | 9.00E−04 | 16/941 | 4.55E−04 | — | >0.05 |
| | phase transition | | | | | | |
| GO: 1901990 | regulation of mitotic cell cycle | 35/810 | 8.46E−06 | 38/941 | 1.59E−05 | — | >0.05 |
| | phase transition | | | | | | |
| GO: 1901992 | positive regulation of mitotic cell | 13/810 | 4.05E−04 | 14/941 | 5.12E−04 | — | >0.05 |
| | cycle phase transition | | | | | | |
| GO: 1902041 | regulation of extrinsic apoptotic | 10/810 | 5.46E−05 | 10/941 | 1.89E−04 | — | >0.05 |
| | signaling pathway via death domain | | | | | | |
| | receptors | | | | | | |
| GO: 1902042 | negative regulation of extrinsic | 7/810 | 3.29E−04 | 7/941 | 8.10E−04 | — | >0.05 |
| | apoptotic signaling pathway via | | | | | | |
| | death domain receptors | | | | | | |
| GO: 1902074 | response to salt | — | >0.05 | — | >0.05 | 65/1792 | 5.63E−06 |
| GO: 1902075 | cellular response to salt | 18/810 | 3.92E−03 | 22/941 | 7.57E−04 | 42/1792 | 2.07E−06 |
| GO: 1902105 | regulation of leukocyte | 28/810 | 4.41E−04 | 30/941 | 1.03E−03 | — | >0.05 |
| | differentiation | | | | | | |
| GO: 1902107 | positive regulation of leukocyte | 16/810 | 7.58E−03 | — | >0.05 | — | >0.05 |
| | differentiation | | | | | | |
| GO: 1902115 | regulation of organelle assembly | 19/810 | 1.88E−03 | 20/941 | 4.42E−03 | — | >0.05 |
| GO: 1902175 | regulation of oxidative stress- | 9/810 | 2.74E−06 | 9/941 | 9.29E−06 | 9/1792 | 1.29E−03 |
| | induced intrinsic apoptotic signaling | | | | | | |
| | pathway | | | | | | |
| GO: 1902176 | negative regulation of oxidative | 6/810 | 1.20E−04 | 6/941 | 2.72E−04 | — | >0.05 |
| | stress-induced intrinsic apoptotic | | | | | | |
| | signaling pathway | | | | | | |
| GO: 1902369 | negative regulation of RNA | 10/810 | 3.82E−03 | 12/941 | 1.15E−03 | — | >0.05 |
| | catabolic process | | | | | | |
| GO: 1902373 | negative regulation of mRNA | 10/810 | 1.19E−03 | 12/941 | 2.74E−04 | — | >0.05 |
| | catabolic process | | | | | | |
| GO: 1902410 | mitotic cytokinetic process | 6/810 | 4.92E−04 | 6/941 | 1.08E−03 | — | >0.05 |
| GO: 1902495 | transmembrane transporter | — | >0.05 | — | >0.05 | 59/1866 | 2.53E−04 |
| | complex | | | | | | |
| GO: 1902600 | proton transmembrane transport | 18/810 | 2.74E−05 | 23/941 | 3.55E−07 | 26/1792 | 5.59E−04 |
| GO: 1902743 | regulation of lamellipodium | 11/810 | 1.94E−05 | 11/941 | 7.62E−05 | — | >0.05 |
| | organization | | | | | | |
| GO: 1902745 | positive regulation of lamellipodium | 8/810 | 1.71E−04 | 8/941 | 4.73E−04 | — | >0.05 |
| | organization | | | | | | |
| GO: 1902774 | late endosome to lysosome | 6/810 | 2.94E−04 | 6/941 | 6.56E−04 | — | >0.05 |
| | transport | | | | | | |
| GO: 1902806 | regulation of cell cycle G1/S phase | 25/810 | 7.64E−06 | 27/941 | 1.21E−05 | — | >0.05 |
| | transition | | | | | | |
| GO: 1902807 | negative regulation of cell cycle | 11/810 | 3.07E−03 | — | >0.05 | — | >0.05 |
| | G1/S phase transition | | | | | | |
| GO: 1902808 | positive regulation of cell cycle | 10/810 | 7.55E−04 | 11/941 | 6.34E−04 | — | >0.05 |
| | G1/S phase transition | | | | | | |
| GO: 1902850 | microtubule cytoskeleton | 16/810 | 2.06E−03 | 18/941 | 1.55E−03 | — | >0.05 |
| | organization involved in mitosis | | | | | | |
| GO: 1902882 | regulation of response to oxidative | 15/810 | 4.48E−05 | 17/941 | 1.80E−05 | 20/1792 | 2.09E−03 |
| | stress | | | | | | |
| GO: 1902893 | regulation of miRNA transcription | 10/810 | 7.55E−04 | 11/941 | 6.34E−04 | 16/1792 | 6.86E−04 |
| GO: 1902895 | positive regulation of miRNA | 9/810 | 3.48E−04 | 9/941 | 1.02E−03 | — | >0.05 |
| | transcription | | | | | | |
| GO: 1902903 | regulation of supramolecular fiber | 32/810 | 2.95E−04 | 33/941 | 1.81E−03 | — | >0.05 |
| | organization | | | | | | |
| GO: 1902904 | negative regulation of | 18/810 | 3.98E−04 | 18/941 | 2.18E−03 | — | >0.05 |
| | supramolecular fiber organization | | | | | | |
| GO: 1902914 | regulation of protein | 6/810 | 1.73E−03 | 7/941 | 6.56E−04 | — | >0.05 |
| | polyubiquitination | | | | | | |
| GO: 1902916 | positive regulation of protein | 4/810 | 5.65E−03 | 5/941 | 1.29E−03 | — | >0.05 |
| | polyubiquitination | | | | | | |
| GO: 1902936 | phosphatidylinositol bisphosphate | 12/812 | 3.00E−03 | 13/949 | 3.86E−03 | — | >0.05 |
| | binding | | | | | | |
| GO: 1903008 | organelle disassembly | 14/810 | 2.95E−03 | — | >0.05 | — | >0.05 |
| GO: 1903037 | regulation of leukocyte cell-cell | 34/810 | 6.66E−05 | 36/941 | 2.55E−04 | — | >0.05 |
| | adhesion | | | | | | |
| GO: 1903038 | negative regulation of leukocyte | 15/810 | 2.63E−03 | 16/941 | 4.40E−03 | — | >0.05 |
| | cell-cell adhesion | | | | | | |
| GO: 1903039 | positive regulation of leukocyte cell- | 28/810 | 2.91E−05 | 30/941 | 6.65E−05 | — | >0.05 |
| | cell adhesion | | | | | | |
| GO: 1903050 | regulation of proteolysis involved in | 31/810 | 2.50E−08 | 30/941 | 2.06E−06 | 39/1792 | 3.73E−04 |
| | protein catabolic process | | | | | | |
| GO: 1903051 | negative regulation of proteolysis | 11/810 | 1.34E−04 | 10/941 | 1.85E−03 | — | >0.05 |
| | involved in protein catabolic | | | | | | |
| | process | | | | | | |
| GO: 1903052 | positive regulation of proteolysis | 17/810 | 9.42E−05 | 17/941 | 5.45E−04 | — | >0.05 |
| | involved in protein catabolic | | | | | | |
| | process | | | | | | |
| GO: 1903076 | regulation of protein localization to | 14/810 | 1.70E−04 | 17/941 | 1.80E−05 | 21/1792 | 8.54E−04 |
| | plasma membrane | | | | | | |
| GO: 1903077 | negative regulation of protein | 6/810 | 4.92E−04 | 8/941 | 1.63E−05 | 9/1792 | 2.64E−04 |
| | localization to plasma membrane | | | | | | |
| GO: 1903078 | positive regulation of protein | 8/810 | 4.66E−03 | 9/941 | 3.30E−03 | — | >0.05 |
| | localization to plasma membrane | | | | | | |
| GO: 1903131 | mononuclear cell differentiation | 43/810 | 6.16E−06 | 45/941 | 4.98E−05 | — | >0.05 |
| GO: 1903146 | regulation of autophagy of | 7/810 | 1.02E−03 | 7/941 | 2.43E−03 | — | >0.05 |
| | mitochondrion | | | | | | |
| GO: 1903201 | regulation of oxidative stress- | 12/810 | 1.69E−04 | 13/941 | 1.76E−04 | — | >0.05 |
| | induced cell death | | | | | | |
| GO: 1903202 | negative regulation of oxidative | 9/810 | 6.22E−04 | 10/941 | 4.26E−04 | — | >0.05 |
| | stress-induced cell death | | | | | | |
| GO: 1903203 | regulation of oxidative stress- | 7/810 | 3.29E−04 | 7/941 | 8.10E−04 | — | >0.05 |
| | induced neuron death | | | | | | |
| GO: 1903204 | negative regulation of oxidative | 5/810 | 1.86E−03 | 5/941 | 3.58E−03 | — | >0.05 |
| | stress-induced neuron death | | | | | | |
| GO: 1903265 | positive regulation of tumor | 4/810 | 1.95E−03 | 4/941 | 3.38E−03 | — | >0.05 |
| | necrosis factor-mediated signaling | | | | | | |
| | pathway | | | | | | |
| GO: 1903293 | phosphatase complex | 7/820 | 7.79E−03 | 8/959 | 5.08E−03 | — | >0.05 |
| GO: 1903306 | negative regulation of regulated | 6/810 | 7.81E−04 | 6/941 | 1.70E−03 | — | >0.05 |
| | secretory pathway | | | | | | |
| GO: 1903311 | regulation of mRNA metabolic | 38/810 | 5.22E−08 | 40/941 | 3.10E−07 | 49/1792 | 1.62E−03 |
| | process | | | | | | |
| GO: 1903312 | negative regulation of mRNA | 16/810 | 3.75E−06 | 18/941 | 1.34E−06 | 21/1792 | 2.73E−04 |
| | metabolic process | | | | | | |
| GO: 1903320 | regulation of protein modification by | 39/810 | 7.38E−12 | 40/941 | 1.70E−10 | 43/1792 | 2.04E−04 |
| | small protein conjugation or | | | | | | |
| | removal | | | | | | |
| GO: 1903321 | negative regulation of protein | 19/810 | 3.27E−08 | 19/941 | 3.38E−07 | 21/1792 | 3.18E−04 |
| | modification by small protein | | | | | | |
| | conjugation or removal | | | | | | |
| GO: 1903322 | positive regulation of protein | 16/810 | 3.59E−04 | 17/941 | 6.47E−04 | — | >0.05 |
| | modification by small protein | | | | | | |
| | conjugation or removal | | | | | | |
| GO: 1903531 | negative regulation of secretion by | 15/810 | 3.83E−03 | 16/941 | 6.41E−03 | 29/1792 | 5.11E−04 |
| | cell | | | | | | |
| GO: 1903532 | positive regulation of secretion by | 26/810 | 4.80E−04 | 29/941 | 4.41E−04 | 46/1792 | 6.20E−04 |
| | cell | | | | | | |
| GO: 1903541 | regulation of exosomal secretion | 4/810 | 5.65E−03 | — | >0.05 | — | >0.05 |
| GO: 1903543 | positive regulation of exosomal | 4/810 | 3.48E−03 | 4/941 | 5.95E−03 | — | >0.05 |
| | secretion | | | | | | |
| GO: 1903555 | regulation of tumor necrosis factor | 19/810 | 7.16E−04 | — | >0.05 | — | >0.05 |
| | superfamily cytokine production | | | | | | |
| GO: 1903557 | positive regulation of tumor | 12/810 | 2.88E−03 | — | >0.05 | — | >0.05 |
| | necrosis factor superfamily cytokine | | | | | | |
| | production | | | | | | |
| GO: 1903578 | regulation of ATP metabolic | 12/810 | 6.73E−05 | 12/941 | 2.74E−04 | — | >0.05 |
| | process | | | | | | |
| GO: 1903579 | negative regulation of ATP | 6/810 | 2.22E−04 | 6/941 | 4.98E−04 | — | >0.05 |
| | metabolic process | | | | | | |
| GO: 1903649 | regulation of cytoplasmic transport | 5/810 | 8.12E−03 | 6/941 | 3.07E−03 | — | >0.05 |
| GO: 1903706 | regulation of hemopoiesis | 40/810 | 2.82E−06 | 43/941 | 8.38E−06 | — | >0.05 |
| GO: 1903708 | positive regulation of hemopoiesis | 16/810 | 7.58E−03 | — | >0.05 | — | >0.05 |
| GO: 1903715 | regulation of aerobic respiration | 6/810 | 2.89E−03 | 6/941 | 6.02E−03 | — | >0.05 |
| GO: 1903729 | regulation of plasma membrane | 4/810 | 7.01E−03 | — | >0.05 | — | >0.05 |
| | organization | | | | | | |
| GO: 1903828 | negative regulation of protein | 24/810 | 1.96E−05 | 26/941 | 2.87E−05 | 40/1792 | 2.81E−05 |
| | localization | | | | | | |
| GO: 1903829 | positive regulation of protein | 43/810 | 4.46E−06 | 47/941 | 8.01E−06 | — | >0.05 |
| | localization | | | | | | |
| GO: 1903846 | positive regulation of cellular | — | >0.05 | 7/941 | 1.45E−03 | — | >0.05 |
| | response to transforming growth | | | | | | |
| | factor beta stimulus | | | | | | |
| GO: 1903900 | regulation of viral life cycle | 21/810 | 1.25E−06 | 22/941 | 3.70E−06 | — | >0.05 |
| GO: 1903902 | positive regulation of viral life cycle | 5/810 | 3.49E−03 | 5/941 | 6.59E−03 | — | >0.05 |
| GO: 1904018 | positive regulation of vasculature | 16/810 | 7.97E−03 | 19/941 | 3.10E−03 | — | >0.05 |
| | development | | | | | | |
| GO: 1904029 | regulation of cyclin-dependent | 13/810 | 1.55E−03 | 13/941 | 5.59E−03 | — | >0.05 |
| | protein kinase activity | | | | | | |
| GO: 1904030 | negative regulation of cyclin- | 6/810 | 2.89E−03 | 6/941 | 6.02E−03 | — | >0.05 |
| | dependent protein kinase activity | | | | | | |
| GO: 1904115 | axon cytoplasm | — | >0.05 | 8/959 | 1.28E−02 | — | >0.05 |
| GO: 1904356 | regulation of telomere maintenance | 9/810 | 1.34E−03 | 9/941 | 3.70E−03 | — | >0.05 |
| | via telomere lengthening | | | | | | |
| GO: 1904358 | positive regulation of telomere | 7/810 | 1.02E−03 | 7/941 | 2.43E−03 | — | >0.05 |
| | maintenance via telomere | | | | | | |
| | lengthening | | | | | | |
| GO: 1904375 | regulation of protein localization to | 16/810 | 1.64E−04 | 19/941 | 2.80E−05 | 25/1792 | 5.70E−04 |
| | cell periphery | | | | | | |
| GO: 1904376 | negative regulation of protein | 6/810 | 7.81E−04 | 8/941 | 3.16E−05 | 9/1792 | 5.28E−04 |
| | localization to cell periphery | | | | | | |
| GO: 1904377 | positive regulation of protein | 9/810 | 2.90E−03 | 10/941 | 2.32E−03 | — | >0.05 |
| | localization to cell periphery | | | | | | |
| GO: 1904659 | glucose transmembrane transport | — | >0.05 | — | >0.05 | 22/1792 | 1.40E−03 |
| GO: 1904666 | regulation of ubiquitin protein ligase | 7/810 | 9.99E−05 | 7/941 | 2.53E−04 | — | >0.05 |
| | activity | | | | | | |
| GO: 1904667 | negative regulation of ubiquitin | 5/810 | 1.01E−04 | 5/941 | 2.05E−04 | — | >0.05 |
| | protein ligase activity | | | | | | |
| GO: 1904724 | tertiary granule lumen | 9/820 | 4.92E−04 | 8/959 | 5.69E−03 | — | >0.05 |
| GO: 1904813 | ficolin-1-rich granule lumen | 32/820 | 9.07E−17 | 33/959 | 1.07E−15 | 35/1866 | 4.22E−09 |
| GO: 1904896 | ESCRT complex disassembly | 5/810 | 3.46E−05 | 5/941 | 7.11E−05 | 5/1792 | 1.46E−03 |
| GO: 1904903 | ESCRT III complex disassembly | 5/810 | 3.46E−05 | 5/941 | 7.11E−05 | 5/1792 | 1.46E−03 |
| GO: 1904930 | amphisome membrane | 5/820 | 8.65E−05 | 5/959 | 1.81E−04 | 5/1866 | 3.81E−03 |
| GO: 1904949 | ATPase complex | 15/820 | 1.20E−03 | 19/959 | 9.31E−05 | — | >0.05 |
| GO: 1904951 | positive regulation of establishment | 30/810 | 1.10E−04 | 33/941 | 1.39E−04 | — | >0.05 |
| | of protein localization | | | | | | |
| GO: 1905144 | response to acetylcholine | — | >0.05 | — | >0.05 | 11/1792 | 4.45E−04 |
| GO: 1905145 | cellular response to acetylcholine | — | >0.05 | — | >0.05 | 10/1792 | 8.66E−04 |
| GO: 1905360 | GTPase complex | 6/820 | 1.75E−03 | 6/959 | 3.84E−03 | — | >0.05 |
| GO: 1905368 | peptidase complex | 17/820 | 2.34E−05 | 17/959 | 1.61E−04 | — | >0.05 |
| GO: 1905369 | endopeptidase complex | 15/820 | 5.81E−06 | 15/959 | 3.67E−05 | 17/1866 | 5.56E−03 |
| GO: 1905475 | regulation of protein localization to | 18/810 | 7.49E−04 | 21/941 | 2.53E−04 | 32/1792 | 3.67E−04 |
| | membrane | | | | | | |
| GO: 1905476 | negative regulation of protein | 6/810 | 2.89E−03 | 8/941 | 2.04E−04 | 11/1792 | 1.89E−04 |
| | localization to membrane | | | | | | |
| GO: 1905477 | positive regulation of protein | 11/810 | 5.32E−03 | 12/941 | 5.94E−03 | — | >0.05 |
| | localization to membrane | | | | | | |
| GO: 1905666 | regulation of protein localization to | 4/810 | 1.95E−03 | 4/941 | 3.38E−03 | — | >0.05 |
| | endosome | | | | | | |
| GO: 1990089 | response to nerve growth factor | 9/810 | 2.16E−04 | 9/941 | 6.48E−04 | — | >0.05 |
| GO: 1990090 | cellular response to nerve growth | 9/810 | 1.54E−04 | 9/941 | 4.67E−04 | — | >0.05 |
| | factor stimulus | | | | | | |
| GO: 1990124 | messenger ribonucleoprotein complex | 3/820 | 1.28E−02 | — | >0.05 | — | >0.05 |
| GO: 1990204 | oxidoreductase complex | 15/820 | 1.67E−04 | 16/959 | 2.83E−04 | — | >0.05 |
| GO: 1990226 | histone methyltransferase binding | 4/812 | 3.66E−03 | — | >0.05 | — | >0.05 |
| GO: 1990266 | neutrophil migration | 14/810 | 1.45E−03 | 14/941 | 5.58E−03 | — | >0.05 |
| GO: 1990351 | transporter complex | — | >0.05 | — | >0.05 | 61/1866 | 4.79E−04 |
| GO: 1990381 | ubiquitin-specific protease binding | 7/812 | 2.35E−05 | 7/949 | 6.39E−05 | — | >0.05 |
| GO: 1990748 | cellular detoxification | 13/810 | 1.68E−03 | 14/941 | 2.25E−03 | — | >0.05 |
| GO: 1990778 | protein localization to cell periphery | — | >0.05 | 29/941 | 5.37E−03 | — | >0.05 |
| GO: 2000045 | regulation of G1/S transition of | 24/810 | 2.41E−06 | 25/941 | 9.87E−06 | — | >0.05 |
| | mitotic cell cycle | | | | | | |
| GO: 2000058 | regulation of ubiquitin-dependent | 25/810 | 1.36E−07 | 24/941 | 7.01E−06 | 31/1792 | 5.24E−04 |
| | protein catabolic process | | | | | | |
| GO: 2000059 | negative regulation of ubiquitin- | 11/810 | 1.09E−05 | 10/941 | 2.24E−04 | 14/1792 | 2.72E−04 |
| | dependent protein catabolic | | | | | | |
| | process | | | | | | |
| GO: 2000060 | positive regulation of ubiquitin- | 14/810 | 3.10E−04 | 14/941 | 1.35E−03 | — | >0.05 |
| | dependent protein catabolic | | | | | | |
| | process | | | | | | |
| GO: 2000106 | regulation of leukocyte apoptotic | 13/810 | 1.55E−04 | 13/941 | 6.53E−04 | — | >0.05 |
| | process | | | | | | |
| GO: 2000116 | regulation of cysteine-type | 25/810 | 4.16E−05 | 26/941 | 1.77E−04 | — | >0.05 |
| | endopeptidase activity | | | | | | |
| GO: 2000117 | negative regulation of cysteine-type | 12/810 | 6.43E−04 | 13/941 | 7.27E−04 | — | >0.05 |
| | endopeptidase activity | | | | | | |
| GO: 2000134 | negative regulation of G1/S | 10/810 | 4.52E−03 | — | >0.05 | — | >0.05 |
| | transition of mitotic cell cycle | | | | | | |
| GO: 2000278 | regulation of DNA biosynthetic | 19/810 | 1.42E−06 | 19/941 | 1.24E−05 | — | >0.05 |
| | process | | | | | | |
| GO: 2000311 | regulation of AMPA receptor activity | — | >0.05 | — | >0.05 | 9/1792 | 3.78E−04 |
| GO: 2000377 | regulation of reactive oxygen | 19/810 | 3.13E−05 | 23/941 | 2.11E−06 | 31/1792 | 3.84E−05 |
| | species metabolic process | | | | | | |
| GO: 2000379 | positive regulation of reactive | 14/810 | 1.53E−06 | 16/941 | 2.95E−07 | 19/1792 | 2.24E−05 |
| | oxygen species metabolic process | | | | | | |
| GO: 2000434 | regulation of protein neddylation | 4/810 | 8.58E−03 | — | >0.05 | — | >0.05 |
| GO: 2000510 | positive regulation of dendritic cell | 3/810 | 8.14E−03 | — | >0.05 | — | >0.05 |
| | chemotaxis | | | | | | |
| GO: 2000573 | positive regulation of DNA | 12/810 | 8.86E−05 | 12/941 | 3.56E−04 | — | >0.05 |
| | biosynthetic process | | | | | | |
| GO: 2000628 | regulation of miRNA metabolic | 12/810 | 2.15E−04 | 13/941 | 2.28E−04 | 19/1792 | 2.35E−04 |
| | process | | | | | | |
| GO: 2000630 | positive regulation of miRNA | 11/810 | 5.45E−05 | 11/941 | 2.06E−04 | 15/1792 | 5.04E−04 |
| | metabolic process | | | | | | |
| GO: 2000641 | regulation of early endosome to late | 4/810 | 8.58E−03 | — | >0.05 | — | >0.05 |
| | endosome transport | | | | | | |
| GO: 2000677 | regulation of transcription regulatory | 8/810 | 1.86E−03 | 8/941 | 4.68E−03 | — | >0.05 |
| | region DNA binding | | | | | | |
| GO: 2000679 | positive regulation of transcription | 6/810 | 6.24E−04 | 6/941 | 1.36E−03 | — | >0.05 |
| | regulatory region DNA binding | | | | | | |
| GO: 2000696 | regulation of epithelial cell | — | >0.05 | — | >0.05 | 6/1792 | 2.90E−03 |
| | differentiation involved in kidney | | | | | | |
| | development | | | | | | |
| GO: 2000737 | negative regulation of stem cell | 5/810 | 5.94E−03 | 6/941 | 2.09E−03 | — | >0.05 |
| | differentiation | | | | | | |
| GO: 2000772 | regulation of cellular senescence | — | >0.05 | 8/941 | 5.92E−03 | — | >0.05 |
| GO: 2001053 | regulation of mesenchymal cell | — | >0.05 | 4/941 | 3.38E−03 | 8/1792 | 6.66E−06 |
| | apoptotic process | | | | | | |
| GO: 2001054 | negative regulation of | — | >0.05 | — | >0.05 | 7/1792 | 7.68E−06 |
| | mesenchymal cell apoptotic | | | | | | |
| | process | | | | | | |
| GO: 2001056 | positive regulation of cysteine-type | 15/810 | 1.53E−03 | 15/941 | 6.25E−03 | — | >0.05 |
| | endopeptidase activity | | | | | | |
| GO: 2001233 | regulation of apoptotic signaling | 50/810 | 4.86E−12 | 50/941 | 9.59E−10 | 61/1792 | 7.51E−05 |
| | pathway | | | | | | |
| GO: 2001234 | negative regulation of apoptotic | 34/810 | 8.17E−10 | 34/941 | 3.50E−08 | 41/1792 | 1.44E−04 |
| | signaling pathway | | | | | | |
| GO: 2001235 | positive regulation of apoptotic | 17/810 | 1.48E−04 | 17/941 | 8.29E−04 | — | >0.05 |
| | signaling pathway | | | | | | |
| GO: 2001236 | regulation of extrinsic apoptotic | 20/810 | 1.80E−05 | 20/941 | 1.44E−04 | — | >0.05 |
| | signaling pathway | | | | | | |
| GO: 2001237 | negative regulation of extrinsic | 14/810 | 1.10E−04 | 14/941 | 5.12E−04 | — | >0.05 |
| | apoptotic signaling pathway | | | | | | |
| GO: 2001238 | positive regulation of extrinsic | 7/810 | 6.76E−03 | — | >0.05 | — | >0.05 |
| | apoptotic signaling pathway | | | | | | |
| GO: 2001242 | regulation of intrinsic apoptotic | 27/810 | 1.05E−08 | 27/941 | 2.24E−07 | — | >0.05 |
| | signaling pathway | | | | | | |
| GO: 2001243 | negative regulation of intrinsic | 18/810 | 2.89E−07 | 18/941 | 2.50E−06 | — | >0.05 |
| | apoptotic signaling pathway | | | | | | |
| GO: 2001244 | positive regulation of intrinsic | 8/810 | 6.28E−03 | — | >0.05 | — | >0.05 |
| | apoptotic signaling pathway | | | | | | |

TABLE 6
KEGG pathways enriched in the expanded model pathway analyses for 40, 90 and 220 transcript models

| | | 40 transcripts | | 90 transcripts | | 220 transcripts | |
|---|---|---|---|---|---|---|---|
| | | model | | model | | model | |
| | | Transcript | | Transcript | | Transcript | |
| KEGG | KEGG term | number | | number | | number | |
| term ID | description | ratio | p-value | ratio | p-value | ratio | p-value |
| hsa00010 | Glycolysis/Gluconeogenesis | 9/522 | 2.20E−02 | — | >0.05 | — | >0.05 |
| hsa00190 | Oxidative phosphorylation | 18/522 | 5.45E−04 | 21/585 | 1.01E−04 | — | >0.05 |
| hsa01200 | Carbon metabolism | 15/522 | 5.62E−03 | — | >0.05 | — | >0.05 |
| hsa01230 | Biosynthesis of amino acids | 10/522 | 1.72E−02 | — | >0.05 | — | >0.05 |
| hsa03010 | Ribosome | 50/522 | 4.67E−23 | 50/585 | 7.79E−21 | 52/925 | 8.47E−14 |
| hsa03040 | Spliceosome | 20/522 | 2.33E−03 | 20/585 | 8.31E−03 | — | >0.05 |
| hsa03050 | Proteasome | 7/522 | 2.40E−02 | — | >0.05 | — | >0.05 |
| hsa04014 | Ras signaling pathway | 23/522 | 2.46E−02 | — | >0.05 | 40/925 | 4.54E−03 |
| hsa04015 | Rap1 signaling pathway | 23/522 | 6.69E−03 | — | >0.05 | — | >0.05 |
| hsa04022 | cGMP-PKG signaling pathway | — | >0.05 | — | >0.05 | 30/925 | 5.66E−03 |
| hsa04024 | cAMP signaling pathway | — | >0.05 | — | >0.05 | 41/925 | 1.02E−03 |
| hsa04062 | Chemokine signaling pathway | 28/522 | 2.28E−05 | 29/585 | 6.84E−05 | 37/925 | 5.44E−04 |
| hsa04066 | HIF-1 signaling pathway | 18/522 | 1.44E−04 | 19/585 | 2.02E−04 | 23/925 | 1.86E−03 |
| hsa04071 | Sphingolipid signaling pathway | 14/522 | 1.99E−02 | 18/585 | 2.01E−03 | 25/925 | 1.64E−03 |
| hsa04137 | Mitophagy - animal | 14/522 | 1.33E−04 | 15/585 | 1.22E−04 | — | >0.05 |
| hsa04140 | Autophagy - animal | 16/522 | 1.59E−02 | — | >0.05 | — | >0.05 |
| hsa04141 | Protein processing in | 19/522 | 1.19E−02 | 23/585 | 2.23E−03 | — | >0.05 |
| | endoplasmic reticulum | | | | | | |
| hsa04144 | Endocytosis | 40/522 | 3.95E−08 | 41/585 | 3.07E−07 | 52/925 | 6.29E−06 |
| hsa04145 | Phagosome | 21/522 | 4.94E−04 | 23/585 | 3.62E−04 | — | >0.05 |
| hsa04151 | PI3K-Akt signaling pathway | 34/522 | 8.28E−03 | 37/585 | 9.43E−03 | — | >0.05 |
| hsa04210 | Apoptosis | 17/522 | 5.14E−03 | 19/585 | 3.19E−03 | — | >0.05 |
| hsa04217 | Necroptosis | 33/522 | 6.39E−10 | 34/585 | 3.04E−09 | 37/925 | 8.02E−06 |
| hsa04218 | Cellular senescence | 26/522 | 4.28E−06 | 29/585 | 1.20E−06 | 32/925 | 4.51E−04 |
| hsa04370 | VEGF signaling pathway | — | >0.05 | 10/585 | 7.79E−03 | — | >0.05 |
| hsa04380 | Osteoclast differentiation | 23/522 | 3.64E−06 | 25/585 | 2.22E−06 | 30/925 | 4.78E−05 |
| hsa04390 | Hippo signaling pathway | — | >0.05 | — | >0.05 | 29/925 | 3.91E−03 |
| hsa04510 | Focal adhesion | 22/522 | 8.42E−03 | 25/585 | 4.22E−03 | 35/925 | 5.37E−03 |
| hsa04530 | Tight junction | 19/522 | 9.40E−03 | — | >0.05 | — | >0.05 |
| hsa04550 | Signaling pathways regulating | — | >0.05 | — | >0.05 | 27/925 | 4.23E−03 |
| | pluripotency of stem cells | | | | | | |
| hsa04611 | Platelet activation | 14/522 | 2.41E−02 | — | >0.05 | — | >0.05 |
| hsa04612 | Antigen processing and | 13/522 | 1.08E−03 | 13/585 | 3.00E−03 | — | >0.05 |
| | presentation | | | | | | |
| hsa04613 | Neutrophil extracellular trap | 34/522 | 8.65E−09 | 37/585 | 3.75E−09 | 39/925 | 4.67E−05 |
| | formation | | | | | | |
| hsa04621 | NOD-like receptor signaling | 21/522 | 6.65E−03 | — | >0.05 | — | >0.05 |
| | pathway | | | | | | |
| hsa04650 | Natural killer cell mediated | 18/522 | 1.41E−03 | 18/585 | 4.83E−03 | — | >0.05 |
| | cytotoxicity | | | | | | |
| hsa04659 | Th17 cell differentiation | 15/522 | 3.08E−03 | 16/585 | 3.65E−03 | — | >0.05 |
| hsa04662 | B cell receptor signaling | 16/522 | 4.29E−05 | 17/585 | 4.68E−05 | 19/925 | 1.44E−03 |
| | pathway | | | | | | |
| hsa04664 | Fc epsilon RI signaling | 12/522 | 8.67E−04 | 15/585 | 5.09E−05 | 19/925 | 8.95E−05 |
| | pathway | | | | | | |
| hsa04666 | Fc gamma R-mediated | 20/522 | 1.66E−06 | 21/585 | 2.49E−06 | 25/925 | 3.62E−05 |
| | phagocytosis | | | | | | |
| hsa04670 | Leukocyte transendothelial | 20/522 | 2.22E−05 | 21/585 | 3.57E−05 | 24/925 | 1.36E−03 |
| | migration | | | | | | |
| hsa04722 | Neurotrophin signaling | 16/522 | 3.17E−03 | 18/585 | 1.66E−03 | 23/925 | 5.92E−03 |
| | pathway | | | | | | |
| hsa04725 | Cholinergic synapse | — | >0.05 | — | >0.05 | 22/925 | 6.42E−03 |
| hsa04728 | Dopaminergic synapse | — | >0.05 | — | >0.05 | 25/925 | 5.01E−03 |
| hsa04810 | Regulation of actin | 24/522 | 9.19E−03 | — | >0.05 | — | >0.05 |
| | cytoskeleton | | | | | | |
| hsa04917 | Prolactin signaling pathway | 10/522 | 1.19E−02 | 12/585 | 3.37E−03 | — | >0.05 |
| hsa04928 | Parathyroid hormone | — | >0.05 | 15/585 | 7.37E−03 | 21/925 | 6.23E−03 |
| | synthesis, secretion and action | | | | | | |
| hsa04931 | Insulin resistance | 13/522 | 1.68E−02 | — | >0.05 | — | >0.05 |
| hsa04933 | AGE-RAGE signaling pathway | 12/522 | 2.31E−02 | 14/585 | 1.04E−02 | — | >0.05 |
| | in diabetic complications | | | | | | |
| hsa04935 | Growth hormone synthesis, | — | >0.05 | 16/585 | 1.01E−02 | — | >0.05 |
| | secretion and action | | | | | | |
| hsa04966 | Collecting duct acid secretion | — | >0.05 | 6/585 | 1.00E−02 | — | >0.05 |
| hsa05010 | Alzheimer disease | 38/522 | 1.98E−03 | 40/585 | 4.63E−03 | — | >0.05 |
| hsa05012 | Parkinson disease | 39/522 | 1.46E−07 | 42/585 | 1.38E−07 | 46/925 | 5.41E−04 |
| hsa05014 | Amyotrophic lateral sclerosis | 44/522 | 7.93E−06 | 45/585 | 6.18E−05 | — | >0.05 |
| hsa05016 | Huntington disease | 32/522 | 1.59E−03 | 33/585 | 5.05E−03 | — | >0.05 |
| hsa05020 | Prion disease | 35/522 | 1.50E−05 | 36/585 | 7.03E−05 | — | >0.05 |
| hsa05022 | Pathways of | 54/522 | 6.46E−06 | 58/585 | 1.07E−05 | 75/925 | 5.04E−04 |
| | neurodegeneration - multiple | | | | | | |
| | diseases | | | | | | |
| hsa05032 | Morphine addiction | — | >0.05 | — | >0.05 | 21/925 | 8.75E−04 |
| hsa05034 | Alcoholism | 31/522 | 2.73E−07 | 32/585 | 1.08E−06 | 35/925 | 7.87E−04 |
| hsa05100 | Bacterial invasion of epithelial | 16/522 | 1.89E−05 | 16/585 | 7.56E−05 | 17/925 | 4.29E−03 |
| | cells | | | | | | |
| hsa05130 | Pathogenic Escherichia coli | 27/522 | 9.58E−05 | 29/585 | 1.10E−04 | 36/925 | 1.74E−03 |
| | infection | | | | | | |
| hsa05131 | Shigellosis | 40/522 | 1.97E−08 | 43/585 | 1.83E−08 | 49/925 | 3.11E−05 |
| hsa05132 | Salmonella infection | 35/522 | 5.22E−06 | 39/585 | 1.61E−06 | 44/925 | 1.19E−03 |
| hsa05135 | Yersinia infection | 21/522 | 1.13E−04 | 24/585 | 2.47E−05 | 28/925 | 9.43E−04 |
| hsa05140 | Leishmaniasis | 14/522 | 2.43E−04 | 15/585 | 2.31E−04 | 18/925 | 1.46E−03 |
| hsa05145 | Toxoplasmosis | 13/522 | 2.37E−02 | 18/585 | 8.11E−04 | 23/925 | 2.69E−03 |
| hsa05152 | Tuberculosis | 21/522 | 4.27E−03 | 23/585 | 3.73E−03 | — | >0.05 |
| hsa05160 | Hepatitis C | 17/522 | 1.90E−02 | — | >0.05 | — | >0.05 |
| hsa05161 | Hepatitis B | 19/522 | 6.01E−03 | 21/585 | 4.57E−03 | — | >0.05 |
| hsa05162 | Measles | 18/522 | 2.54E−03 | 19/585 | 3.76E−03 | — | >0.05 |
| hsa05166 | Human T-cell leukemia virus 1 | 28/522 | 3.27E−04 | 29/585 | 9.23E−04 | — | >0.05 |
| | infection | | | | | | |
| hsa05167 | Kaposi sarcoma-associated | 21/522 | 1.06E−02 | 23/585 | 9.86E−03 | — | >0.05 |
| | herpesvirus infection | | | | | | |
| hsa05169 | Epstein-Barr virus infection | 28/522 | 5.33E−05 | 30/585 | 6.47E−05 | — | >0.05 |
| hsa05170 | Human immunodeficiency virus | 24/522 | 3.51E−03 | 26/585 | 3.78E−03 | — | >0.05 |
| | 1 infection | | | | | | |
| hsa05171 | Coronavirus disease - | 61/522 | 4.64E−23 | 62/585 | 3.51E−21 | 68/925 | 1.08E−14 |
| | COVID-19 | | | | | | |
| hsa05202 | Transcriptional misregulation in | 23/522 | 2.18E−03 | 26/585 | 9.82E−04 | — | >0.05 |
| | cancer | | | | | | |
| hsa05203 | Viral carcinogenesis | 37/522 | 1.83E−09 | 37/585 | 3.96E−08 | 38/925 | 6.16E−04 |
| hsa05205 | Proteoglycans in cancer | 24/522 | 2.40E−03 | 28/585 | 5.53E−04 | 40/925 | 2.82E−04 |
| hsa05207 | Chemical carcinogenesis - | 21/522 | 2.63E−02 | — | >0.05 | — | >0.05 |
| | receptor activation | | | | | | |
| hsa05208 | Chemical carcinogenesis - | 32/522 | 2.56E−06 | 36/585 | 4.97E−07 | 39/925 | 9.21E−04 |
| | reactive oxygen species | | | | | | |
| hsa05211 | Renal cell carcinoma | 12/522 | 1.14E−03 | 13/585 | 9.45E−04 | 17/925 | 1.23E−03 |
| hsa05212 | Pancreatic cancer | 11/522 | 7.76E−03 | 12/585 | 6.68E−03 | — | >0.05 |
| hsa05215 | Prostate cancer | 13/522 | 7.66E−03 | 14/585 | 7.97E−03 | — | >0.05 |
| hsa05220 | Chronic myeloid leukemia | 11/522 | 7.76E−03 | 12/585 | 6.68E−03 | — | >0.05 |
| hsa05221 | Acute myeloid leukemia | 10/522 | 8.81E−03 | 11/585 | 6.87E−03 | — | >0.05 |
| hsa05223 | Non-small cell lung cancer | 14/522 | 1.33E−04 | 14/585 | 4.35E−04 | 17/925 | 2.02E−03 |
| hsa05235 | PD-L1 expression and PD-1 | 13/522 | 3.66E−03 | 15/585 | 1.32E−03 | — | >0.05 |
| | checkpoint pathway in cancer | | | | | | |
| hsa05322 | Systemic lupus erythematosus | 26/522 | 9.33E−08 | 26/585 | 8.67E−07 | 27/925 | 8.89E−04 |
| hsa05415 | Diabetic cardiomyopathy | 32/522 | 2.60E−07 | 36/585 | 3.66E−08 | 40/925 | 5.02E−05 |
| hsa05416 | Viral myocarditis | 11/522 | 9.98E−04 | 11/585 | 2.50E−03 | — | >0.05 |
| hsa05418 | Fluid shear stress and | 18/522 | 2.75E−03 | 22/585 | 2.78E−04 | — | >0.05 |
| | atherosclerosis | | | | | | |

TABLE 7
Performance of the three predictive models with and without
APOE genotype in predicting other neurodegenerative diseases
when compared to Alzheimer's disease participants.

| | | | | Positive | Negative | |
|---|---|---|---|---|---|---|
| | | | | Predictive | Predictive | |
| Model | Status | Accuracy | Specificity | Value | Value | AUC |
| 40 | Parkinson's | 0.801 | 0.910 | 0.915 | 0.678 | 0.848 |
| transcripts | Disease | | | | | (0.799, 0.897) |
| model | Lewy Body | 0.602 | 0.910 | 0.455 | 0.836 | 0.554 |
| | Dementia | | | | | (0.401, 0.707) |
| | Frontotemporal | 0.799 | 0.910 | 0.647 | 0.924 | 0.864 |
| | Dementia | | | | | (0.763, 0.964) |
| 90 | Parkinson's | 0.738 | 0.881 | 0.875 | 0.608 | 0.768 |
| transcripts | Disease | | | | | (0.707, 0.828) |
| model | Lewy Body | 0.617 | 0.881 | 0.429 | 0.843 | 0.645 |
| | Dementia | | | | | (0.514, 0.776) |
| | Frontotemporal | 0.659 | 0.881 | 0.467 | 0.868 | 0.751 |
| | Dementia | | | | | (0.638, 0.864) |
| 220 | Parkinson's | 0.753 | 0.910 | 0.903 | 0.753 | 0.802 |
| transcripts | Disease | | | | | (0.746, 0.858) |
| model | Lewy Body | 0.632 | 0.910 | 0.500 | 0.632 | 0.686 |
| | Dementia | | | | | (0.561, 0.811) |
| | Frontotemporal | 0.611 | 0.910 | 0.455 | 0.611 | 0.757 |
| | Dementia | | | | | (0.645, 0.868) |
| 40 | Parkinson's | 0.784 | 0.791 | 0.839 | 0.716 | 0.861 |
| transcripts | Disease | | | | | (0.813, 0.908) |
| model & | Lewy Body | 0.559 | 1.000 | 1.000 | 0.817 | 0.653 |
| APOE | Dementia | | | | | (0.516, 0.790) |
| genotype | Frontotemporal | 0.790 | 0.955 | 0.769 | 0.914 | 0.938 |
| | Dementia | | | | | (0.892, 0.985) |
| 90 | Parkinson's | 0.716 | 0.687 | 0.769 | 0.657 | 0.793 |
| transcripts | Disease | | | | | (0.733, 0.853) |
| model & | Lewy Body | 0.559 | 1.000 | 1.000 | 0.817 | 0.689 |
| APOE | Dementia | | | | | (0.572, 0.806) |
| genotype | Frontotemporal | 0.618 | 0.985 | 0.800 | 0.846 | 0.777 |
| | Dementia | | | | | (0.668, 0.886) |
| 220 | Parkinson's | 0.731 | 0.716 | 0.787 | 0.731 | 0.818 |
| transcripts | Disease | | | | | (0.762, 0.874) |
| model & | Lewy Body | 0.581 | 0.985 | 0.750 | 0.581 | 0.724 |
| APOE | Dementia | | | | | (0.610, 0.838) |
| genotype | Frontotemporal | 0.548 | 0.970 | 0.500 | 0.548 | 0.797 |
| | Dementia | | | | | (0.687, 0.906) |

AUC = Area under the ROC Curve

What is claimed is:

1. A method of determining a type of neurodegenerative disease in a subject, the method comprising:
providing a biological sample obtained from the subject;
measuring a level of at least one gene-associated cfRNA in the biological sample; and
determining the type of neurodegenerative disease in the subject based on the level of the at least one gene-associated cfRNA.

2. The method of claim 1, wherein the type of neurodegenerative disease is selected from Alzheimer's Disease, Parkinson's disease, Lewy body dementia, and Frontotemporal dementia.

3. The method of claim 2, wherein the Alzheimer's Disease is selected from is preclinical, early symptomatic, or clinical Alzheimer's Disease.

4. The method of claim 3, wherein the subject has an APOE genotype risk factor for AD.

5. The method of claim 1, wherein a level of amyloid beta in the subject is not measured.

6. The method of claim 1, wherein the determining is not based on a level of amyloid beta in the subject.

7. The method of claim 1, wherein the biological sample is blood and wherein the level of gene-associated cfRNA is a level of gene-associated plasma cfRNA.

8. A method for detecting Alzheimer's Disease in a subject, the method comprising:
providing a biological sample obtained from the subject;
measuring a level of at least one gene-associated cfRNA in the biological sample; and
detecting Alzheimer's Disease in the subject based on the level of the at least one gene-associated cfRNA, wherein a level of amyloid beta in the subject is not measured and wherein the detecting is not based on a level of amyloid beta in the subject.

9. The method of claim 8, further comprising determining whether the Alzheimer's Disease is preclinical, early symptomatic, or clinical Alzheimer's Disease.

10. The method of claim 8, wherein the biological sample is blood and wherein the level of gene-associated cfRNA is a level of gene-associated plasma cfRNA.

11. The method of claim 10, wherein the at least one gene-associated cfRNA comprises CYTH1, PRPF8, SND1, and SLC9A3R2.

12. The method of claim 10, wherein the at least one gene-associated cfRNA comprises SYNPO.

13. The method of claim 12, wherein the at least one gene-associated cfRNA further comprises FP671120.3, JCAD, and PRPF8.

14. A method of selecting a treatment for a subject having a neurodegenerative disease, the method comprising:
providing a biological sample obtained from the subject;
measuring a level of at least one gene-associated cfRNA in the biological sample; and
selecting a treatment for the subject based on the level of the at least one gene-associated cfRNA.

15. The method of claim 14, wherein the neurodegenerative disease is Alzheimer's Disease.

16. The method of claim 15, wherein the Alzheimer's Disease is selected from is preclinical, early symptomatic, or clinical Alzheimer's Disease.

17. The method of claim 14, wherein the biological sample is blood and wherein the level of gene-associated cfRNA is a level of gene-associated plasma cfRNA.

18. The method of claim 17, wherein the at least one gene-associated cfRNA comprises CYTH1, PRPF8, SND1, and SLC9A3R2.

19. The method of claim 17, wherein the at least one gene-associated cfRNA comprises SYNPO.

20. The method of claim 19, wherein the at least one gene-associated cfRNA further comprises FP671120.3, JCAD, and PRPF8.
